(19) United States
(12) Patent Application Publication
(10) Pub. No.: US 2024/0279232 A1
(43) Pub. Date: Aug. 22, 2024

(54) USE OF SUBSTITUTED 5-(4-METHYL-6-PHENYL-4H-BENZO[F]IMIDAZO[1,5-A][1,4] DIAZEPIN-3-YL)-1,2,4-OXADIAZOLES IN THE TREATMENT OF INFLAMMATORY CONDITIONS

(71) Applicant: UWM Research Foundation, Inc., Milwaukee, WI (US)

(72) Inventors: Alexander E. Arnold, Milwaukee, WI (US); Douglas C. Stafford, Stoughton, WI (US); James M. Cook, Milwaukee, WI (US); Amanda N. Nieman, Hartford, WI (US); Michael Ming-Jin Poe, Fitchberg, WI (US); Guanguan Li, Shenzhen, Guangdong Province (CN)

(21) Appl. No.: 18/567,191

(22) PCT Filed: Jun. 7, 2021
(86) PCT No.: PCT/US2021/036215

§ 371 Date: Dec. 5, 2023

(51) Int. Cl. C07D 487/04; A61K 31/5517; A61P 29/00

(52) U.S. Cl. CPC C07D 487/04; A61K 31/5517; A61P 29/00

(57) ABSTRACT

Substituted 5-(4-methyl-6-phenyl-4H-benzo[f]imidazo[1,5-a][1.4]diazepin-3-yl)-1.2.4-oxadiazole compounds are ligands for the k opioid receptor and inhibit inducible nitric oxide synthase. The compounds have utility to inhibit NO production and treat associated inflammatory conditions.

### STATEMENT REGARDING FEDERAL FUNDING

This invention was made with government support under Grant numbers R⁰¹ MH096463, ROINS076517, and R⁰¹HL118561 awarded by the National Institutes of Health (NIH) and under Grant number CHE-1625735 awarded by the National Science Foundation. The government has certain rights in the invention.

### TECHNICAL FIELD

The present disclosure relates to compounds, compositions, and methods for treating or preventing nitric oxide (NO)-induced or aggravated diseases or disorders (e.g., inflammatory diseases).

### BACKGROUND

Nitric oxide (NO) generated by inducible nitric oxide synthase (INOS) activity has been implicated in a variety of diseases and conditions, including psoriasis, uveitis, Type 1 diabetes, septic shock, pain, migraine, rheumatoid arthritis, osteoarthritis, inflammatory bowel disease, asthma, immune complex diseases, multiple sclerosis, ischemic brain edema, toxic shock syndrome, heart failure, ulcerative colitis, atherosclerosis, glomerulonephritis, Paget's disease and osteoporosis, inflammatory sequelae of viral infections, retinitis, oxidant induced lung injury, eczema, acute allograft rejection, and infection caused by invasive microorganisms which produce NO.

In experimental animals, hypotension induced by lipopolysaccharide or tumor necrosis factor alpha can be reversed by NOS inhibitors. Conditions which lead to cytokine-induced hypotension include septic shock, hemodialysis and interleukin therapy in cancer patients. An iNOS inhibitor has been shown to be effective in treating cytokine-induced hypotension, inflammatory bowel disease, cerebral ischemia, osteoarthritis, and asthma. In addition, nitric oxide (NO) localized in high amounts in inflamed tissues has been shown to induce pain locally and to enhance central as well as peripheral stimuli.

Hence, in situations where the overproduction of nitric oxide is deleterious, it would be advantageous to find a specific inhibitor of iNOS to reduce the production of NO.

### SUMMARY

In one aspect, the invention provides a method of treating an inflammatory disease or disorder in a subject comprising administering the subject, a therapeutically effective amount of a compound of formula (1), or a pharmaceutically acceptable salt thereof,

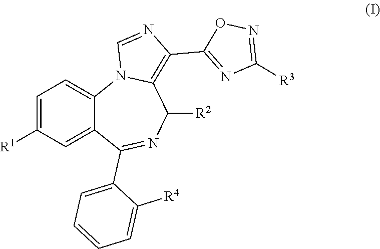

wherein:

R¹ is C_{3.4}cycloalkyl, halogen, ethynyl, —OC₁₋₂alkyl, or phenyl;

R² is methyl;

R³ is C₁₋₄alkyl or C₃₋₄cycloalkyl; and

R⁴ is hydrogen or halogen.

In another aspect, the invention provides a method of inhibiting inducible nitric oxide synthase (iNOS) in a subject comprising administering to the subject, an amount of a compound of formula (1), or a pharmaceutically acceptable salt thereof, that is effective to inhibit iNOS in the subject.

In another aspect, the invention provides a method of activating a x-opioid receptor (KOR) in a subject comprising administering to the subject, an amount of a compound of formula (I), or a pharmaceutically acceptable salt thereof, that is effective to activate KOR in the subject.

In another aspect, the invention provides compounds of formula (1), or pharmaceutically acceptable salts or compositions thereof.

### BRIEF DESCRIPTION OF THE DRAWINGS

FIG. 1A-1D show the verification of the expression of functional GABA_{A}R on immortalized microglia, cells were patch clamped at −80 mV and the current responses were acquired for 3 second applications of increasing concentrations of GABA.

FIG. 1A shows the current response with HMC3 human microglia cells.

FIG. 1B shows the 10 second average current sweeps of increasing concentrations of GABA with HMC3 cells.

FIG. 1C shows the current response with immortalized human and mouse microglia cells.

FIG. 1D shows the 10 second average current sweeps of increasing concentrations of GABA with immortalized human microglia cells.

FIG. 2A-2B show the expression of GABA_{A}R subunit mRNA isolated from immortalized human and mouse microglia. The mRNA (10 ng for brain extracts and 50 ng for microglia) was amplified with a QuantiFast SYBR green RT-qPCR kit (Quiagen) and cycle numbers for each subunit were normalized to GAPDH using the ΔCt method (n=3).

FIG. 2A shows the expression of GABA_{A}R subunit mRNA isolated from human brain extract.

FIG. 2B shows the expression of GABA_{A}R subunit mRNA isolated from human microglia.

FIG. 2C shows the expression of GABA_{A}R subunit mRNA isolated from mouse cerebellum extract.

FIG. 2D shows the expression of GABAAR subunit mRNA isolated from mouse microglia.

FIG. 3A-3B show the reduction of NO in the presence of MP-IV-010 and GABA_{A}R receptor antagonists. The data are presented as mean and SEM (n=8). *** indicate p<0.001, ns is no significance (ANOVA).

FIG. 3A shows mouse microglia activated with LPS/IFNγ and treated with indicated compound combinations. The NO levels of the media were quantified with a Griess assay after 14 h.

FIG. 3B shows the ATP levels of cells were quantified with CellTiter-Glo. The data are presented as mean and SEM (n=8), where *** indicate p<0.001, and ns indicates no significance (ANOVA).

FIG. 4A-4C show concentration-dependent receptor binding assays for MP-IV-010. The data are presented as mean and SEM (n=8). Non-linear regression was used to determine IC₅₀ values.

FIG. 4A shows the GABA_{A}R binding assay where the rat brain homogenate was incubated with ³H-flunitrazepam.

FIG. 4B shows the κ-opioid receptor binding assay where the cell lysate is from stably transfected HEK cells incubated with ³H-U69593.

FIG. 4C shows the σ2 receptor binding assay where the cell lysate is from stably transfected HEK cells incubated with ³H-ditolylguanidine.

FIG. 5A-5B show reduction of NO in the presence of MP-IV-010 and receptor antagonists. The data are presented as mean and SEM (n=8), where ** and *** indicate p<0.01 and p<0.001, and ns is no significance (ANOVA).

FIG. 5A shows the mouse microglia were activated with LPS/IFNγ and treated with indicated compound combinations. The NO levels of the media were quantified with a Griess assay after 14 h.

FIG. 5B shows ATP levels of cells were quantified with CellTiter-Glo.

FIG. 6A-6D show the measurement of iNOS mRNA, protein, and activity in activated and non-activated mouse microglia treated with vehicle or MP-IV-010 or GL-IV-03.

FIG. 6A shows that cellular protein extracts from activated microglia exhibited a significantly higher iNOS activity than extracts from non-activated microglia. MP-IV-010 (50 μM) treatment for 24 h completely inhibited the increased iNOS activity in activated microglia. The cellular proteins were isolated from mouse microglia treated with vehicle and mouse microglia activated with IFNγ/LPS and treated for 24 hours with vehicle or 50 μM MP-IV-010. The specific iNOS activity was determined with an Abcam INOS Activity Assay kit (n=4).

FIG. 6B shows that MP-IV-010 does not inhibit iNOS directly, cellular extracts from activated microglia were incubated with MP-IV-010 for 2 h followed by assay of iNOS activity. Here, iNOS activity did not differ from the vehicle treated cell extract. Cellular proteins were isolated from mouse microglia and mouse microglia activated for 24 hours with IFNγ/LPS. Cellular proteins from activated microglia were treated for 2 hours with 50 μM MP-IV-010 and iNOS activity was determined for all protein extracts with an Abcam INOS Activity Assay kit (n=3).

FIG. 6C shows that iNOS mRNA levels were reduced in activated microglia 15, 60, and 180 minutes after GL-IV-03 (10 μM) treatment. Mouse microglia were activated with IFNγ/LPS and treated with vehicle or 10 μM GL-IV-03. mRNA was isolated after 15 min, 1 h, 3 h and 6 h and iNOS was quantified using RT-qPCR. Cycle numbers for each sample were normalized to GAPDH using the ΔCt method (n=3). Mouse microglia were activated with IFNγ/LPS and treated with vehicle or 10 μM GL-IV-03 for 24 h. Cellular protein was extracted and analyzed with the mouse iNOS ELISA kit from Abcam (n=3). *, (p<0.05), ** (p<0.01) or *** (p<0.001) significance was calculate with ANOVA.

FIG. 6D shows the amount of iNOS protein isolated from mouse microglia after a 24 h treatment with GL-IV-03 was significantly lower in comparison to vehicle treated cells. FIG. 7A-7C shows the in vivo evaluation of MP-IV-010 and GL-IV-03.

FIG. 7A shows GL-IV-03 exhibited antinociceptive effects in both acute pain (phase 1) and inflammation mediated pain (phase 2) when administered orally at 10 mg/kg. Webster mice were treated orally four days in advance with indicated compounds and doses before the injection of 2% formalin in the right hind paw. Mice were evaluated during the first 5 min (phase 1) and after 20 minutes (phase 2) for 5 minutes in 5 seconds intervals Licking and biting the right hind paw during a 5 second interval was noted and combined as total time addressing the injected paw. Data is given as mean with SEM (n =8). ** indicate p<0.01 (unpaired t-test).

FIG. 7B shows that for both GL-IV-03 and MP IV-10 administered p.o. at 4-fold the effective dose in the formalin test did not cause any sensorimotor deficits as measured on the rotarod. Swiss Webster mice were tested on a rotarod at 15 rpm for 3 min at 10, 30, and 60 min following compound treatment at indicated dose and administration. The time of fall was recorded if it occurred prior to 3 min. Data are expressed as mean±SEM (n=10). *, (p<0.05), ** (p<0.01) or *** (p<0.001) significance compared to vehicle-treated mice (2 way ANOVA).

FIG. 7C shows that intraperitoneal injections of GL-IV-03 at 10 and 40 mg/kg did not influence the ability of mice to balance on a rotating rod for three minutes. Data are expressed as mean±SEM (n=10). *, (p<0.05), ** (p<0.01) or *** (p<0.001) significance compared to vehicle-treated mice (2-way ANOVA).

FIG. 8 shows the BRET recruitment assay which demonstrates that GL-1-30 is a full KOR agonist. HEK293 cells were transfected with Gα_{oA}-RLuc, Gβ3, Gγ8-GFP2 and human KOR and treated with increasing concentrations of GL-I-30 or full agonist salvinorin A.

FIG. 9 shows GL-I compounds.

FIG. 10 shows GL-II compounds.

FIG. 11 shows GL-III compounds.

FIG. 12 shows GL-III compounds.

FIG. 13 shows GL-IV compounds.

FIG. 14 shows various other compounds described herein.

FIG. 15A-15B shows the concentration response of compounds with significant reduction of NO production for mouse microglia. Means±SEM are presented for (n=12, *, **, and *** indicate p<0.05, p<0.01 and p<0.001 respectively).

FIG. 15A shows that cells were activated with LPS and IFNγ and treated with 1 μM GABA and 1 nM to 50 μM of compound for 24 hours. The NO in supernatant was quantified with a Griess assay.

FIG. 15B shows the ATP in remaining cells quantified by the Cell Titer Glo as a measure of viability.

FIG. 16A-16B shows the concentration response of compounds with significant reduction of NO production for mouse macrophages (RAW267.4).

FIG. 16A shows cells activated with LPS and IFNγ and treated with 1 μM GABA and 100 nM-50 μM of compound for 24 hours. NO in supernatant was quantified with a Griess assay.

FIG. 16B shows ATP in remaining cells was quantified by the Cell Titer Glo as a measure of viability. Means±SEM are presented for n=12. *, **, and *** indicate p<0.05, p<0.01 and p<0.001 respectively. (ANOVA)

FIG. 17A-17B shows the concentration response of compounds with significant reduction of NO production for mouse microglia.

FIG. 17A shows data for cells activated with LPS and IFNγ and treated with 1 μM GABA and 100 nM to 30 μM of compound for 24 hours. NO in supernatant was quantified with a Griess assay.

FIG. 17B shows ATP in remaining cells was quantified by the Cell Titer Glo as a measure of viability. Means±SEM are presented for n=12. *, **, and *** indicate p<0.05, p<0.01 and p<0.001 respectively. (ANOVA)

FIG. 18A-18B shows concentration response of compounds with significant reduction of NO production for mouse macrophages (RAW267.4).

FIG. 18A shows data for cells activated with LPS and IFNγ and treated with 1 μM GABA and 1 nM to 10 μM of imidazodiazepine compound for 24 hours. NO in supernatant was quantified with a Griess assay.

FIG. 18B shows ATP in remaining cells was quantified by the Cell Titer Glo as a measure of viability. Means±SEM are presented for n=12. *, **, and *** indicate p<0.05, p<0.01 and p<0.001 respectively. (ANOVA)

FIG. 19 shows concentration dependent viability of HepG2 (liver) and HEK293 (kidney) in the presence of compounds from plate 1. Cells were dispensed into 384 well plates and treated with different concentrations of compounds for 24 hours. The cell viability was determined with CellTiter-Glo (n=8 for each concentration)

FIG. 20 shows concentration dependent viability of HepG2 (liver) and HEK293 (kidney) in the presence of compounds from plate 2. Cells were dispensed into 384 well plates and treated with different concentrations of compounds for 24 hours. The cell viability was determined with CellTiter-Glo (n=8 for each concentration)

FIG. 21 shows concentration dependent viability of HepG2 (liver) and HEK293 (kidney) in the presence of compounds from plate 3. Cells were dispensed into 384 well plates and treated with different concentrations of compounds for 24 hours. The cell viability was determined with CellTiter-Glo. (n=8 for each concentration)

FIG. 22 shows concentration dependent viability of HepG2 (liver) and HEK293 (kidney) in the presence of compounds from plate 4. Cells were dispensed into 384 well plates and treated with different concentrations of compounds for 24 hours. The cell viability was determined with CellTiter-Glo. (n=8 for each concentration)

FIG. 23 shows concentration dependent viability of HepG2 (liver) and HEK293 (kidney) in the presence of compounds from plate 5. Cells were dispensed into 384 well plates and treated with different concentrations of compounds for 24 hours. The cell viability was determined with CellTiter-Glo. (n=8 for each concentration)

FIG. 24 shows concentration dependent viability of HepG2 (liver) and HEK293 (kidney) in the presence of compounds from plate 6. Cells were dispensed into 384 well plates and treated with different concentrations of compounds for 24 hours. The cell viability was determined with CellTiter-Glo. (n=8 for each concentration)

FIG. 25 shows concentration dependent viability of HepG2 (liver) and HEK293 (kidney) in the presence of compounds from plate 7. Cells were dispensed into 384 well plates and treated with different concentrations of compounds for 24 hours. The cell viability was determined with CellTiter-Glo. (n=8 for each concentration)

FIG. 26 shows concentration dependent viability of HepG2 (liver) and HEK293 (kidney) in the presence of compounds from plate 8. Cells were dispensed into 384 well plates and treated with different concentrations of compounds for 24 hours. The cell viability was determined with CellTiter-Glo. (n=8 for each concentration)

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed, inside cover, and specific functional groups are generally defined as described therein Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John

Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “halogen” or “halo,” as used herein, means Cl, Br, I, or F.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

### 2. Compounds

### A. Compounds of Formula (I)

Compounds useful in the invention are set forth in the following numbered embodiments. The first embodiment is denoted E1, another embodiment is denoted E2 and so forth.

E1. A compound of formula (I), or a pharmaceutically acceptable salt thereof,

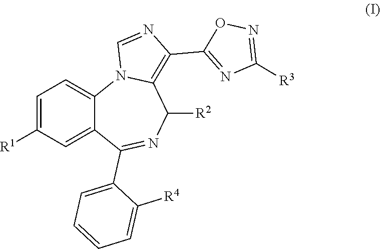

wherein:

R¹ is C₃₋₄cycloalkyl, halogen, ethynyl, —OC₁₋₂alkyl, or phenyl;

R² is methyl;

R³ is C₃₋₄alkyl or Czacycloalkyl; and

R⁴ is hydrogen or halogen.

E2. The compound of E1, or a pharmaceutically acceptable salt thereof, wherein R¹ is cyclopropyl.

E3. The compound of E1, or a pharmaceutically acceptable salt thereof, wherein R¹ is ethynyl.

E4. The compound of E1, or a pharmaceutically acceptable salt thereof, wherein R¹ is bromo.

E4.1. The compound of E1, or a pharmaceutically acceptable salt thereof, wherein R¹ is C₃₋₄cycloalkyl.

E4.2. The compound of E1, or a pharmaceutically acceptable salt thereof, wherein R¹ is halogen.

E4.3. The compound of E1, or a pharmaceutically acceptable salt thereof, wherein R¹ is —OC₁₋₂alkyl.

E4.4. The compound of E1, or a pharmaceutically acceptable salt thereof, wherein R¹ is phenyl.

E5. The compound of any of E1-E4.4, or a pharmaceutically acceptable salt thereof, wherein R³ is C₁₋₄alkyl.

E5.1. The compound of any of E1-E4.4, or a pharmaceutically acceptable salt thereof, wherein R³ is C₃₋₄cycloalkyl.

E6. The compound of any of E1-E5, or a pharmaceutically acceptable salt thereof, wherein R³ is methyl.

E7. The compound of any of E1-E5, or a pharmaceutically acceptable salt thereof, wherein R³ is ethyl.

E8. The compound of any of E1-E5, or a pharmaceutically acceptable salt thereof, wherein R³ is isopropyl.

E9. The compound of any of E1-E8, or a pharmaceutically acceptable salt thereof, R⁴ is fluoro.

E9.1. The compound of any of E1-E8, or a pharmaceutically acceptable salt thereof, wherein R⁴ is hydrogen.

E9.2. The compound of any of E1-E8, or a pharmaceutically acceptable salt thereof, wherein R⁴ is halogen.

E10. The compound of any of E1-E9.2, or a pharmaceutically acceptable salt thereof, wherein formula (I) is formula (Ia)

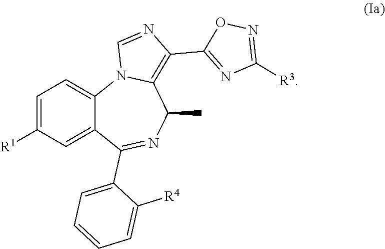

E11. The compound of any of E1-E9.2, or a pharmaceutically acceptable salt thereof, wherein formula (I) is formula (Ib)

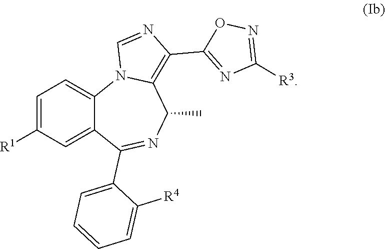

E12. The compound of E1, selected from the group consisting of

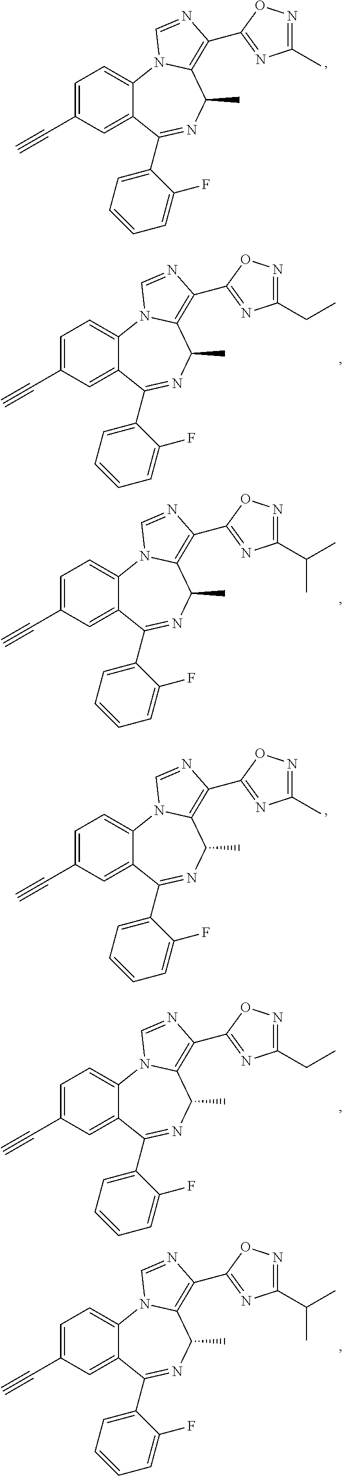

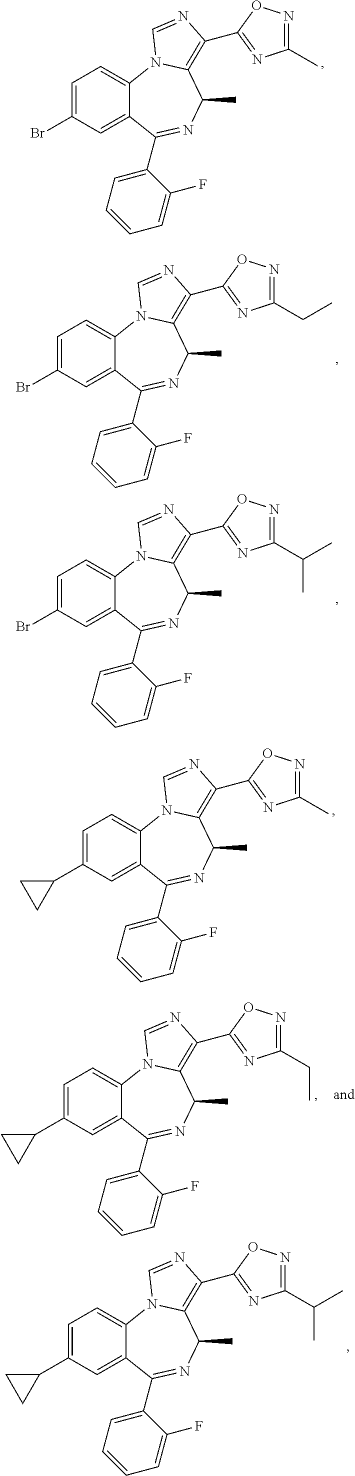

or a pharmaceutically acceptable salt thereof.

E13. The compound of E12 selected from the group consisting of

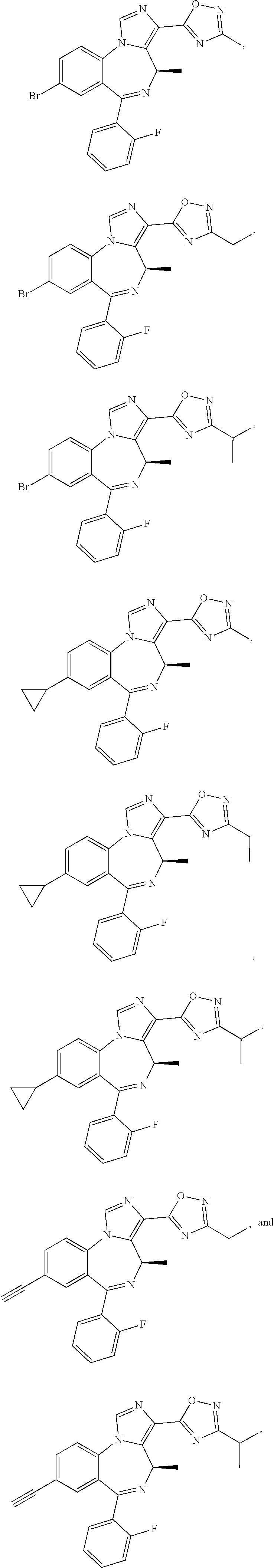

or a pharmaceutically acceptable salt thereof.

E14. A pharmaceutical composition comprising a compound of E13, or a pharmaceutically acceptable salt thereof, and a pharmaceutically acceptable carrier.

Compound names can be assigned by using Struct=Name naming algorithm as part of CHEMDRAW® ULTRA.

The compound may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

It should be understood that the compound may possess tautomeric forms, as well as geometric isomers, and that these also constitute an aspect of the invention.

In the compounds of formula (I) and any subformulas, any “hydrogen” or “H,” whether explicitly recited or implicit in the structure, encompasses hydrogen isotopes ¹H (protium) and ²H (deuterium).

### B. Pharmaceutical Salts

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, thrichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### 3. Pharmaceutical Compositions

The disclosed compounds may be incorporated into pharmaceutical compositions suitable for administration to a subject (such as a patient, which may be a human or non-human animal, such as a mammal).

The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention (e.g., a compound of formula (I)) are outweighed by the therapeutically beneficial effects A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

It will be appreciated that appropriate dosages of the compounds, and compositions comprising the compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician. In general, a suitable dose of the compound is in the range of about 100 μg to about 250 mg per kilogram body weight of the subject per day.

The composition may be administered once, on a continuous basis (e.g. by an intravenous drip), or on a periodic/intermittent basis, including about once per hour, about once per two hours, about once per four hours, about once per eight hours, about once per twelve hours, about once per day, about once per two days, about once per three days, about twice per week, about once per week, and about once per month. The composition may be administered until a desired reduction of symptoms is achieved.

The present compounds, compositions, and methods may be administered as part of a therapeutic regimen along with other treatments appropriate for the particular injury or disease being treated.

For example, a therapeutically effective amount of a compound of formula (I), may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their physiologically acceptable salts and solvates may be formulated for administration by, for example, solid dosing, eyedrop, in a topical oil-based formulation, injection, inhalation (either through the mouth or the nose), implants, or oral, buccal, parenteral, or rectal administration. Techniques and formulations may generally be found in “Remington's Pharmaceutical Sciences”, (Meade Publishing Co., Easton, Pa.). Therapeutic compositions must typically be sterile and stable under the conditions of manufacture and storage.

The route by which the disclosed compounds are administered, and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmelose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, PA) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and Mccutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of active [e.g., compound of formula (I)] and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmelose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound [e.g., a compound of formula (I)], and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT coatings (available from Rohm & Haas G.M.B.H. of Darmstadt, Germany), waxes and shellac

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

For parenteral administration, the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration, the agent can be in the form of an aqueous, lipid, oily or other kind of solution or suspension or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The pharmaceutical compositions of the present invention may also be administered by nasal aerosol or inhalation through the use of a nebulizer, a dry powder inhaler or a metered dose inhaler. Such compositions are prepared according to techniques well-known in the art of pharmaceutical formulation and may be prepared as solutions in saline, employing benzyl alcohol or other suitable preservatives, absorption promoters to enhance bioavailability, hydrofluorocarbons, and/or other conventional solubilizing or dispersing agents.

Aerosol propellants are required where the pharmaceutical composition is to be delivered as an aerosol under significant pressure. Such propellants include, e.g., acceptable fluorochlorohydrocarbons such as dichlorodifluoromethane, dichlorotetrafluoroethane, and trichloromonofluoromethane; nitrogen; or a volatile hydrocarbon such as butane, propane, isobutane or mixtures thereof.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I)), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

### 4. Methods of Treatment

### A. Diseases and Disorders

There exist three isoforms of NO synthases which fall into two classes and differ in their physiologic functions and molecular properties. The first class, known as constitutive NO synthases, comprises the endothelial NO synthase and the neuronal NO synthase. Both isoenzymes are expressed constitutively in various cell types, but are most prominent in endothelial cells of blood vessel walls (therefore called endothelial NO synthase, eNOS or NOS-III) and in neuronal cells (therefore called neuronal NO synthase, nNOS or NOS-I).

In contrast to the constitutive isoforms, the activation of inducible NO synthase (iNOS, NOS-II), the sole member of the second class, is performed by transcriptional activation of the iNOS promoter. For example, proinflammatory stimuli lead to transcription of the gene for inducible NO synthase, which is catalytically active without increases in the intracellular Ca²⁺ concentration. Due to the long half-life of the inducible NO synthase and the unregulated activity of the enzyme, high micromolar concentrations of NO are generated over longer time periods. These high NO concentrations alone or in cooperation with other reactive radicals are cytotoxic. Therefore, in situations of microbial infections, iNOS is involved in cell killing by macrophages and other immune cells during early nonspecific immune responses.

Among the three isoforms of NOS (neuronal, endothelial, and inducible or iNOS), iNOS is expressed in immune cells, such as macrophages, and it is the only form of NOS to be reported in microglia (Sierra et al., PLOS One 9, e106048). Microglial activation induces the expression of iNOS that in turn produces large cytotoxic quantities of NO.

A number of pro-inflammatory cytokines and endotoxin (bacterial lipopolysaccharide, LPS) also induce the expression of iNOS in a number of cells, including macrophages, vascular smooth muscle cells, epithelial cells, fibroblasts, glial cells, cardiac myocytes as well as vascular and non-vascular smooth muscle cells. Although monocytes/macrophages are the primary source of iNOS in inflammation, LPS and other cytokines induce a similar response in astrocytes and microglia.

There are a number of pathophysiological situations, which among others, are characterized by the high expression of inducible NO synthase and concomitant high NO. It has been shown that these high NO concentrations alone or in combination with other radical species lead to tissue and organ damage and are causally involved in these pathophysiologies. As inflammation is characterized by the expression of proinflammatory enzymes, including inducible NO synthase, selective inhibitors of inducible NO synthase can be used as therapeutics for diseases involving acute and chronic inflammatory processes. Other pathophysiologies with high NO production from inducible NO synthase are several forms of shock (e.g. septic, hemorrhagic and cytokine-induced shock).

There is now substantial evidence that iNOS plays an important role in the pathogenesis of a variety of diseases. In addition, excess NO production is involved in several disorders, including disorders that involve systemic hypotension such as septic and toxic shock and therapy with certain cytokines. Circulatory shock of various etiologies is associated with profound changes in the body's NO homeostasis. In animal models of endotoxic shock, endotoxin produces an acute release of NO from the constitutive isoform of nitric oxide synthase in the early phase, which is followed by induction of INOS. NO derived from macrophages, microglia and astrocytes has been implicated in the damage of myelin producing oligodendrocytes in demyelinating disorders like multiple sclerosis and neuronal death during neuronal degenerating conditions including brain trauma.

Astrocytes, the predominant glial component of brain have also been shown to induce iNOS in response to bacterial lipopolysaccharide (LPS) and a series of proinflammatory cytokines including interleukin-1β (IL-1β), tumor necrosis factor-α (TNF-α), and interferon-y (IFN-γ).

Cytokines associated with extracellular signaling are involved in the normal process of host defense against infections and injury, in mechanisms of autoimmunity and in the pathogenesis of chronic inflammatory diseases. It is believed that nitric oxide (NO), synthesized by nitric oxide synthetase (NOS) mediates deleterious effects of the cytokines. For example, NO as a result of stimuli by cytokines (e.g., TNF-α, IL-1 and interleukin-6 (IL-6)) is implicated in autoimmune diseases such as multiple sclerosis, rheumatoid arthritis, and osteoarthritis. The NO produced by iNOS is associated with bactericidal properties of macrophages. Recently, an increasing number of cells (including muscle cells, macrophages, keratinocytes, hepatocytes and brain cells) have been shown to induce iNOS in response to a series of proinflammatory cytokines including IL-1, TNF-α, interferon-γ (IFN-γ), and bacterial lipopolysaccharides (LPS).

NO generated by iNOS has been implicated in the pathogenesis of inflammatory diseases. Experiments have demonstrated that animal hypotension induced by LPS or TNF-α can be reversed by NOS inhibitors and reinitiated by L-arginine. Conditions which lead to cytokine induced hypotension include septic shock, hemodialysis, and IL-2 therapy in cancer patients. Studies in animal models have suggested a role for NO in the pathogenesis of inflammation and pain and NOS inhibitors have been shown to have beneficial effects on some aspects of the inflammation and tissue changes seen in models of inflammatory bowel diseases, cerebral ischemia and arthritis.

NO generated by iNOS activity has been implicated in a variety of diseases and conditions, including dermatitis, psoriasis, uveitis, Type 1 diabetes, septic shock, pain, migraine, rheumatoid arthritis, osteoarthritis, inflammatory bowel disease, asthma, immune complex diseases, multiple sclerosis, ischemic brain edema, toxic shock syndrome, heart failure, ulcerative colitis, atherosclerosis, glomerulonephritis, Paget's disease and osteoporosis, inflammatory sequelae of viral infections, retinitis, oxidant induced lung injury, eczema, acute allograft rejection, and infection caused by invasive microorganisms which produce NO.

It has been shown in in-vivo animal models of septic shock that reduction of circulating plasma NO levels by NO scavenger or inhibition of inducible NO synthase restores systemic blood pressure, reduces organ damage and increases survival (deAngelo, Exp. Opin. Pharmacother. 19-29, 1999; Redl et al., Shock 8, Suppl. 51, 1997; Strand et al., Crit. Care Med. 26, 1490-1499, 1998). It has also been shown that increased NO production during septic shock contributes to cardiac depression and myocardial dysfunction (Sun et al. J. Mal., Cell Cardiol. 30, 989-997, 1998). There are also reports showing reduced infarct size after occlusion of the left anterior coronary artery in the presence of NO synthase inhibitors (Wang et al. Am. J., Hypertens. 12, 174-182, 1999). Considerable inducible NO synthase activity is found in human cardiomyopathy and myocarditis, supporting the hypothesis that NO accounts at least in part for the dilatation and impaired contractility in these pathophysiologies (de Seider et al., Br. Heart. J. 4, 426-430, 1995).

In animal models of acute or chronic inflammation, blockade of inducible NO synthase by isoform-selective or nonselective inhibitors or genetic knock out improves therapeutic outcome. It is reported that experimental arthritis (Connor et al., Eur. J. Pharmacol. 273, 15-24, 1995) and osteoarthritis (Pelletier et al., Arthritis & Rheum. 41, 1275-1286, 1998), experimental inflammations of the gastrointestinal tract (Zingarelli et al., Gut 45, 199-209, 1999), experimental glomerulonephritis (Narita et al., Lab. Invest. 72, 17-24, 1995), experimental diabetes (Corbett et al., PNAS 90, 8992-8995, 1993), and lipopolysaccharide-induced experimental lung injury is reduced by inhibition of inducible NO synthase or in iNOS knock out mice (Kristof et al., Am. J. Crit. Care. Med. 158, 1883-1889, 1998). A pathophysiological role of inducible NO synthase derived NO or O₂— is also discussed in chronic inflammatory diseases, such as asthma, bronchitis and chronic obstructive pulmonary disease (COPD).

In models of neurodegenerative diseases of the central nervous system such as MPTP-induced parkinsonism (MPTP =1-methyl-4-phenyl-1,2,3,6-tetrahydropyridine), amyloid peptide induced Alzheimer's disease (Ishii et al., FASEB J. 14, 1485-1489, 2000), malonate induced Huntington's disease (Connop et al., Neuropharmacol. 35, 459-465, 1996), experimental meningitis (Korytko & Boje, Neuropharmacol. 35, 231-237, 1996) and experimental encephalitis (Parkinson et al., J. Mal. Med. 75, 174-186, 1997) a causal participation of NO and inducible NO synthase has been shown.

Other studies implicated nitric oxide as a potential mediator of microglia dependent primary demyelination, a hallmark of multiple sclerosis (Parkinson et al., J. Mal. Med. 75, 174-186, 1997).

An inflammatory reaction with concomitant expression of inducible NO synthase also takes place during cerebral ischemia and reperfusion (Iadecola et al., Stroke 27, 1373-1380, 1996). Resulting NO together with superoxide from infiltrating neutrophils is thought to be responsible for cellular and organ damage.

On account of their inducible NO synthase inhibiting properties, the compounds according to the invention can be employed in human and veterinary medicine, where an excess of NO due to increases in the activity of inducible NO synthase is involved. In particular, they can be used without limitation for the treatment and prophylaxis of the following inflammatory diseases, disorders, or conditions:

Acute inflammatory diseases: Septic shock, sepsis, systemic inflammatory response syndrome (SIRS), hemorrhagic shock, shock states induced by cytokine therapy (interleukin-2, tumor necrosis factor, immune checkpoint inhibition), organ transplantation and transplant rejection, head trauma, acute lung injury, acute respiratory distress syndrome (ARDS), inflammatory skin conditions such as sunburn, inflammatory eye conditions such as uveitis, glaucoma and conjunctivitis.

Chronic inflammatory diseases, in particular chronic inflammatory diseases of peripheral organs and the CNS: gastrointestinal inflammatory diseases such as Crohn's disease, inflammatory bowel disease, ulcerative colitis, lung inflammatory diseases such as asthma, chronic bronchitis, emphysema and COPD, inflammatory diseases of the upper respiratory tract such as allergic rhinitis and allergic sinusitis, inflammatory eye conditions such as allergic conjunctivitis, arthritic disorders such as rheumatoid arthritis, osteoarthritis and gouty arthritis, heart disorders such as cardiomyopathy and myocarditis, atherosclerosis, neurogenic inflammation, skin diseases such as psoriasis, pruritus, dermatitis and eczema, diabetes, glomerulonephritis; Parkinson's disease, amyotrophic lateral sclerosis (ALS), multiple sclerosis; necrotizing vasculitides such as polyarteritis nodosa, serum sickness, Wegener's granulomatosis, Kawasaki's syndrome; headaches such as migraine, chronic tension headaches, cluster, and vascular headaches; urological disorders such as overactive bladder and cystitis; and myocardial and cerebral ischemia/reperfusion injury.

A respiratory disease or condition including: asthmatic conditions including allergen-induced asthma, exercise-induced asthma, pollution-induced asthma, cold-induced asthma, and viral-induced-asthma; chronic obstructive pulmonary diseases including chronic bronchitis with normal airflow, chronic bronchitis with airway obstruction (chronic obstructive bronchitis), emphysema, asthmatic bronchitis, and bullous disease; and other pulmonary diseases involving inflammation including bronchioectasis cystic fibrosis, pigeon fancier's disease, farmer's lung, acute respiratory distress syndrome, pneumonia, aspiration or inhalation injury, fat embolism in the lung, acidosis inflammation of the lung, acute pulmonary edema, acute mountain sickness, acute pulmonary hypertension, persistent pulmonary hypertension of the newborn, perinatal aspiration syndrome, hyaline membrane disease, acute pulmonary thromboembolism, heparin-protamine reactions, sepsis, status asthmaticus and hypoxia.

Inflammatory pain, pain syndromes such as ocular pain, pain such as surgical analgesia, or as an antipyretic for the treatment of fever, post-surgical pain for various surgical procedures including post-cardiac surgery, dental pain/dental extraction, pain resulting from cancer, muscular pain, mastalgia, pain resulting from dermal injuries, lower back pain, headaches of various etiologies, including migraine, and the like, pain-related disorders such as tactile allodynia and hyperalgesia.

Insulin resistance and other metabolic disorders such as atherosclerosis that are typically associated with an exaggerated inflammatory signaling.

Arthritis, including but not limited to rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis.

Osteoporosis and other Related Bone Disorders.

Gastrointestinal conditions such as reflux esophagitis, diarrhea, inflammatory bowel disease, Crohn's disease, gastritis, irritable bowel syndrome, ulcerative colitis, celiac disease, gastritis, pancreatitis, proctitis, hepatitis, diverticulitis, and tropical sprue.

Pulmonary inflammation, such as that associated with viral infections and cystic fibrosis.

Dermatologic disease or condition, such as dermatitis, atopic dermatitis, rash, pruritis, ankylosing spondylitis, eczema, acne, dandruff, cellulitis, psoriasis, rosacea, hives, shingles, lupus erythematosus, lichen planus, dermatitides, vasculitis, bullous diseases, and vitiligo.

Tissue damage in such diseases as vascular diseases, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephritis, nephrotic syndrome, Behcet's syndrome, polymyositis, gingivitis, periodontis, hypersensitivity, swelling occurring after injury, ischemias including myocardial ischemia, cardiovascular ischemia, and ischemia secondary to cardiac arrest, and the like.

Central nervous system disorders such as central nervous system damage resulting from stroke, ischemias including cerebral ischemia (both focal ischemia, thrombotic stroke and global ischemia (for example, secondary to cardiac arrest), and trauma.

Systemic hypotension associated with septic and/or toxic hemorrhagic shock induced by a wide variety of agents; therapy with cytokines such as TNF, IL-1 and IL-2.

Cancers that express nitric oxide synthase, such as colorectal cancer, and cancer of the breast, lung, prostate, bladder, cervix and skin, neoplasias including but not limited to brain cancer, bone cancer, a leukemia, a lymphoma, epithelial cell-derived neoplasia (epithelial carcinoma) such as basal cell carcinoma, adenocarcinoma, gastrointestinal cancer such as lip cancer, mouth cancer, esophogeal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that effect epithelial cells throughout the body.

Ophthalmic diseases, such as glaucoma, retinal ganglion degeneration, ocular ischemia, retinitis, retinopathies, uveitis, ocular photophobia, and of inflammation and pain associated with acute injury to the eye tissue, glaucomatous retinopathy and/or diabetic retinopathy, post-operative inflammation or pain as from ophthalmic surgery such as cataract surgery and refractive surgery.

The invention further relates to a method of treating or preventing one of the above-mentioned diseases in a mammal, including a human, in need thereof comprising administering a therapeutically effective amount of at least one of the compounds according to the invention.

Especially, the invention relates to a method of treating or preventing a disease which is alleviated by inhibition of inducible nitric oxide synthase in a mammal, including a human, in need thereof comprising administering a therapeutically effective amount of at least one of the compounds according to the invention.

In particular, the invention relates to a method of treating or preventing an acute or chronic inflammatory disease, in particular sepsis, septic shock, systemic inflammatory response syndrome, hemorrhagic shock, shock states induced by cytokine therapy, asthma, chronic obstructive pulmonary disease, allergic rhinitis, cardiomyopathy or myocarditis, in a mammal, including a human, in need thereof comprising administering a therapeutically effective amount of at least one of the compounds according to the invention.

Compounds of the invention may bind to and activate the κ-opioid receptor (KOR). Without being bound by theory, compounds of the invention may inhibit NO production by activation of the KOR, resulting in reduced expression of iNOS. Opioid receptor agonist biphalin, a dimeric enkephalin, reduced iNOS expression in addition proinflammatory factors such as IL-1B, IL-18, COX-2, and NLRP3 in LPS-treated primary microglial cells (Popiolek-Barczyk et al. Neural Plast. 2017, 3829472). The transcriptional effects were mediated by diminished levels of p-NF-κB, p-IκB, p-p38MAPK, and TRIF that were reversible by opioid receptor antagonist naloxone. It has been observed that the selective KOR agonist salvinorin A (SA) inhibits iNOS and COX-2 expression in lipopolysaccharide (LPS) activated alveolar macrophages (AMs), macrophages that produce and release a variety of pro- and anti-inflammatory cytokines to modulate the immune response. The SA treatment reduced NO production within 2 hours of LPS treatment (Zeng et al., Transl Perioper Pain Med, 2020, 7(3), 225-233). Consistent with this finding, it was observed that KOR agonists down-regulated nitrite levels, which are the stable metabolites of NO, indicating reduced NO production. Thus, the use of KOR agonists that preferentially target the iNOS/NO pathway may treat diseases or disorders induced or aggravated by NO.

One aspect of the invention provides a method of treating an inflammatory disease or disorder in a subject comprising administering to the subject, a therapeutically effective amount of a compound of any of E1-E13, or a pharmaceutically acceptable salt thereof.

Another aspect of the invention provides a method of inhibiting inducible nitric oxide synthase (iNOS) in a subject comprising administering to the subject, an amount of a compound of any of E1-E13, or a pharmaceutically acceptable salt thereof, that is effective to inhibit iNOS in the subject.

Another aspect of the invention provides a method of activating a κ-opioid receptor (KOR) in a subject comprising administering to the subject, an amount of a compound of any of E1-E13, or a pharmaceutically acceptable salt thereof, that is effective to activate KOR in the subject.

Another aspect of the invention provides use of a compound of any of E1-E13, or a pharmaceutically acceptable salt thereof, in the manufacture of a medicament for treating an inflammatory disease or disorder.

Another aspect of the invention provides use of a compound of any of E1-E13, or a pharmaceutically acceptable salt thereof, in the manufacture of a medicament for inhibiting inducible nitric oxide synthase (iNOS).

Another aspect of the invention provides use of a compound of any of E1-E13, or a pharmaceutically acceptable salt thereof, in the manufacture of a medicament for activating a κ-opioid receptor (KOR).

### B. Combination Therapies

Additional therapeutic agent(s) may be administered simultaneously or sequentially with the disclosed compounds and compositions. Sequential administration includes administration before or after the disclosed compounds and compositions. In some embodiments, the additional therapeutic agent or agents may be administered in the same composition as the disclosed compounds. In other embodiments, there may be an interval of time between administration of the additional therapeutic agent and the disclosed compounds. In some embodiments, administration of an additional therapeutic agent with a disclosed compound may allow lower doses of the other therapeutic agents and/or administration at less frequent intervals. When used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of any one of formulas (I)-(III).

Specific, non-limiting examples of possible combination therapies include use of the compounds of the invention with: a) corticosteroids including betamethasone dipropionate (augmented and nonaugemented), betamethasone valerate, clobetasol propionate, diflorasone diacetate, halobetasol propionate, amcinonide, dexosimethasone, fluocinolone acetononide, fluocinonide, halocinonide, clocortalone pivalate, dexosimetasone, and flurandrenalide; b) non-steroidal anti-inflammatory drugs including diclofenac, ketoprofen, and piroxicam; c) muscle relaxants and combinations thereof with other agents, including cyclobenzaprine, baclofen, cyclobenzaprine/lidocaine, baclofen/cyclobenzaprine, and cyclobenzaprine/lidocaine/ketoprofen; d) anaesthetics and combinations thereof with other agents, including lidocaine, lidocaine/deoxy-D-glucose (an antiviral), prilocaine, and EMLA Cream [Eutectic Mixture of Local Anesthetics (lidocaine 2.5% and prilocaine 2.5%; an emulsion in which the oil phase is a eutectic mixture of lidocaine and prilocaine in a ratio of 1:1 by weight. This eutectic mixture has a melting point below room temperature and therefore both local anesthetics exist as a liquid oil rather than as crystals)]; e) expectorants and combinations thereof with other agents, including guaifenesin and guaifenesin/ketoprofen/cyclobenzaprine; f) antidepressants including tricyclic antidepressants (e.g., amitryptiline, doxepin, desipramine, imipramine, amoxapine, clomipramine, nortriptyline, and protriptyline), selective serotonin/norepinephrine reuptake inhibitors including (e.g, duloxetine and mirtazepine), and selective norepinephrine reuptake inhibitors (e.g., nisoxetine, maprotiline, and reboxetine), selective serotonin reuptake inhibitors (e.g., fluoxetine and fluvoxamine); g) anticonvulsants and combinations thereof, including gabapentin, carbamazepine, felbamate, lamotrigine, topiramate, tiagabine, oxcarbazepine, carbamezipine, zonisamide, mexiletine, gabapentin/clonidine, gabapentin/carbamazepine, and carbamazepine/cyclobenzaprine; h) antihypertensives including clonidine; i) opioids including loperamide, tramadol, morphine, fentanyl, oxycodone, levorphanol, and butorphanol; j) topical counter-irritants including menthol, oil of wintergreen, camphor, eucalyptus oil and turpentine oil; k) topical cannabinoids including selective and non-selective CB1/CB2 ligands; and other agents, such as capsaicin.

The present compounds may also be used in co-therapies, partially or completely, in place of other conventional anti-inflammatory therapies, such as together with steroids, NSAIDs, COX-2 selective inhibitors, 5-lipoxygenase inhibitors, LTB₄ antagonists and LTA₄ hydrolase inhibitors. The compounds of the subject invention may also be used to prevent tissue damage when therapeutically combined with antibacterial or antiviral agents.

The disclosed compounds may be included in kits comprising the compound [e.g., one or more compounds of formula (I)], a systemic or topical composition described above, or both; and information, instructions, or both that use of the kit will provide treatment for medical conditions in mammals (particularly humans). The information and instructions may be in the form of words, pictures, or both, and the like. In addition or in the alternative, the kit may include the medicament, a composition, or both; and information, instructions, or both, regarding methods of application of medicament, or of composition, preferably with the benefit of treating or preventing medical conditions in mammals (e.g., humans).

The compounds and processes of the invention will be better understood by reference to the following examples, which are intended as an illustration of and not a limitation upon the scope of the invention.

### EXAMPLES

### A. Synthesis of Compounds

Compounds of formula (I) may be prepared by synthetic processes or by metabolic processes. Preparation of the compounds by metabolic processes includes those occurring in the human or animal body (in vivo) or processes occurring in vitro.

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Optimum reaction conditions and reaction times for each individual step can vary depending on the particular reactants employed and substituents present in the reactants used. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### Abbreviations:

t-BuOK potassium tert-butoxide
CIPO(OEt)₂ diethyl chlorophosphate
CNCH₂CO₂Et ethyl isocyanoacetate
eq equivalent(s)
EtOAc ethyl acetate
Et₃N triethylamine
h hour(s)
IPA isopropyl alcohol
min minute(s)
Pd(OAc)₂ palladium acetate
rt room temperature
TFA trifluoroacetic acid
THF tetrahydrofuran
TLC thin layer chromatography
   Scheme 1. General Synthesis of Substituted 3-Methyl-5-phenyl-1,3-dihydro-2H-benzo[e][1,4]diazepin-2-ones

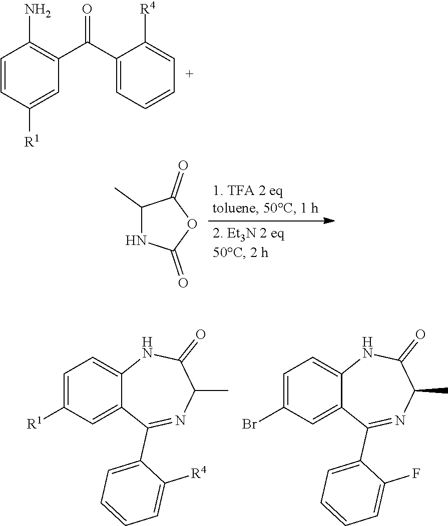

Preparation of (R)-7-bromo-5-(2-fluorophenyl)-3-methyl-1,3-dihydro-2H-benzo[e][1,4]diazepin-2-one. A mixture of 2-amino-5-bromo-2′-fluorobenzophenone (140.7 g, 478.4 mmol) and trifluoroacetic acid (73.3 mL, 956.7 mmol) in anhydrous toluene (2200 mL) was stirred at room temperature for 30 min to form a solution. N-carboxy-D-alanine anhydride (66.0 g, 574.0 mmol) was added and the reaction mixture was heated for 1 h at 50° C. After confirmation of >95% conversion of 2-amino-5-bromo-2′-fluorobenzophenone (TLC, 50% ethyl acetate in hexanes), triethylamine (133.3 mL, 956.7 mmol) was added dropwise to the reaction mixture over 30 min, while maintaining the temperature at 50° C. After 2 h at 50° C., the intermediate TFA salt was consumed (TLC, 50% ethyl acetate in hexanes). Upon cooling the reaction mixture to room temperature, solvents were removed under reduced pressure and the residue was dissolved in ethyl acetate (1500 mL) and water (1500 mL). The resulting biphasic mixture was separated and the organic layer was washed with 5% aqueous sodium bicarbonate solution (1500 mL) followed by 10% aqueous sodium chloride solution (1500 mL). The organic layer was dried over Na2SO4 and evaporated under reduced pressure. 10% Ethyl acetate/heptane was added to the residue and evaporated (25 mL×2) and the residue was treated with 10% ethyl acetate in heptane (1700 mL) at 60° C. for 30 min to dissolve unreacted starting material. The reaction mixture was cooled to room temperature and stirred for an additional 2 h before the product was collected by filtration and washed with 10% ethyl acetate in heptane (50 mL×2) followed by heptane (50 mL×2). The solid was dried under vacuum at 40° C. to afford the title compound as an off-white solid (127.0 g, 77.0%): 1H NMR (500 MHz, CDCl3) δ 9.69 (s, 1H), 7.63-7.59 (m, 1H), 7.59 (dd, J=8.5, 2.3 Hz, 1H), 7.47 (dddd, J=8.2, 7.0, 5.0, 1.8 Hz, 1H), 7.36 (d, J=2.2 Hz, 1H), 7.26 (td, J=7.5, 1.1 Hz, 1H), 7.12 (d, J=8.6 Hz, 1H), 7.08 (ddd, J=10.2, 8.3, 1.1 Hz, 1H), 3.79 (q, J=6.5 Hz, 1H), 1.78 (d, J=6.5 Hz, 3H); 13C NMR (126 MHZ, CDCl3) δ 172.39 (s), 164.53 (s), 160.45 (d, 1JCF=251.9 Hz), 136.47 (s), 134.74 (s), 132.19 (d, 3JCF=8.3 Hz), 132.03 (d, JCF=1.5 Hz, NOE coupling), 131.56 (d, 3JCF=2.2 Hz), 130.15 (s), 127.13 (d, 2JCF=12.4 Hz), 124.46 (d, 4JCF=3.6 Hz), 122.98 (s), 116.54 (s), 116.29 (d, 2JCF=21.5 Hz), 58.85 (s), 16.93 (s); 19F NMR (471 MHz, CDCl3) δ−112.53; HRMS (ESI/IT-TOF): m/z [M+H]+ calcd for C16H13BrFN2O: 347.0190; found: 347.0181; HPLC Purity: 99.2%; Optical Purity: 98.8% ee.

Scheme 2. General Synthesis of Substituted Ethyl 4-methyl-6-phenyl-4H-benzo[f]imidazo[1,5-a][1,4]diazepine-3-carboxylates

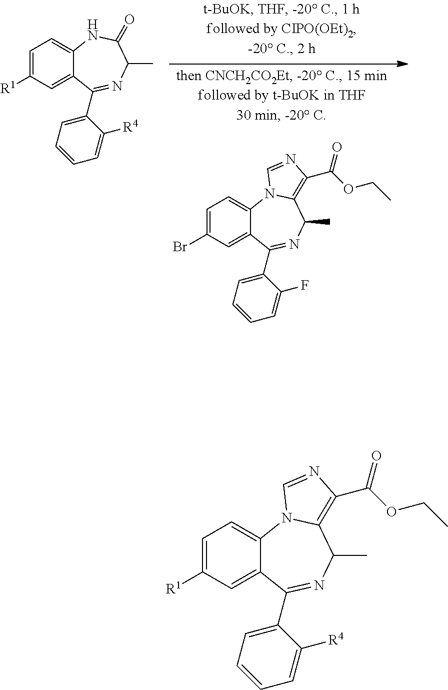

Ethyl (R)-8-bromo-6-(2-fluorophenyl)-4-methyl-4H-benzo[f]imidazo[1,5-a] [1,4]diazepine-3-carboxylate. (R)-7-Bromo-5-(2-fluorophenyl)-3-methyl-1,3-dihydro-2H-benzo[e][1,4]diazepin-2-one (125.0 g, 360.0 mmol) in anhydrous tetrahydrofuran (2000 mL) was cooled to −20° C. using a dry ice/IPA bath. A solution of t-BuOK (52.5 g, 468.0 mmol) in tetrahydrofuran (300 mL) was added dropwise to the reaction mixture over 30 min, while maintaining a temperature of −20° C. Upon completion of the addition, the reaction mixture was allowed to stir for an additional 60 min at −20° C. Diethyl chlorophosphate (72.8 mL, 504.1 mmol) was then added dropwise to the reaction mixture over 15 min at −20° C. After 2 h at −20° C., the starting material was consumed (TLC, 100% ethyl acetate). Ethyl isocyanoacetate (51.2 mL, 468.0 mmol) was added dropwise over 15 min while maintaining −20° C., followed by the dropwise addition of a solution of t-BuOK (52.5 g, 468.0 mmol) in tetrahydrofuran (300 mL) over 30 min at −20° C. Upon completion of the addition, the reaction mixture was allowed to warm to room temperature and stir for an additional 1 h, at which point the intermediate was fully consumed (TLC, 100% ethyl acetate). The reaction mixture was diluted with 5% aqueous sodium bicarbonate (2000 mL) and ethyl acetate (2000 mL). The resulting emulsion was filtered and the layers separated after 30 min. The aqueous layer was extracted with ethyl acetate (2000 mL) and the combined organic layers were washed with 10% aqueous sodium bicarbonate solution (2000 mL) and 20% aqueous sodium chloride solution (2000 mL). The organic layer was dried over Na₂SO₄ and evaporated under reduced pressure. t-Butyl methyl ether was added to the residue and evaporated (200 mL×2) and the residue was treated with t-butyl methyl ether (1000 mL) at 55° C. for 30 min. The mixture was stirred for 12 h at room temperature followed by filtration and washing with t-butyl methyl ether (100 mL×4). The solid was dried under vacuum at 40° C. to yield the title compound as a white powder (96.6 g, 60.7%): 1H NMR (500 MHz, CDCl3) δ 7.92 (s, 1H), 7.73 (dd, J=8.5, 2.2 Hz, 1H), 7.60 (dt, J=7.3, 3.9 Hz, 1H), 7.48 (d, J=8.6 Hz, 1H), 7.50-7.42 (m, 1H), 7.42 (d, J=2.2 Hz, 1H), 7.26 (td, J=7.5, 1.1 Hz, 1H), 7.10-7.02 (m, 1H), 6.71 (q, J=7.3 Hz, 1H), 4.54-4.28 (m, 2H), 1.42 (t, J=7.1 Hz, 3H), 1.29 (d, J=7.4 Hz, 3H); 13C NMR (126 MHz, CDCl3) δ 162.93 (s), 162.67 (s), 160.10 (d, 1JCF=250.7 Hz), 141.59 (s), 134.85 (s), 134.75 (s), 133.68 (s), 133.05 (s), 132.12 (d, 3JCF=8.2 Hz), 131.17 (s), 129.59 (s), 128.42 (d, 2JCF=12.3 Hz), 124.57 (d, 4JCF=3.3 Hz), 123.65 (s), 120.96 (s), 116.25 (d, 2JCF=21.4 Hz), 60.82 (s), 50.12 (s), 14.87 (s), 14.43 (s); 19F NMR (471 MHZ, CDCl3) δ-112.36; HRMS (ESI/IT-TOF): m/z [M+H]+ calcd for C21H18BrFN3O2: 442.0561; found: 442.0563; HPLC Purity: 97.5%; Optical Purity: 99.0% ee.

Scheme 3. General Synthesis of Substituted 5-(4-Methyl-6-phenyl-4H-benzo[f]imidazo[1,5-a][1,4]diazepin-3-yl)-1,2,4-oxadiazoles

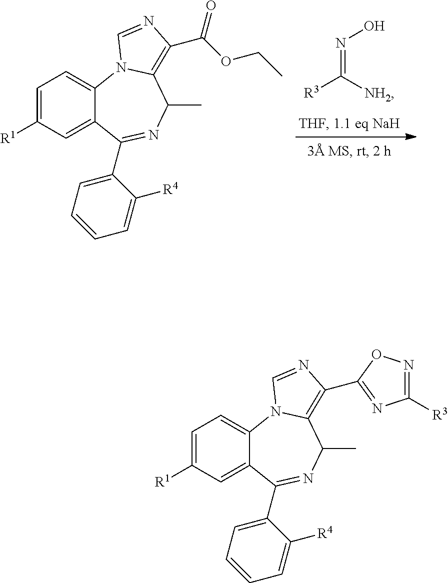

General Procedure for the Preparation of Oxadiazoles. The ethyl esters (0.52 mmol) are dissolved in dry THF (20 mL) at rt under argon. In a separate flask which contained 3 Å molecular sieves, the corresponding amide oxime R³C(═NOH)NH₂ (2.08 mmol) is dissolved in dry THF (30 mL) under argon and treated with sodium hydride (60% dispersion in mineral oil, 0.57 mmol). The resulting mixture is stirred for 15 min, at which point the solution containing the ethyl ester is added. The resulting reaction mixture is stirred at rt for 2 h until the starting material is consumed as indicated on analysis by TLC (silica gel). The reaction mixture is quenched with a saturated aq NaHCO: solution (50 mL). Water (50 mL) is then added and the product is extracted with EtOAc (3×100 mL). The organic layers are combined, washed with brine (30 mL) and dried (Na₂SO₄). The solvent is removed under reduced pressure. The resulting solid is purified by flash column chromatography (silica gel) to afford the oxadiazole.

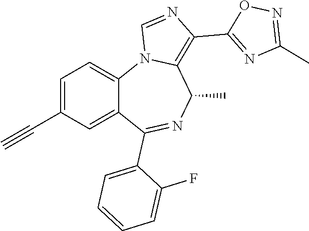 [SMILES: C#Cc1ccc2c(c1)C(c1ccccc1F)=N[C@@H](C)c1c(-c3nc(C)no3)ncn1-2]

(S)-5-(8-Ethynyl-6-(2-fluorophenyl)-4-methyl-4H-benzo[f]imidazo[1,5-a][1,4]diazepin-3-yl)-3-methyl-1,2,4-oxadiazole (GL-1-65). GL-I-65 was prepared from ethyl (S)-8-ethynyl-6-(2-fluorophenyl)-4-methyl-4H-benzo[f]imidazo[1,5-a][1,4]diazepine-3-carboxylate following the general procedure for oxadiazoles with N′-hydroxyacetimidamide (0.154 g, 2.08 mmol). The crude residue was purified by flash column chromatography (silica gel, EtOAc/hexane 3:2) to yield the title compound as a white powder (0.174 g, 85%): mp 225-226° C.; [α]_{D}²⁵=+32.52 (c 2.86, CHCl₃); ¹H NMR (300 MHz, CDCl₃); δ 8.08 (s, 1H), 7.74 (d, J=8.2 Hz, 1H), 7.63 (d, J=8.4 Hz, 2H), 7.48 (dd, J=18.1, 11.6 Hz, 2H), 7.27 (t, J=7.5 Hz, 1H), 7.06 (t, J=9.2 Hz, 1H), 6.75 (q, J=7.1 Hz, 1H), 3.18 (s, 1H), 2.47 (s, 3H), 1.35 (d, J=7.2 Hz, 3H); ¹³C NMR (75 MHz, CDCl₃); δ 170.75 (s), 167.42 (s), 163.40 (s), 160.09 (d, J_{C-F}=250.5 Hz), 139.26 (s), 136.32 (s), 135.34 (s), 134.17 (s), 134.05 (s), 132.06 (d, J_{C-F}=8.5 Hz), 131.20 (s), 129.63 (s), 128.55 (d, J_{C-F}=12.3 Hz), 124.83 (s), 124.50 (d, J_{C-F}=3.2 Hz), 122.21 (s), 121.88 (s), 116.21 (d, J_{C,F}=21.4 Hz), 81.34 (s), 79.95 (s), 50.16 (s), 14.94 (s), 11.66 (s); HRMS (ESI/IT-TOF) m/z: [M +H]⁺ Calcd for C₂₃H₁₇FN₅O 398.1412; found 398.1419.

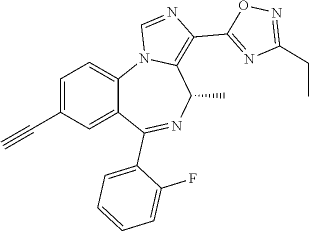 [SMILES: C#Cc1ccc2c(c1)C(c1ccccc1F)=N[C@@H](C)c1c(-c3nc(CC)no3)ncn1-2]

(S)-3-Ethyl-5-(8-ethynyl-6-(2-fluorophenyl)-4-methyl-4H-benzo[f]imidazo[1,5-a][1,4]diazepin-3-yl)-1,2,4-oxadiazole (GL-I-66). GL-I-66 was prepared from ethyl (S)-8-ethynyl-6-(2-fluorophenyl)-4-methyl-4H-benzo[f]imidazo[1,5-a][1,4]diazepine-3-carboxylate following the general procedure for oxadiazoles with N′-hydroxypropionimidamide (0.183 g, 2.08 mmol). The crude residue was purified by flash column chromatography (silica gel, Et(Ac/Hexane 3:2) to yield the title compound as a white powder (0.168 g, 79%): mp 199-200° C.; [α]_{D}²⁵=+50.00 (c 0.22, CHCl₃); ¹H NMR (300 MHz, CDCl₃); δ 8.08 (s, 1H), 7.75 (d, J=8.2 Hz, 1H), 7.63 (d, J=8.3 Hz, 2H), 7.54-7.40 (m, 2H), 7.27 (t, J=7.4 Hz, 1H), 7.06 (t, J=9.2 Hz, 1H), 6.75 (q, J=14.4, 7.2 Hz, 1H), 3.18 (s, 1H), 2.84 (q, J=7.6 Hz, 2H), 1.40 (d, J=7.6 Hz, 3H), 1.36 (t, J=3.6 Hz, 3H), ¹³C NMR (75 MHz, CDCl₃): δ 171.88 (s), 170.70 (s), 163.41 (s), 160.10 (d, J_{C-F}=251.4 Hz), 139.22 (s), 136.26 (s), 135.33 (s), 134.22 (s), 134.07 (s), 132.04 (d, J_{C-F}=8.7 Hz), 131.15 (s), 129.58 (d, J_{C-F}=6.0 Hz), 128.58 (d, J_{C-F}=11.9 Hz), 124.99 (s), 124.51 (d, J_{C-F}=3.2 Hz), 122.18 (s), 121.88 (s), 116.22 (d, J_{C-F}=21.5 Hz), 81.34 (s), 79.90 (s), 50.23 (s), 19.74 (s), 14.97 (s), 11.51 (s); HRMS (ESI/IT-TOF) m/z: [M+H]⁺ Caled for C₂₄H₁₉FN₅O 412.1568; found 412.1569.

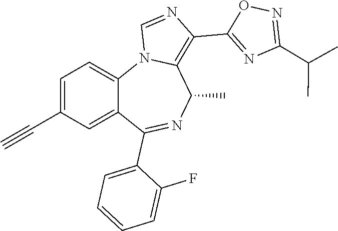 [SMILES: C#Cc1ccc2c(c1)C(c1ccccc1F)=N[C@@H](C)c1c(-c3nc(C(C)C)no3)ncn1-2]

(S)-5-(8-Ethynyl-6-(2-fluorophenyl)-4-methyl-4H-benzo[f]imidazo[1,5-a][1,4]diazepin-3-yl)-3-isopropyl-1,2,4-oxadiazole (GL-1-81). GL-I-81 was prepared from ethyl (S)-8-ethynyl-6-(2-fluorophenyl)-4-methyl-4H-benzo[f]imidazo[1,5-a][1,4]diazepine-3-carboxylate following the general procedure for oxadiazoles with N′-hydroxyisobutyrimidamide (0.212 g, 2.08 mmol). The crude residue was purified by flash column chromatography (silica gel, EtOAc/hexane 3:2) to yield the title compound as a white powder (0.180 g, 82%): mp 205-206° C.; [α]_{D}²⁵=+22.37 (c 0.76, CHCl₃); ¹H NMR (300 MHz, CDCl₃): δ 8.06 (s, 1H), 7.66 (d, J=8.3 Hz, 1H), 7.60 (d, J=8.4 Hz, 1H), 7.53 (t, J=7.3 Hz, 1H), 7.44-7.30 (m, 2H), 7.17 (t, J=7.5 Hz, 1H), 6.96 (t, J=9.2 Hz, 1H), 6.67 (q, J=7.0 Hz, 1H), 3.14 (s, 1H), 3.09 (q, J=7.0 Hz, 1H), 1.32 (d, J=7.0 Hz, 6H), 1.28 (d, J=7.5 Hz, 3H); ¹³C NMR (75 MHz, CDCl3): δ 175.24 (s), 170.61 (s), 163.38 (s), 160.03 (d, J_{C-F}=251.5 Hz), 139.17 (s), 136.34 (s), 135.36 (s), 134.17 (s), 133.98 (s), 132.01 (d, J_{C-F}=8.3 Hz), 131.10 (s), 129.48 (s), 128.58 (d, J_{C-F}=12.3 Hz), 124.96 (s), 124.48 (d, J_{C-F}=3.1 Hz), 122.28 (s), 121.82 (s), 116.16 (d, J_{C-F}=21.4 Hz), 81.30 (s), 80.03 (s), 50.22 (s), 26.70 (s), 20.57 (s), 20.51 (s), 14.94 (s); HRMS (ESI/IT-TOF) m/z: [M+H]⁺ Calcd for C₂₅H₂₁FN₅O 426.1725; found 426.1728.

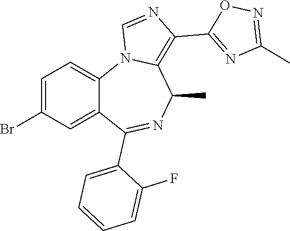 [SMILES: Cc1noc(-c2ncn3c2[C@@H](C)N=C(c2ccccc2F)c2cc(Br)ccc2-3)n1]

(R)-5-(8-Bromo-6-(2-fluorophenyl)-4-methyl-4H-benzo[f]imidazo[1,5-a][1,4] diazepin-3-yl)-3-methyl-1,2,4-oxadiazole (GL-III-60). GL-III-60 was prepared from ethyl (R)-8-bromo-6-(2-fluorophenyl)-4-methyl-4H-benzo[f]imidazo[1,5-a][1,4]diazepine-3-carboxylate (6 g, 13.5 mmol) following the general procedure for oxadiazoles with N′-hydroxyacetimidamide (3 g, 40.5 mmol) and NaH (60% dispersion in mineral oil, 0.8 g, 14.9 mmol). The crude residue was purified by flash column chromatography (silica gel, EtOAc/Hexane 3:2) to yield the title compound as a white powder (5.6 g, 91.8%): ¹H NMR (500 MHz, CDCl₃) δ 8.08 (s, 1H), 7.76 (dd, J=8.5, 1.9 Hz, 1H), 7.61 (t, J=7.0 Hz, 1H), 7.55 (d, J=8.6 Hz, 1H), 7.52-7.42 (m, 2H), 7.26 (td, J=7.6, 0.9 Hz, 1H), 7.11-6.99 (m, 1H), 6.74 (q, J=7.3 Hz, 1H), 2.45 (s, 3H), 1.35 (d, J=7.3 Hz, 3H); ¹³C NMR (126 MHz, CDCl₃) δ 170.69 (s), 167.46 (s), 161.09 (s), 161.06 (d, J=495.1 Hz), 139.12 (s), 136.31 (s), 135.16 (s), 133.37 (s), 133.25 (s), 132.36 (d, J=7.7 Hz), 131.26 (s), 131.02 (s), 128.15 (d, J=11.0 Hz), 124.87 (s), 124.61 (d, J=3.3 Hz), 123.71 (s), 121.26 (s), 116.29 (d, J=21.4 Hz), 50.15 (s), 14.98 (s), 11.69 (s); HRMS (ESI/IT-TOF) m/z: [M+H] Caled for C₂₁H₁₆BrFN₅O 452.0517, found 452.0542.

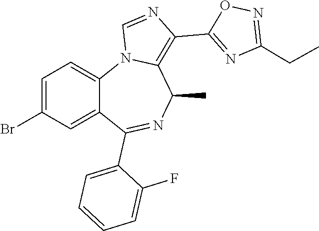 [SMILES: CCc1noc(-c2ncn3c2[C@@H](C)N=C(c2ccccc2F)c2cc(Br)ccc2-3)n1]

(R)-5-(8-Bromo-6-(2-fluorophenyl)-4-methyl-4H-benzo[f]imidazo[1,5-a][1,4] diazepin-3-yl)-3-ethyl-1,2,4-oxadiazole (GL-III-98). GL-III-98 was prepared from ethyl (R)-8-bromo-6-(2-fluorophenyl)-4-methyl-4H-benzo[f]imidazo[1,5-a][1,4]diazepine-3-carboxylate (2.5 g, 5.65 mmol) following the general procedure for oxadiazoles with N′-hydroxypropionimidamide (2.0 g, 22.6 mmol) and NaH (0.25 g, 6.2 mmol). The crude residue was purified by flash column chromatography (silica gel, EtO Ac/Hexane 2:3) to yield the title compound as a white powder (2.2 g, 83.7%): ¹H NMR (300 MHz, CDCl₃) δ 8.05 (s, 1H), 7.71 (d, J=8.6 Hz, 1H), 7.53 (d, J=8.6 Hz, 2H), 7.44-7.31 (m, 2H), 7.19 (t, J=7.5 Hz, 1H), 6.98 (t, J=9.2 Hz, 1H), 6.69 (q, J=7.1 Hz, 1H), 2.77 (q, J=7.6 Hz, 2H), 1.32 (t, J=7.6 Hz, 6H); ¹³C NMR (75 MHz, CDCl₃) δ 171.82 (s), 170.67 (s), 162.84 (s), 160.03 (d, J=250.7 Hz), 139.10 (s), 136.28 (s), 135.07 (s), 133.35 (s), 133.11 (s), 132.18 (d, J=8.0 Hz), 131.16 (s), 131.00 (s), 128.30 (d. J=12.4 Hz), 124.54 (d, J=3.0 Hz), 123.75 (s), 121.13 (s), 116.20 (d, J=21.4 Hz), 50.20 (s), 19.72 (s), 14.95 (s), 11.50 (s); HRMS (ESI/IT-TOF) m/z: [M+H] Caled for C₂₂H₁₈BrFN₅O 468.0655, found 468.0659.

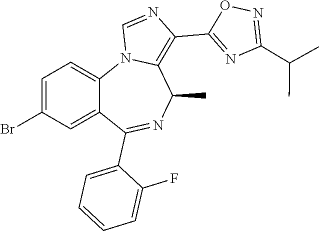 [SMILES: CC(C)c1noc(-c2ncn3c2[C@@H](C)N=C(c2ccccc2F)c2cc(Br)ccc2-3)n1]

(R)-5-(8-Bromo-6-(2-fluorophenyl)-4-methyl-4H-benzo[f]imidazo[1,5-a][1,4] diazepin-3-yl)-3-isopropyl-1,2,4-oxadiazole (GL-IV-01). GL-IV-01 was prepared from ethyl (R)-8-bromo-6-(2-fluorophenyl)-4-methyl-4H-benzo[f]imidazo[1,5-a][1,4]diazepine-3-carboxylate (3.5 g, 7.91 mmol) following the general procedure for oxadiazoles with N′-hydroxyisobutyrimidamide (3.2 g, 31.6 mmol) and NaH (60% dispersion in mineral oil, 0.35 g, 8.7 mmol). The crude residue was purified by flash column chromatography (silica gel, EtOAc/Hexane 1:1) to yield the title compound as a white powder (3.0 g, 78.2%): ¹H NMR (300 MHz, CDCl₃) δ 8.04 (s, 1H), 7.70 (d, J=8.8 Hz, 1H), 7.53 (d, J=8.6 Hz, 2H), 7.43-7.31 (m, 2H), 7.19 (t, J=7.5 Hz, 1H), 6.98 (t, J=9.1 Hz, 1H), 6.69 (q, J=7.0 Hz, 1H), 3.11 (dt, J=13.8, 6.9 Hz, 1H), 1.33 (d, J=7.0 Hz, 6H), 1.31 (d, J=9.3 Hz, 3H); ¹³C NMR (75 MHz, CDCl₃) δ 175.26 (s), 170.58 (s), 162.85 (s), 160.02 (d, J=250.7 Hz), 139.06 (s), 136.26 (s), 135.08 (s), 133.37 (s), 133.11 (s), 132.60 (s), 132.18 (d, J=8.0 Hz), 131.11 (s), 130.96 (s), 128.31 (d, J=12.3 Hz), 124.57 (s), 123.75 (s), 121.11 (s), 116.20 (d, J=21.4 Hz), 50.25 (s), 26.72 (s), 20.59 (s), 20.53 (s), 14.96 (s); HRMS (ESI/IT-TOF) m/z: [M+H] Calcd for C₂₃H₂₀FN₅O 482.0812, found 482.0804.

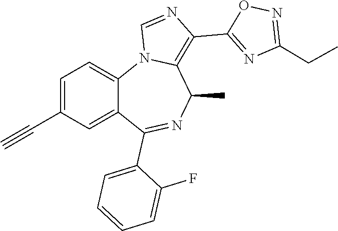 [SMILES: C#Cc1ccc2c(c1)C(c1ccccc1F)=N[C@H](C)c1c(-c3nc(CC)no3)ncn1-2]

(R)-3-ethyl-5-(8-ethynyl-6-(2-fluorophenyl)-4-methyl-4H-benzo[f]imidazo[1,5-a][1,4]diazepin-3-yl)-1,2,4-oxadiazole (MP-IV-005). MP-IV-005 was prepared from ethyl (R)-8-ethynyl-6-(2-fluorophenyl)-4-methyl-4H-benzo[f]imidazo[1,5-a][1,4]diazepine-3-carboxylate (300 mg, 0.774 mmol) following the general procedure for oxadiazoles with N′-hydroxypropionimidamide (273 mg, 3.10 mmol) and NaH (60% dispersion in mineral oil, 77 mg, 1.94 mmol). The crude residue was purified by flash column chromatography (silica gel, EtOAc:hexanes 4:1) to yield the title compound as a white powder (232 mg, 72.8%): ¹H NMR (300 MHz, CDCl₃) δ 8.09 (s, 1H), 7.76 (d, J=7.8 Hz, 1H), 7.63 (d, J=8.2 Hz, 2H), 7.47 (d, J=11.7 Hz, 2H), 7.33-7.24 (m, 1H), 7.06 (t, J=9.1 Hz, 1H), 6.76 (dd, J=14.1, 7.0 Hz, 1H), 3.18 (s, 1H), 2.83 (q, J=7.5 Hz, 2H), 1.40 (dd, J=14.7, 7.1 Hz, 6H); ¹³C NMR (75 MHz, CDCl₃) δ 171.93, 170.51, 161.79, 158.45, 138.81, 136.34, 135.83, 134.44, 134.29, 132.61, 131.37, 125.16, 124.63, 124.58, 122.33, 122.07, 116.46, 116.18, 81.19, 80.11, 49.96, 19.76, 15.07, 11.52; HRMS (LCMS-IT-TOF) Calc. for C24H18FN5O (M+H)+412.1501, found 412.1497.

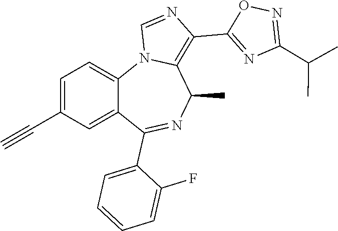 [SMILES: C#Cc1ccc2c(c1)C(c1ccccc1F)=N[C@H](C)c1c(-c3nc(C(C)C)no3)ncn1-2]

(R)-5-(8-ethynyl-6-(2-fluorophenyl)-4-methyl-4H-benzo[f]imidazo[1,5-a][1,4]diazepin-3-yl)-3-isopropyl-1,2,4-oxadiazole (MP-IV-010). MP-IV-010 was prepared from ethyl (R)-8-ethynyl-6-(2-fluorophenyl)-4-methyl-4H-benzo[f]imidazo[1,5-a][1,4]diazepine-3-carboxylate (300 mg, 0.774 mmol) following the general procedure for oxadiazoles with N′-hydroxyisobutyrimidamide (310 mg, 3.10 mmol) and NaH (60% dispersion in mineral oil, 77 mg, 1.94 mmol). The crude residue was purified by flash chromatography (silica gel, EtOAc:hexanes 4:1) to yield the title compound as a white powder (216 mg, 65.7%): ¹H NMR (300 MHz, CDCl₃) δ 8.09 (s, 1H), 7.75 (d, J=7.8 Hz, 1H), 7.63 (d, J=8.3 Hz, 2H), 7.46 (d, J=12.1 Hz, 2H), 7.27 (dd, J=9.4, 5.5 Hz, 1H), 7.06 (t, J=9.2 Hz, 1H), 6.74 (q, J=7.0 Hz, 1H), 3.24-3.10 (m, 2H), 1.38 (t, J=8.3 Hz, 9H); ¹³C NMR (75 MHz, CDCl₃) δ 175.35, 170.27, 161.77, 158.43, 138.51, 136.39, 135.58, 134.70, 134.32, 133.01, 131.47, 128.52, 125.31, 124.67, 122.48, 122.19, 116.52, 116.23, 81.04, 80.31, 49.76, 26.76, 20.59, 20.54, 15.13; HRMS (LCMS-IT-TOF) Calc. for C25H20FN5O (M+H)+ 426.1663, found 426.1659.

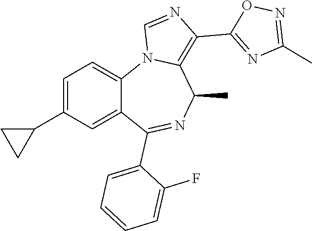 [SMILES: Cc1noc(-c2ncn3c2[C@@H](C)N=C(c2ccccc2F)c2cc(C4CC4)ccc2-3)n1]

(R)-5-(8-Cyclopropyl-6-(2-fluorophenyl)-4-methyl-4H-benzo[f]imidazo[1,5-a][1,4]diazepin-3-yl)-3-methyl-1,2,4-oxadiazole (GL-III-63). To a solution of GL-III-60 (1.6 g, 3.6 mmol) in toluene (100 mL) and water (1.3 mL), cyclopropyl boronic acid (0.55 g, 6.4 mmol), potassium phosphate (3.18 g, 15.0 mmol) and bis(triphenylphosphine)palladium(II) diacetate (0.56 g, 0.75 mmol) were added under argon. A reflux condenser was attached and the mixture was degassed under vacuum with argon; this process was repeated four times. The mixture was stirred and heated to 100° C. After 4 h, the reaction progress was complete on analysis by TLC (silica gel) and it was then cooled to rt. Then water (20 mL) was added and the mixture was extracted with EtOAc (3×25 mL), after which the filtrate was washed with brine (20 mL), dried (Na₂SO₄) and concentrated under reduced pressure. The resulting black residue was purified by column chromatography (silica gel, EtOAc) to afford the desired GL-III-63 as a white solid (1.05 g, 72%): ¹H NMR (300 MHz, CDCl₃) δ 8.03 (s, 1H), 7.59 (t, J=7.5 Hz, 1H), 7.51 (d, J=8.3 Hz, 1H), 7.47-7.36 (m, 1H), 7.24 (dd, J=14.9, 7.4 Hz, 2H), 7.08-6.95 (m, 2H), 6.69 (q, J=7.3 Hz, 1H), 2.44 (s, 3H), 1.87 (dt, J=12.6, 6.8 Hz, 1H), 1.32 (d, J=7.2 Hz, 3H), 1.00 (d, J=8.2 Hz, 2H), 0.64 (dd, J=10.7, 4.7 Hz, 2H); ¹³C NMR (75 MHz, CDCl₃) δ 170.97 (s), 167.38 (s), 161.81 (s), 161.50 (d, J=454.5 Hz), 144.23 (s), 139.19 (s), 136.30 (s), 131.91 (s), 131.80 (s), 131.30 (s), 129.12 (s), 128.99 (s), 128.69 (s), 128.11 (s), 127.32 (s), 124.38 (s), 121.93 (s), 116.06 (d, J=21.8 Hz), 50.09 (s), 15.09 (s), 14.75 (s), 11.67 (s), 9.99 (s); HRMS (ESI/IT-TOF) m/z: [M+H] Calcd for C₂₄H₂₁FN₅O 414.1725, found 414.1728.

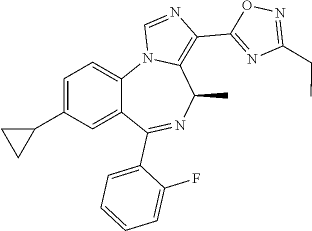 [SMILES: CCc1noc(-c2ncn3c2[C@@H](C)N=C(c2ccccc2F)c2cc(C4CC4)ccc2-3)n1]

(R)-5-(8-Cyclopropyl-6-(2-fluorophenyl)-4-methyl-4H-benzo[f]imidazo[1,5-a][1,4]diazepin-3-yl)-3-ethyl-1,2,4-oxadiazole (GL-IV-03). To a solution of GL-III-98 (1.1 g, 2.38 mmol), tri(O-tolyl)phosphine (85.5 mg, 0.28 mmol), cyclopropyl boronic acid (0.724 g, 8.43 mmol) and potassium phosphate (2.56 g, 12.1 mmol) in toluene (30 mL) and water (0.65 mL), Pd(OAc)₂ (31.5 mg, 0.14 mmol) was added under Ar at rt to form an orange cloudy solution. A reflux condenser was attached. The mixture was allowed to stir at rt for 5 min until the color of the solution turned yellow, as an indication of the formation of the Pd complex generated in situ. The mixture was then placed into a pre-heated oil bath at 100° C. After 2 h the reaction progress was complete on analysis by TLC (silica gel) and it was then cooled to rt. Then water (20 mL) was added and the mixture was extracted with EtOAc (3×25 mL), after which the filtrate was washed with brine (20 mL), dried (Na₂SO₄) and concentrated under reduced pressure. The resulting black residue was purified by column chromatography (silica gel, DCM and 5% MeOH) to afford the desired GL-IV-03 as a white solid (815.5 mg, 81.6%): ¹H NMR (300 MHz, CDCl₃) δ 7.98 (s, 1H), 7.46 (d, J=8.3 Hz, 2H), 7.38-7.25 (m, 1H), 7.13 (t, J=7.5 Hz, 2H), 6.93 (t, J=9.1 Hz, 2H), 6.63 (q, J=7.0 Hz, 1H), 2.73 (q, J=7.6 Hz, 2H), 1.84-1.65 (m, 1H), 1.27 (dd, J=14.1, 6.6 Hz, 6H), 0.90 (d, J=8.3 Hz, 2H), 0.54 (dd, J=10.1, 4.8 Hz, 2H); ¹³C NMR (75 MHz, CDCl₃) δ 171.70 (s), 170.95 (s), 164.32 (s), 160.06 (d, J=250.5 Hz), 144.12 (s), 139.17 (s), 136.28 (s), 131.72 (s), 131.62 (s), 131.17 (s), 129.05 (t, J=6.2 Hz), 128.61 (s), 127.96 (s), 127.20 (s), 124.36 (d, J=8.0 Hz), 122.68 (s), 121.94 (s), 115.94 (d, J=21.6 Hz), 50.13 (s), 19.69 (s), 15.02 (s), 14.68 (s), 11.49 (s), 9.89 (s); HRMS (ESI/IT-TOF) m/z: [M+H] Caled for C₂₅H₂₃FN₅O 428.1881, found 428.1889.

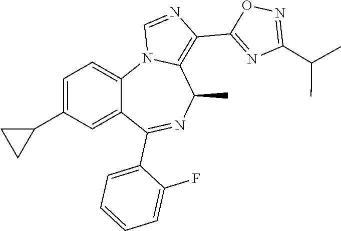 [SMILES: CC(C)c1noc(-c2ncn3c2[C@@H](C)N=C(c2ccccc2F)c2cc(C4CC4)ccc2-3)n1]

(R)-5-(8-Cyclopropyl-6-(2-fluorophenyl)-4-methyl-4H-benzo[f]imidazo[1,5-a][1,4]diazepin-3-yl)-3-isoproyl-1,2,4-oxadiazole (GL-IV-04). To a solution of GL-IV-01 (2.0 g, 4.16 mmol), tri(O-tolyl)phosphine (126.6 mg, 0.416 mmol), cyclopropyl boronic acid (1.07 g, 12.5 mmol) and potassium phosphate (3.8 g, 17.9 mmol) in toluene (40 mL) and water (0.96 mL), Pd(OAc)₂ (46.7 mg, 0.21 mmol) was added under Ar at rt to form an orange cloudy solution. A reflux condenser was attached. The mixture was allowed to stir at rt for 5 min until the color of the solution turned yellow, as an indication for the formation of the Pd complex generated in situ. The mixture was then placed into a pre-heated oil bath at 100° C. After 2.5 h the reaction progress was complete on analysis by TLC (silica gel) and it was then cooled to rt. Then water (20 mL) was added and the mixture was extracted with EtOAc (3×25 mL), after which the filtrate was washed with brine (20 mL), dried (Na₂SO₄) and concentrated under reduced pressure. The resulting black residue was purified by column chromatography (silica gel, DCM and 5% MeOH) to afford GL-IV-04 as a white solid (1.45 g, 78.8%): ^{H NMR (}300 MHz, CDCl₃) δ 8.00 (s, 1H), 7.70-7.43 (m, 1H), 7.42-7.30 (m, 1H), 7.26-7.11 (m, 1H), 6.98 (d, J=13.2 Hz, 1H), 6.65 (q, J=7.1 Hz, 1H), 3.11 (dt, J=13.8, 6.9 Hz, 1H), 1.81 (ddd, J=13.1, 8.4, 5.0 Hz, 1H), 1.31 (dd, J=15.3, 7.1 Hz, 4H), 0.95 (d, J=8.2 Hz, 1H), 0.58 (dd, J=10.3, 4.8 Hz, 1H); ¹³C NMR (75 MHz, CDCl₃) δ 175.19 (s), 170.88 (s), 164.35 (s), 160.09 (d, J=250.5 Hz), 144.11 (s), 139.14 (s), 136.21 (s), 131.80 (s), 131.64 (s), 131.15 (s), 129.32-128.95 (m), 128.65 (s), 128.02 (s), 127.25 (s), 124.47 (d, J=18.9 Hz), 122.68 (s), 121.92 (s), 116.00 (d, J=21.5 Hz), 50.21 (s), 26.71 (s), 20.60 (s), 20.54 (s), 15.04 (s), 14.74 (s), 9.89 (s); HRMS (ESI/IT-TOF) m/z: [M+H] Caled for C₂₆H₂₅FN₅O 442.2038, found 442.2046.

### B. Evaluation of Compounds

Compounds may be analyzed using a number of methods, including ex vivo and in vivo methods.

Radioligand binding assays. Detailed protocols for the primary and secondary radioligand binding assays can be found in the National Institute of Mental Health's Psychoactive Drug Screening Program (NIMH PDSP) Assay Protocol Book (Roth, 2018). Briefly, primary and secondary radioligand binding assays are carried out in a final of volume of 125 μL per well in appropriate binding buffer. The radioactive ligand concentration is close to the Kd ([³H]-U69593 0.83 nM, [³H]-DAMGO 1.20 nM, and [³H]-DADLE 2.69 nM). Total binding and nonspecific binding are determined in the absence and presence of 10 μM of appropriate reference compound (Naltrindole DOR, Salvinorin A KOR and DAMGO MOR). In brief, plates are usually incubated at room temperature and in the dark for 90 min. Reactions are stopped by vacuum filtration onto 0.3% polyethyleneimine (PEI) soaked 96-well filter mats using a 96-well Filtermate harvester, followed by three washes with cold PBS buffer. Scintillation cocktail is then melted onto the microwave-dried filters on a hot plate and radioactivity counted in a Microbeta counter. The data (n=6) were analyzed by nonlinear regression.

### Example 1

### Evaluation of Ligand Affinities for KOR, DOR, MOR, and BZR Receptors

Ligand affinities were determined for KOR (κ opioid receptor), DOR (δ opioid receptor), and MOR (μ opioid receptor) using radioligand binding assays at a screening concentration of 10,000 nM. For these assays, cell homogenates from HEK293 cells that overexpressed KOR, MOR, and DOR were used in combination with [³H]-U69593, [³H]-DAMGO, and [³H]-DADLE, respectively Compounds that exceeded more than 50% radioligand displacement were investigated further in concentration-dependent experiments to determine Kᵢ values. Gamma aminobutyric acid type A receptor (GABA_{A}R) binding was determined with rat brain homogenate and ³H-flunitrazepam. Flunitrazepam binds GABA_{A}R subtypes that consists of two α and two β subunits and one γ or δ subunit. Strong affinities to α_{1-3,5,6}β₁₋₃γ_{1-y}/δ GABA_{A}RS have been reported for flunitrazepam. The extra synaptic GABA_{A}R subtype expression in the brain includes 43% α₁β₂γ₂, 15% α₂β₃γ₂+8% α₂βγ₁, 10% α₃β₃γ₂, 6% α₄βγ/δ, 4% α₅β₃γ₂, and 4% α₆β₂γ₂/δ. Therefore, compounds with high affinity toward the synaptic benzodiazepine-sensitive receptors (BZR) predominately bind α₁₋₃β₂₋₃γ₁₋₂ GABA_{A}Rs. Alternatively, compounds with weak BZR binding might be selective for α₄₋₆βγ/δ GABA_{A}Rs.

Some imidazodiazepines that were developed originally as anxiolytic drug candidates with weak affinity to α₁β₂γ₂ and good affinity to α_{2,3,5}β₃γ₂ GABA_{A}Rs exhibited surprisingly strong KOR affinities. Table 1 summarizes carboxylic acid derivatives of chiral and achiral imidazodiazepines bearing a 2′-fluorophenyl ring.

TABLE 1
Opioid and benzodiazepine receptor binding of 2′-fluorophenyl substituted
imidazodiazepines.

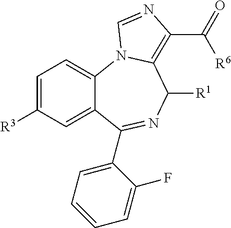

| | | | | R¹ | KOR | MOR | DOR | BZR | KOR | BZR |
|---|---|---|---|---|---|---|---|---|---|---|
| Entry | Compound | R³ | R⁶ | CH₃ | %¹ | %¹ | %¹ | %¹ | (Ki,nM) | (Ki,nM) |
| 1 | GL-I-30 | HC≡C | t-BuO | (S) | 95 | 54 | 7 | 96 | 27 | 177 |
| 2 | GL-I-33 | HC≡C | t-PenO | (S) | 94 | 30 | 25 | 96 | 34 | 117 |
| 3 | GL-I-41 | HC≡C | t-Bu(H)N | (S) | 97 | 40 | 10 | 90 | 39 | 140 |
| 4 | GL-I-78 | c-Pr | EtO | (S) | 96 | 32 | 41 | 76 | 48 | 352 |
| 5 | SH-I-048B | Br | EtO | (S) | 95 | 0 | 0 | 61 | 63 | 96 |
| 6 | GL-I-32 | HC≡C | PrO | (S) | 95 | 9 | 14 | 98 | 64 | 148 |
| 7 | GL-I-31 | HC≡C | i-PrO | (S) | 94 | 32 | 8 | 97 | 65 | 245 |
| 8 | GL-I-38 | HC≡C | c-PrO | (S) | 95 | 18 | 16 | 96 | 68 | 127 |
| 9 | SH-I-047 | Br | EtO | (R) | 82 | 16 | 0 | 84 | 86 | 238 |
| 10 | SH-053-2′F-S-CH₃ | HC≡C | EtO | (S) | 93 | 7 | 36 | 92 | 90 | 111 |
| 11 | GL-I-43 | HC≡C | Et(H)N | (S) | 95 | 22 | 3 | 94 | 102 | 44 |
| 12 | MP-II-023 | HC≡C | Me(H)N | (S) | 91 | 0 | 16 | 97 | 119 | 37 |
| 13 | MP-III-021 | HC≡C | MeO | (S) | 93 | 0 | 22 | 88 | 122 | 219 |
| 14 | GL-I-77 | HC≡C | EtS | (S) | 95 | 41 | 4 | 92 | 125 | 124 |
| 15 | GL-I-55 | HC≡C | c-Pr(H)N | (S) | 93 | 17 | 24 | 95 | 150 | 20 |
| 16 | GL-III-68 | c-Pr | Et(H)N | (R) | 88 | 0 | 10 | 100 | 150 | 452 |
| 17 | GL-III-42 | c-Pr | EtO | (R) | 86 | 0 | 13 | 62 | 174 | 726 |
| 18 | GL-II-74 | HC≡C | Et(H)N | (R) | 86 | 10 | 0 | 84 | 194 | 68 |
| 19 | GL-III-66 | HC≡C | i-Pr(H)N | (R) | 63 | 0 | 0 | 88 | 233 | 271 |
| 20 | MP-III-058 | Br | MeO | (R) | 84 | 0 | 4 | 86 | 237 | 290 |
| 21 | SH-053-2′F-R-CH₃ | HC≡C | EtO | (R) | 89 | 28 | 32 | 85 | 240 | 379 |
| 22 | GL-II-75 | HC≡C | c-Pr(H)N | (R) | 81 | 0 | 3 | 85 | 278 | 93 |
| 23 | GL-II-76 | HC≡C | Pyrrolidine | (R) | 80 | 0 | 0 | 43 | 371 | ?2 |
| 24 | MP-III-022 | HC≡C | Me(H)N | (R) | 80 | 3 | 22 | 95 | 381 | 83 |
| 25 | GL-I-36 | HC≡C | F₃CCH₂O | (S) | 85 | 8 | 0 | 77 | 411 | 418 |
| 26 | GL-III-69 | Br | MezN | (R) | 75 | 0 | 10 | 100 | 511 | 446 |
| 27 | MP-II-075 | HC≡C | BzO | H | 84 | 0 | 16 | 98 | 547 | 21 |
| 28 | MP-III-004 | HC≡C | MeO | (R) | 76 | 0 | 24 | 78 | 599 | 445 |
| 29 | GL-I-54 | HC≡C | Me2N | (S) | 78 | 18 | 11 | 94 | 788 | 90 |
| 30 | GL-III-70 | c-Pr | MezN | (R) | 68 | 0 | 50 | 100 | 800 | 3395 |
| 31 | GL-II-73 | HC≡C | MezN | (R) | 58 | 6 | 0 | 75 | 1189 | 506 |
| 32 | MP-III-019.B | HC≡C | H₂N | (R) | 62 | 2 | 0 | 94 | 1534 | 54 |
| 33 | MP-III-018.B | HC≡C | H₂N | (S) | 51 | 2 | 10 | 97 | 2782 | 17 |
| 34 | GL-III-54 | Cl | HO | (R) | 22 | 0 | 0 | 100 | —² | 42 |
| 35 | SH-053-2′P-S-CH₃ | HC≡C | HO | (S) | 20 | 0 | 18 | 93 | —² | 29 |
| | Acid | | | | | | | | | |
| 36 | SH-053-2'F-R-CH₃- | HC≡C | HO | (R) | 16 | 0 | 0 | 93 | —² | 37 |
| | Acid | | | | | | | | | |
| 37 | GL-II-93 | Br | HO | (R) | 0 | 0 | 0 | 73 | —² | 86 |

¹Percent inhibition at 10,000 nM,
²dose response was carried out only for compounds with an inhibition of >50%

The imidazodiazepine with the highest measured KOR affinity was GL-I-30 (Kᵢ=27 nM) (e.g., Table 1, entry 1). Interestingly, it was the only ligand in the series that also exhibited an appreciable affinity for MOR (Kᵢ=1850 nM). Overall, compounds with a (S) methyl configuration were superior ligands for KOR. The affinity difference between (R) and (S) ligands ranged between 1.3 and 4.9-fold (Table 1, entries 5 vs. 9 and 13 vs. 29). Four different R³ substituents were explored. For entries 4, 5, and 10, cyclopropyl was observed to be superior to bromo and acetylene. Other examples supporting this SAR were entries 20 vs. 28 and 26 vs. 28. For R⁶ (esters, thioesters and amides) a large hydrophobic group like t-butyl demonstrated better KOR binding than smaller substituents such as propyl, ethyl, or methyl (e.g., Table 1, entries 1, 6, 10, and 13). The KOR affinity difference between t-butyl and methyl ester was 4.5-fold. The change from an ethyl to a trifluoroethyl ester reduced KOR affinity by 4.5-fold (Table 1, entries 10 vs. 25). A similar trend was observed for amides. The change from a t-butyl to methyl amide reduced KOR binding by 3.1-fold (e.g., Table 1, entries 3 vs. 12). The thioester GL-I-77 exhibited KOR affinity similar to the affinity of the corresponding ester (e.g., Table 1, entries 10 vs. 14). N,N′-Dimethyl amides were poor KOR ligands. The change from an N-methyl amide to N,N′-dimethyl amide reduced KOR affinity by 6.6-fold (e.g., Table 1, entries 12 vs. 29). Non-substituted amides as well as carboxylic acid ligands exhibited very low KOR affinities. No KOR affinity was observed for GL-II-93 (MIDD0301) (Table 1, entry 37). Interestingly, the BZR affinity for unsubstituted amide and carboxylic acid imidazodiazepines was below 100 nM (e.g., Table 1, entries 32-37). The R³ cyclopropyl group that improved KOR affinity significantly reduced BZR affinity in comparison to a bromo substituent (e.g., Table 1, entries 4 vs. 5, 9 vs. 17 and 26 vs. 30). Similar to KOR, (S)- methyl imidazodiazepines showed better BZR affinities than (R) isomers. In some cases, the affinity difference was 5.6-fold (e.g., Table 1, entries 29 vs. 31).

For the same scaffold, the pendant aromatic ring had a significant impact on KOR binding. The binding of ligands with a 2′-pyridine substituent are summarized in Table 2.

TABLE 2
Opioid and benzodiazepine receptor binding of 2'-pyridine substituted
imidazodiazepines.

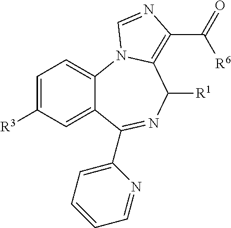

| | | | | R¹ | KOR¹ | MOR¹ | DOR¹ | BZR¹ | KOR | BZR |
|---|---|---|---|---|---|---|---|---|---|---|
| Entry | Name | R³ | R⁶ | CH₃ | % | % | % | % | (Ki,nM) | (Ki,nM) |
| 1 | MP-II-068 | HC≡C | iPrO | H | 85 | 0 | 34 | 97 | 550 | 42 |
| 2 | GL-II-06 | Br | EtO | (R) | 79 | 11 | 9 | 81 | 401 | 556 |
| 3 | Hz-166 | HC≡C | Et0 | H | 60 | 6 | 49 | n.d. | 3821 | n.d. |
| 4 | GL-II-19 | HC≡C | EtO | (R) | 44 | 17 | 2 | 71 | —² | 1143 |
| 5 | MP-III-024 | HC≡C | MeO | H | 43 | 0 | 24 | 81 | —² | 277 |
| 6 | GL-II-32 | HC≡C | MeO | (R) | 35 | 32 | 0 | 63 | —² | 1427 |
| 7 | GL-II-31 | HC≡C | MeHN | (R) | 23 | 17 | 3 | 72 | —² | 1697 |
| 8 | GL-II-51 | Br | HO | (R) | 11 | 0 | 0 | 84 | —² | 181 |
| 9 | SR-II-54 | HC≡C | HO | H | 9 | 8 | 1 | 80 | —² | 69 |
| 10 | GL-II-30 | HC≡C | HO | (R) | 0 | 20 | 4 | 65 | —² | 431 |

¹Percent inhibition at 10,000 nM,
²dose response was carried out only for compounds with an inhibition of >50%,
n.d. = not determined

Compounds with a 2′-pyridine substituent exhibited a lower KOR affinity than those bearing a 2′-fluorophenyl substituent. The difference in the KOR affinity ranged from 2 to 4.7-fold (e.g., Table 2, entry 2 vs. Table 1, entry 9). Similar to the compounds in Table 1, ligands with larger hydrophobic groups were more potent (e.g., ethyl ester GL-II-19 vs. methyl ester GL-II-32, Table 2, entry 4 vs. 6). The carboxylic acid ligands showed weak KOR affinity (e.g., Table 2, entries 8-10). Achiral imidazodiazepines, such as HZ-166 and MP-III-024, exhibited slightly stronger affinities to KOR than their chiral counterparts with (R) configurations (e.g., Table 2, entries 3 vs. 4 and 5 vs. 6). For substitutions at R³, compounds with a bromo function were more active than those with an acetylene group (e.g., Table 2, entries 2 vs. 4 and 8 vs 10). In contrast, 2′-pyridine bearing ligands exhibited good to excellent BZR binding. Compounds with achiral ligands (e.g., MP-II-68 and SR-II-54—Table 2, entries 1 and 9) were especially active. Moreover, in this series, carboxylic acid derivatives were good BZR ligands (Table 2, entries 8-10).

The ester and amide bioisosteres were investigated next. The binding for a series of oxadiazoles is summarized in Table 3.

TABLE 3
Opioid and benzodiazepine receptor binding of imidazodiazepine oxadiazoles.

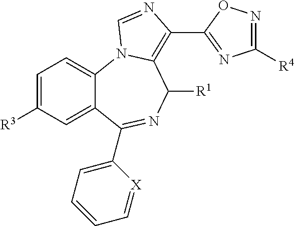

| | | | | | | KOR¹ | MOR¹ | DOR¹ | BZR¹ | KOR | BZR |
|---|---|---|---|---|---|---|---|---|---|---|---|
| Entry | Name | R³ | R⁴ | R¹ | X | % | % | | % | (Ki,nM) | (Ki,nM) |
| 1 | GL-I-81 | HC≡C | iPr | (S) | C—F | 93 | 64 | 25 | 83 | 127 | 839 |
| 2 | MP-IV-010 | HC≡C | iPr | (R) | C—F | 94 | 33 | 5 | 64 | 145 | 814 |
| 3 | GL-I-66 | HC≡C | Et | (S) | C—F | 92 | 28 | 2 | 80 | 212 | 654 |
| 4 | GL-I-65 | HC≡C | Me | (S) | C—F | 88 | 2 | 7 | 79 | 222 | 1147 |
| 5 | GL-IV-03 | c-Pr | Et | (R) | C—F | 91 | 25 | 67 | 65 | 232 | n.d. |
| 6 | GL-III-60 | Br | Me | (R) | C—F | 87 | 26 | 18 | 100 | 232 | 784 |
| 7 | MP-IV-004 | HC≡C | Me | (R) | C—F | 86 | 7 | 10 | 63 | 444 | 1079 |
| 8 | GL-II-54 | HC≡C | Et | (R) | N | 80 | 0 | 0 | 66 | 504 | 2037 |
| 9 | GL-III-63 | c-Pr | Me | (R) | C—F | 78 | 8 | 31 | 28 | 678 | —2 |
| 10 | GL-III-64 | c-Pr | Me | (R) | N | 60 | 7 | 14 | 92 | 2048 | 1490 |
| 11 | MP-III-085 | HC≡C | Me | H | N | 40 | 0 | 0 | 82 | —² | 357 |
| 12 | MP-III-080 | HC≡C | Et | H | N | 47 | 0 | 0 | 88 | —² | 303 |

¹Percent inhibition at 10,000 nM,
²dose response was carried out only for compounds with an inhibition of >50%,
n.d. = not determined

The imidazodiazepine with this strongest KOR affinity in this series was GL-I-81 (Table 3, entry 1). Additionally, GL-I-81 was the only ligand in this series that exhibited appreciable MOR affinity (Kᵢ=2920 nM). Interestingly, imidazodiazepine oxadiazoles exhibited SAR similar to imidazodiazepine esters and amides. 2′-Fluorophenyl substituted compounds demonstrated better KOR affinities than corresponding 2′-pyridine ligands (e.g., Table 3, entries 9 vs. 10). Isopropyl substituted oxadiazoles were more active than the corresponding ethyl or methyl substituted imidazodiazepine oxadiazoles (e.g., Table 3, entries 1, 3 and 4). No significant difference in KOR affinity was observed between the methyl and ethyl substitution (e.g., Table 2, entries 3 vs. 4, 6 vs. 7 and 8 vs. 9). Compounds with a (S) methyl configuration exhibited better KOR affinities than the corresponding (R) ligands (e.g., Table 3, entries 1 vs. 2 and 4 vs. 7). For R³ substitution, bromo was superior to acetylene and cyclopropyl (e.g., Table 2, entries 6, 7, and 9). All oxadiazoles exhibited better affinities towards KOR than BZR, except achiral ligands with a 2′-pyridine substituent (Table 2, entries 11 and 12).

Next, oxazole imidazodiazepines were explored. Their binding is summarized in Table 4.

TABLE 4
Opioid and benzodiazepine receptor binding of imidazodiazepine oxazoles.

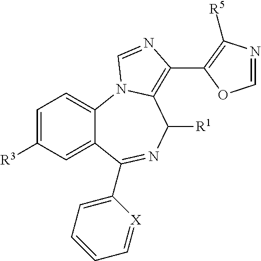

| | | | | | | KOR¹ | MOR¹ | DOR¹ | BZR¹ | KOR | BZR |
|---|---|---|---|---|---|---|---|---|---|---|---|
| Entry | Name | R³ | R⁵ | R¹ | X | % | % | % | % | (Ki,nM) | (Ki,nM) |
| 1 | SH-I-85 | Br | H | H | C—F | 91 | 16 | 33 | 99 | 162 | 11 |
| 2 | GL-III-76 | Br | H | (S) | C—F | 83 | 3 | 0 | 100 | 221 | 120 |
| 3 | GL-III-36 | Br | H | (R) | C—F | 88 | 0 | 12 | 86 | 246 | 319 |
| 4 | KRM-II-18B | HC≡C | H | H | C—F | 89 | 11 | 20 | 97 | 361 | 38 |
| 5 | KRM-III-59 | Br | Me | H | C—F | 73 | 0 | 11 | 96 | 408 | 20 |
| 6 | GL-III-73 | HC≡C | H | (R) | C—F | 74 | 12 | 4 | 65 | 449 | 602 |
| 7 | GL-III-78 | HC≡C | H | (S) | C—F | 74 | 10 | 9 | 101 | 451 | 26} |
| 8 | KRM-II-73 | Br | H | H | C—H | 83 | 11 | 32 | 97 | 736 | 45 |
| 9 | KRM-III-66 | Br | Me | H | C—H | 67 | 12 | 11 | 86 | 756 | 49 |
| 10 | KRM-III-65 | HC≡C | Me | H | C—F | 64 | 0 | 16 | 96 | 879 | 45 |
| 11 | KRM-II-82 | HC≡C | H | H | C—H | 78 | 5 | 20 | 95 | 1203 | 96 |
| 12 | KRM-II-97 | Br | H | H | N | 62 | 18 | 8 | 93 | 1959 | 140 |
| 13 | KRM-III-67 | HC≡C | H | H | N | 48 | 30 | 3 | 71 | —² | 144 |
| 14 | KRM-II-81 | Br | Me | H | N | 46 | 7 | 15 | 86 | —² | 294 |
| 15 | KRM-III-79 | HC≡C | Me | H | N | 9 | 32 | 1 | 81 | —² | 308 |

¹Percent inhibition at 10,000 nM,
²dose response was carried out only for compounds with an inhibition of >50%

Imidazodiazepine oxazoles with a bromo substituent in the R³ position were superior KOR ligands in comparison to those bearing an acetylene group (e.g., Table 4, entries 1 vs. 4, 2 vs. 7, 3 vs. 6, 5 vs. 9, 8 vs. 11, 12 vs. 13 and 14 vs. 15). Ligands with a methyl substituted oxazole were less active than those with a non-substituted oxazole (e.g., Table 4, entries 1 vs. 5, 8 vs. 9, 4 vs. 10, 12 vs. 14, and 13 vs. 15). Also like all other scaffolds, compounds with a 2′-fluorophenyl group exhibited greater KOR affinities than those with phenyl or 2′-pyridine substitutions (Table 4, entries 1, 8 and 12, and entries 5, 9 and 14). The achiral oxazole ligands exhibited better affinities toward KOR than chiral ligands (e.g., Table 4, entries 1 vs. 2 and 4 vs. 6). The configuration of the R¹ methyl group did not significantly influence KOR binding (e.g., Table 4, entries 2 vs. 3 and 6 vs. 7). Achiral oxazoles exhibited better BZR affinities than chiral oxazoles, especially those bearing phenyl or 2′-fluorophenyl substitutions. The chiral imidazodiazepine oxazoles with an (S) configuration were better ligands for BZR than their corresponding (R) isomers (e.g., Table 4, entries 2 vs. 3 and 6 vs. 7). Imidazodiazepines with a methyl substituted oxazole exhibited slightly lower BZR affinities than those without. Finally, 2′-pyridine substitution resulted in lower BZR affinities for imidazodiazepine oxazoles.

### Example 2

### BRET Recruitment Assay with GL-I-30 to Assess KOR Agonism

The BRET recruitment assays were performed in HEK293T cells according to previously reported procedures with minor modifications. Briefly, the cells were co-transfected overnight with Gα_{oA}-RLuc, Gβ3, Gγ8-GFP2 and human KOR receptor at a 1:1:1:1 ratio. The next day, cells were seeded (˜40,000 cells/well) into poly-L-Lysine coated 96-well white clear bottom cell culture plates in DMEM containing 1% dialyzed FBS. 24 hours later, the 96-well plates bottom were covered with white backing (PerkinElmer) and the culture medium was removed. Immediately, the cells were washed with 80 μL/well of assay buffer (1× HBSS, 20 mM HEPES, 1 mg/ml BSA, pH 7.4). Then the cells were treated with 80 μL of drugs in assay buffer for 10 min at room temperature, follow by addition of 20 μL/well of RLuc substrate and incubated for another 10 min. Plates were read in a Mithras LB940 reader for the RLuc Luminescence (400 nm) and GFP2 (515 nm) emission and the ratio of GFP2/RLuc (n=24) was analyzed by non-linear regression using GraphPad Prism 8.

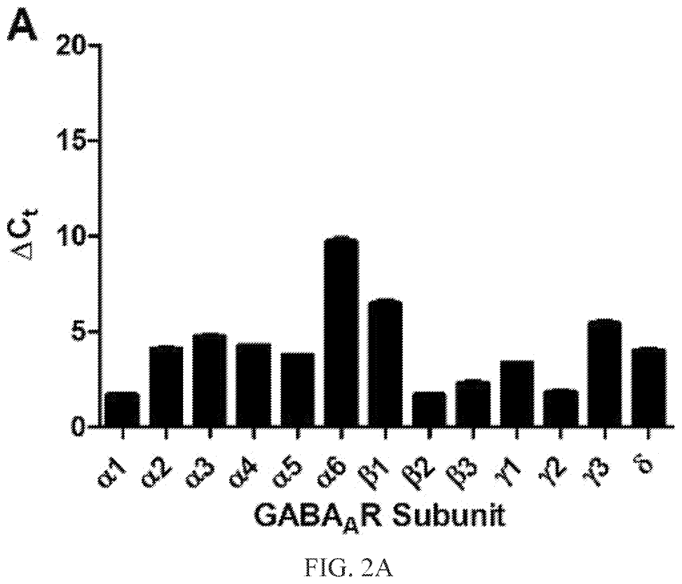

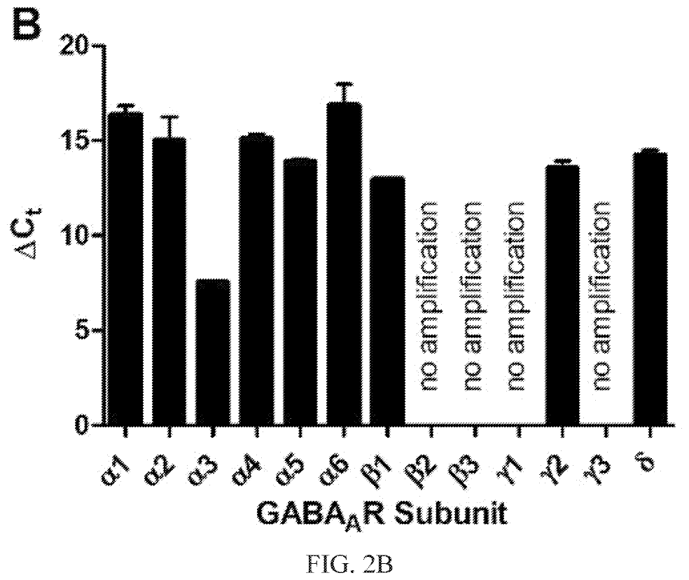

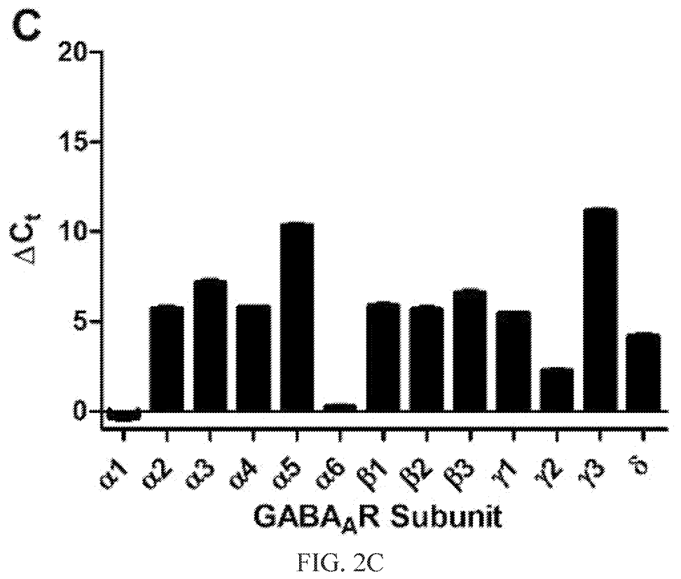

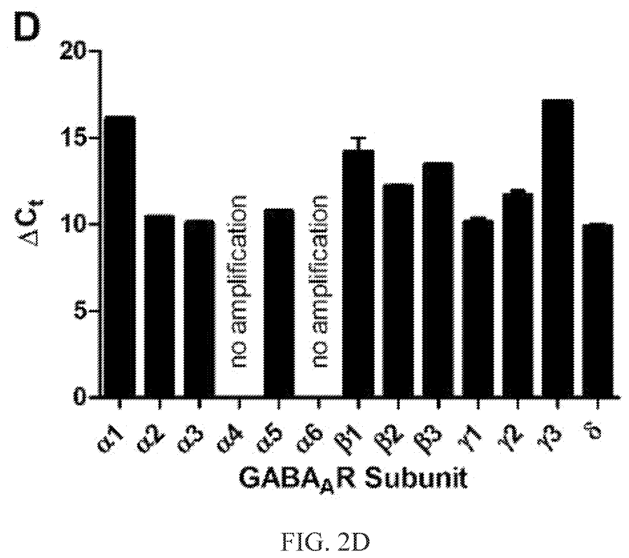

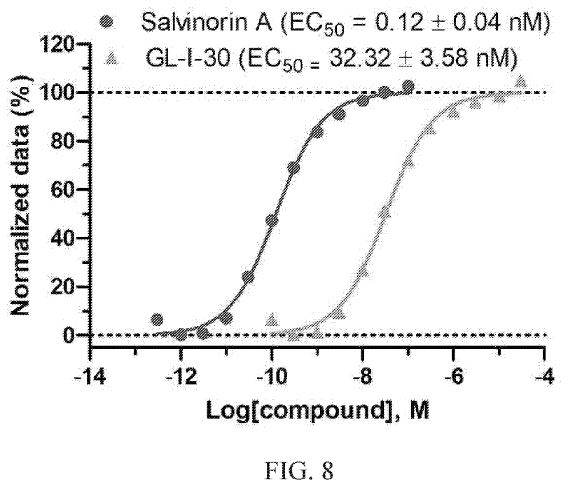

GL-I-30 induced the recruitment of GooA protein to KOR with an EC₅₀ of 32.3 nM (FIG. 2). The efficacy was 100% in comparison to full agonist salvinorin A (FIG. 8). This experimental result establishes that GL-I-30 demonstrates full KOR agonism.

### Example 3

### Evaluation of Ligand Library for Induced NO Inhibition

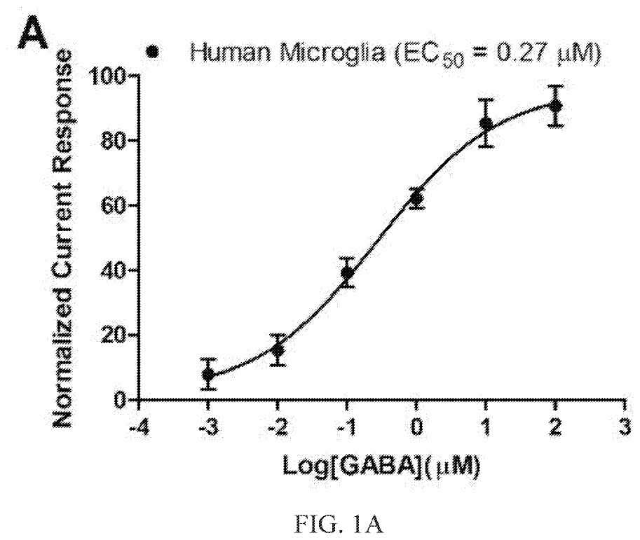

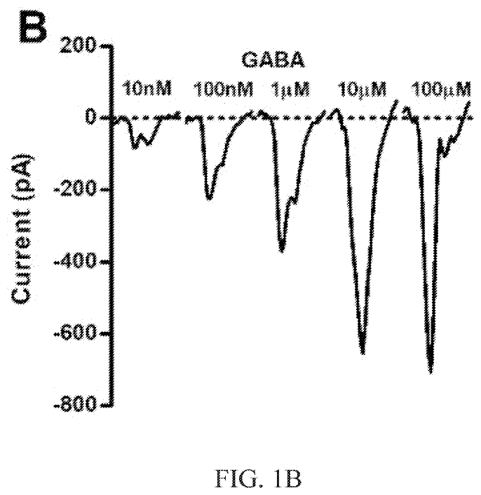

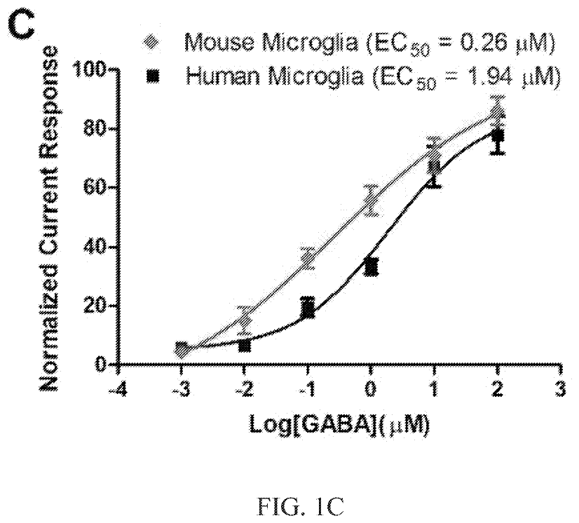

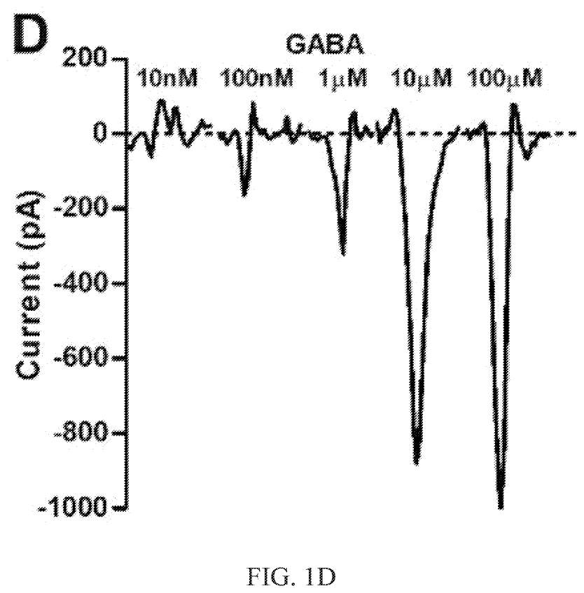

To verify the expression of functional GABA_{A}R on immortalized microglia, cells were patch clamped at −80 mV and current responses acquired for 3 second applications of increasing concentrations of GABA (FIG. 1A-1D). Commercially available HMC3 cells exhibited a robust current change in the presence GABA (FIG. 1B). During the GABA application, the negative current increased followed by a rapid recovery once GABA was washed away with external cellular solution Automated patch clamp technology enabled recording of the average signal of twenty patch clamped cells simultaneously and conducting experiments with eight independent microfluidic systems on the same plate. The time dependent currents resulted an EC₅₀ value of 270 nM for GABA (FIG. 1A). Literature reports have called into question if HMC3 cells (also called CHME-5 cells) are truly of human origin. Accordingly, additional immortalized human and mouse microglia cell lines were obtained from Case Western Reserve University (Cleveland, OH). Both cell lines were cultured and analyzed with patch clamp as described for HMC3 cells (FIG. 1C-1D). The maximum current response for 1 mM GABA was 1245 pA for the human and 1514 pA for the mouse cells. GABA concentration dependent studies established an EC₅₀ of 260 nM and 1940 nM for the mouse and human microglia, respectively (FIG. 1C).

RT-PCR was used to determine the expression of GABA_{A}R subunits in human and mouse microglia (FIGS. 2B and 2D), which was compared to corresponding mRNA levels in mouse cerebellum and human brain extract (FIGS. 2A and 2C). Overall, the expression of GABA_{A}R subunits is less pronounced in human microglia than in an extract containing all brain cell types (FIG. 2A-2B). For alpha subunits, α₃ is the most abundant subunit in human microglia in contrast to α₁ in the brain (mostly neurons) (FIG. 2A). Possible binding partners that form functional GABA_{A}R receptors in human microglia are β₁ and γ₂/δ (FIG. 2B). Mouse microglia did not express α₄ or α₆ GABA_{A}R subunits, however, α₂, α₃, and α₅ mRNA were observed at similar amounts (FIG. 2D). Other expressed GABA_{A}R subunits were β₁₋₃, γ₁₋₂ and δ. Thus, mouse microglia express a more diverse subset of GABA_{A}Rs than human microglia. This finding supports the earlier observation that GABA induced a current change at a lower concentration in mouse microglia than human microglia.

Next, a library of GABA_{A}R ligands was investigated to identify compounds that reduce the production of NO as a marker of anti-inflammatory activity. Mouse microglia and macrophage line RAW264.7 were activated with interferon gamma (IFNγ) and E. coli lipopolysaccharide (LPS), followed by compound exposure at 50 or 10 μM for 24 hours in the presence of 1 μM GABA. NO was quantified with a Greiss assay. Cell viability was determined with CellTiter-Glo. The results are summarized in Table 5.

TABLE 5
GABA_{A}R ligand induced NO inhibition and cytotoxicity

| | Inhibition of NO production (%) | | | | Cell viability (%) | | | |
|---|---|---|---|---|---|---|---|---|
| | RAW | | Mouse | | RAW | Mouse | | |
| | macrophages | | microglia | | macrophages | microglia | HEK293^{b} | HEPG2^{b} |
| Compound^{a} | 50 μM | 10 μM | 50 μM | 10 μM | 50 μM | 50 μM | 120 μM | 120 μM |
| GL-I-48 | | 4 ± 2 | | 0 ± 4 | 100 ± 12 | 78 ± 5 | 65 ± 8 | 100 ± 4 |
| GL-I-50 | | 0 ± 1 | | 33 ± 6 | 100 ± 9 | 88 ± 9 | 97 ± 12 | 100 ± 2 |
| GL-I-62 | | 3 ± 2 | | 13 ± 7 | 100 ± 3 | 92 ± 7 | 79 ± 7 | 89 ± 10 |
| GL-I-64 | | 3 ± 3 | | 24 ± 3 | 100 ± 5 | 81 ± 5 | 77 ± 13 | 100 ± 9 |
| GL-I-65 | | | 19 ± 9 | | | 100 ± 12 | | |
| GL-I-66 | | | 86 ± 11 | 5 ± 2 | | 71 ± 9 | | |
| GL-I-81 | 6 ± 2 | 0 ± 2 | 87 ± 2 | 64 ± 9 | 91 ± 7 | 94 ± 10 | | |
| GL-II-05 | | 0 ± 1 | | 12 ± 5 | 100 ± 11 | 88 ± 8 | 94 ± 3 | 92 ± 5 |
| GL-II-06 | | 4 ± 2 | | 14 ± 3 | 100 ± 10 | 87 ± 13 | 83 ± 13 | 89 ± 5 |
| GL-II-18 | | 11 ± 4 | | 49 ± 10 | 99 ± 2 | 89 ± 9 | 53 ± 10 | 100 ± 8 |
| GL-II-19 | | 2 ± 2 | | 32 ± 4 | | 100 ± 11 | 90 ± 5 | 100 ± 9 |
| GL-II-32 | | 6 ± 3 | | 7 ± 2 | 100 ± 4 | 95 ± 8 | 80 ± 6 | |
| GL-II-33 | | | 0 ± 3 | | 100 ± 7 | 100 ± 4 | | |
| GL-II-51 | | 0 ± 1 | | 9 ± 4 | 100 ± 3 | 95 ± 2 | 91 ± 4 | 100 ± 16 |
| GL-II-54 | | | 0 ± 4 | | 100 ± 8 | 95 ± 3 | | |
| GL-II-61 | | 10 ± 4 | | 28 ± 2 | 100 ± 8 | 91 ± 8 | 83 ± 10 | 82 ± 3 |
| GL-II-79 | | 0 ± 3 | | 44 ± 6 | 100 ± 3 | 84 ± 5 | 91 ± 13 | 100 ± 7 |
| GL-II-80 | | 5 ± 3 | | 38 ± 8 | 100 ± 2 | 85 ± 1 | 76 ± 8 | 100 ± 9 |
| GL-III-13 | | 0 ± 2 | | | 100 ± 9 | 92 ± 9 | 74 ± 5 | 100 ± 10 |
| GL-III-23 | | | 28 ± 5 | | | 100 ± 2 | 65 ± 5 | 90 ± 13 |
| GL-III-24 | | 68 ± 8 | | 67 ± 12 | 100 ± 12 | 98 ± 5 | 78 ± 6 | 91 ± 12 |
| GL-III-25 | | 38 ± 9 | | 25 ± 10 | 100 ± 8 | 98 ± 4 | 93 ± 4 | 100 ± 5 |
| GL-III-27 | | 1 ± 3 | | 29 ± 8 | 100 ± 4 | 86 ± 8 | 91 ± 2 | 77 ± 7 |
| GL-III-35 | | 0 ± 4 | | 17 ± 7 | 100 ± 2 | 100 ± 8 | 79 ± 8 | 98 ± 8 |
| GL-III-36 | 58 ± 9 | 0 ± 3 | 41 ± 7 | 8 ± 5 | 81 ± 5 | 97 ± 9 | 80 ± 9 | 100 ± 9 |
| GL-III-42 | | 0 ± 5 | | 55 ± 12 | 100 ± 6 | 82 ± 10 | 62 ± 11 | 100 ± 8 |
| GL-III-52 | | 2 ± 2 | | 57 ± 11 | 97 ± 8 | 85 ± 8 | 73 ± 8 | 81 ± 9 |
| GL-III-53 | | 3 ± 2 | | 85 ± 14 | 97 ± 7 | 72 ± 7 | 47 ± 10 | 94 ± 10 |
| GL-III-54 | | 6 ± 4 | | 31 ± 9 | 99 ± 12 | 82 ± 6 | 96 ± 12 | 93 ± 4 |
| GL-III-58 | | | | 48 ± 4 | 100 ± 14 | 83 ± 4 | 89 ± 2 | 100 ± 5 |
| GL-III-59 | | 8 ± 1 | | 43 ± 7 | 100 ± 3 | 96 ± 4 | 100 ± 4 | 100 ± 2 |
| GL-III-60 | 39 ± 8 | 0 ± 1 | 68 ± 8 | 55 ± 8 | 86 ± 12 | 100 ± 9 | 64 ± 6 | 100 ± 11 |
| GL-III-63 | 62 ± 12 | 13 ± 4 | 89 ± 12 | 25 ± 4 | 87 ± 15 | 100 ± 8 | 18 ± 4 | 16 ± 6 |
| GL-III-64 | | | 26 ± 10 | | | 96 ± 10 | 75 ± 8 | 75 ± 10 |
| GL-III-66 | | 6 ± 4 | | 13 ± 2 | 100 ± 9 | 85 ± 15 | 46 ± 4 | 86 ± 8 |
| GL-III-67 | | 4 ± 2 | | 33 ± 1 | 100 ± 7 | 85 ± 10 | 66 ± 6 | 100 ± 9 |
| GL-III-68 | | 5 ± 1 | | 24 ± 5 | 100 ± 8 | 83 ± 3 | 56 ± 11 | 83 ± 2 |
| GL-III-69 | | 0 ± 1 | | 37 ± 6 | 98 ± 10 | 87 ± 6 | 73 ± 10 | 88 ± 5 |
| GL-III-70 | | 2 ± 2 | | 32 ± 4 | 100 ± 2 | 86 ± 2 | 86 ± 12 | 100 ± 5 |
| GL-III-72 | | 57 ± 7 | | 21 ± 7 | 77 ± 4 | 100 ± 10 | 64 ± 8 | 100 ± 3 |
| GL-III-73 | | 1 ± 6 | | 40 ± 3 | 100 ± 4 | 80 ± 9 | 86 ± 6 | 100 ± 9 |
| GL-III-75 | | 3 ± 5 | | 44 ± 2 | 100 ± 2 | 87 ± 7 | 73 ± 2 | 74 ± 6 |
| GL-III-76 | | 0 ± 3 | | 34 ± 1 | 100 ± 5 | 80 ± 2 | 59 ± 4 | 90 ± 6 |
| GL-III-76A | | 6 ± 3 | | 58 ± 9 | 97 ± 4 | 100 ± 9 | 0 ± 3 | 0 ± 2 |
| GL-III-77 | | 29 ± 2 | | 31 ± 2 | 85 ± 3 | 98 ± 4 | 0 ± 2 | 0 ± 4 |
| GL-III-78 | | 0 ± 6 | | 39 ± 5 | 97 ± 2 | 79 ± 6 | 56 ± 8 | 100 ± 15 |
| GL-III-84 | | 10 ± 5 | | 32 ± 4 | 100 ± 12 | 96 ± 8 | 64 ± 5 | 100 ± 10 |
| GL-III-85 | | 0 ± 2 | | 34 ± 8 | 100 ± 10 | 84 ± 12 | 70 ± 12 | 100 ± 11 |
| GL-III-86 | | 7 ± 4 | | 39 ± 7 | 100 ± 14 | 99 ± 15 | 66 ± 10 | 100 ± 8 |
| GL-III-87 | | 0 ± 1 | | 23 ± 7 | 100 ± 7 | 100 ± 2 | 90 ± 10 | 100 ± 5 |
| GL-III-97 | | 6 ± 1 | | 11 ± 6 | 98 ± 3 | 100 ± 4 | 54 ± 9 | 100 ± 12 |
| GL-III-98 | | 1 ± 4 | | 75 ± 9 | 100 ± 11 | 83 ± 6 | 42 ± 8 | 56 ± 5 |
| GL-III-99 | | 19 ± 3 | | 49 ± 5 | 100 ± 9 | 95 ± 9 | 87 ± 11 | 93 ± 7 |
| GL-IV-01 | | 17 ± 7 | | 56 ± 11 | 100 ± 9 | 100 ± 9 | 72 ± 8 | 100 ± 9 |
| GL-IV-03 | | 75 ± 12 | | 90 ± 10 | 97 ± 7 | 100 ± 5 | 47 ± 5 | 39 ± 5 |
| GL-IV-04 | | 11 ± 2 | | 62 ± 8 | 100 ± 7 | 86 ± 6 | 71 ± 6 | 100 ± 6 |
| GL-IV-05 | | 14 ± 3 | | 59 ± 4 | 100 ± 6 | 88 ± 12 | 47 ± 3 | 100 ± 10 |
| GL-IV-17 | | 5 ± 5 | | 41 ± 5 | 100 ± 5 | 89 ± 9 | 67 ± 5 | 93 ± 8 |
| GL-IV-18 | | 10 ± 7 | | 63 ± 9 | 98 ± 5 | 81 ± 10 | 88 ± 5 | 80 ± 14 |
| DMH-D-053 | | 13 ± 2 | | | 100 ± 8 | 93 ± 13 | 97 ± 10 | 100 ± 11 |
| KRM-II-18B | | | 0 ± 1 | | | 100 ± 3 | | |
| KRM-II-81 | | | 0 ± 1 | | | 100 ± 5 | 96 ± 11 | 100 ± 12 |
| KRM-II-82 | | | 0 ± 3 | | | 100 ± 6 | | |
| MP-III-022 | 51 ± 7 | 9 ± 5 | 60 ± 12 | 36 ± 6 | 100 ± 11 | 86 ± 2 | 75 ± 13 | 94 ± 7 |
| MP-III-080 | | | 0 ± 3 | | | 100 ± 14 | | |
| MP-IV-004 | 51 ± 7 | 0 ± 1 | 60 ± 6 | 34 ± 5 | 83 ± 12 | 100 ± 5 | 40 ± 8 | 100 ± 4 |
| MP-IV-005 | 59 ± 9 | 5 ± 3 | 89 ± 17 | 45 ± 3 | 86 ± 14 | 86 ± 13 | 68 ± 14 | 98 ± 8 |
| MP-IV-010 | 63 ± 11 | 10 ± 2 | 96 ± 13 | 66 ± 13 | 81 ± 10 | 91 ± 10 | 41 ± 4 | 45 ± 7 |
| QH-II-66 | | 17 ± 6 | | 54 ± 9 | 100 ± 12 | 97 ± 11 | 81 ± 2 | 100 ± 4 |
| YT-III-271 | | | 0 ± 4 | | | 100 ± 3 | | |
| YT-III-31 | | | 0 ± 2 | | | 100 ± 8 | 96 ± 10 | 100 ± 7 |

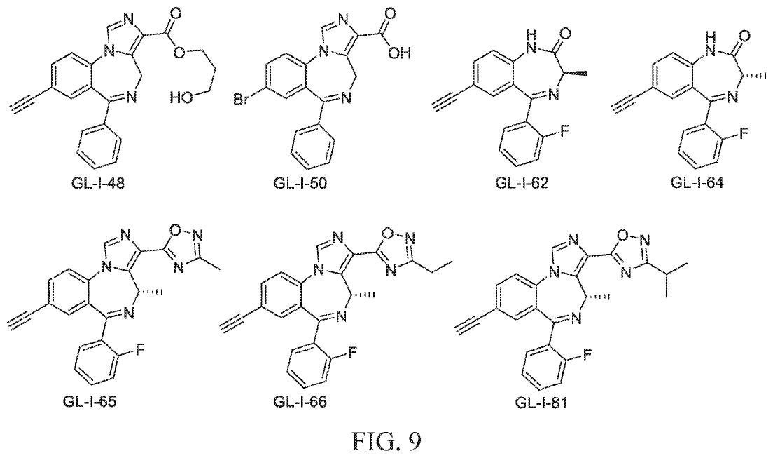

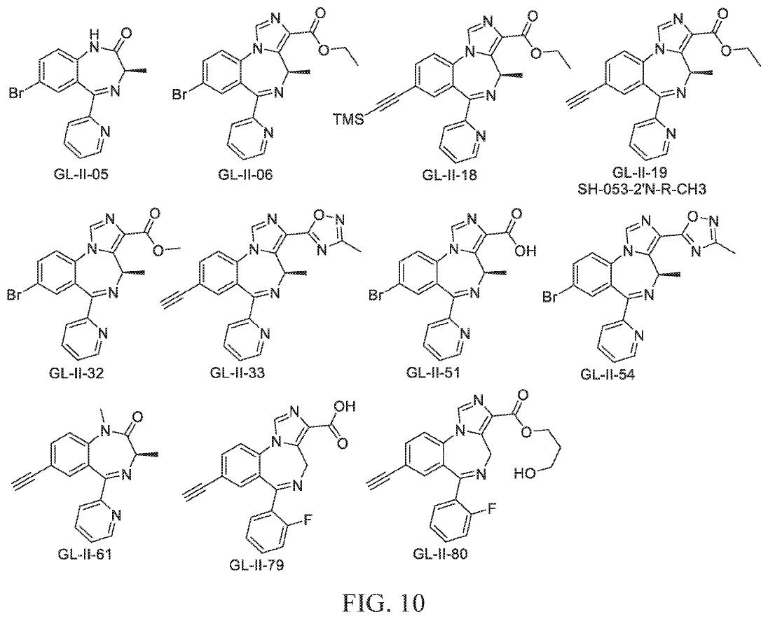

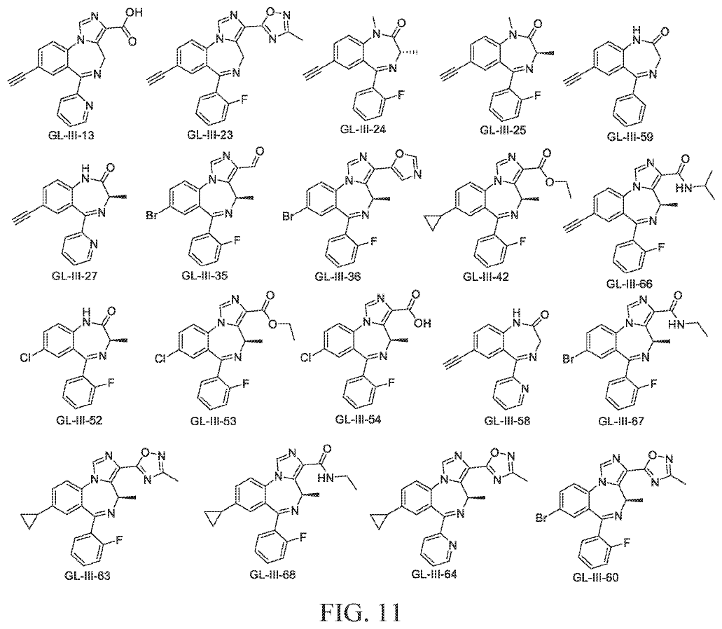

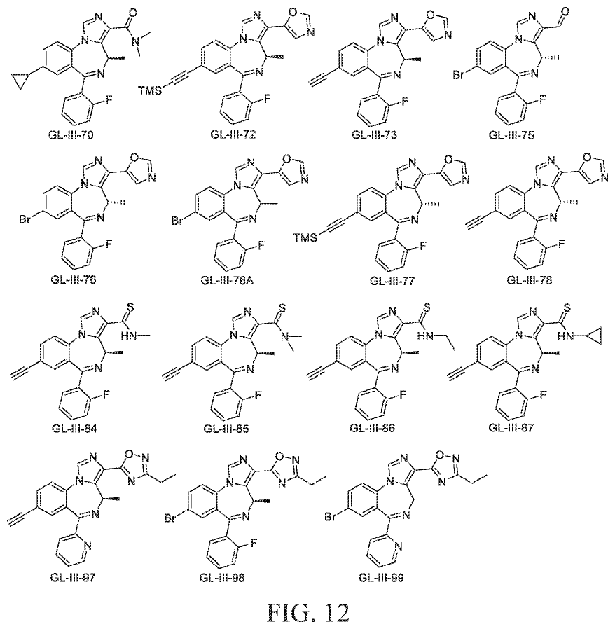

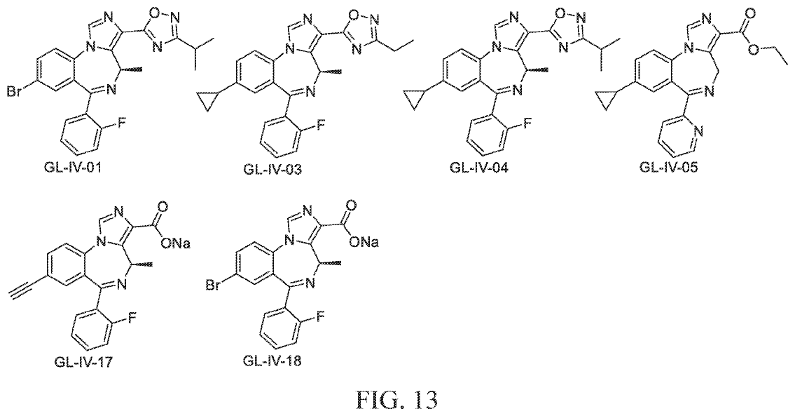

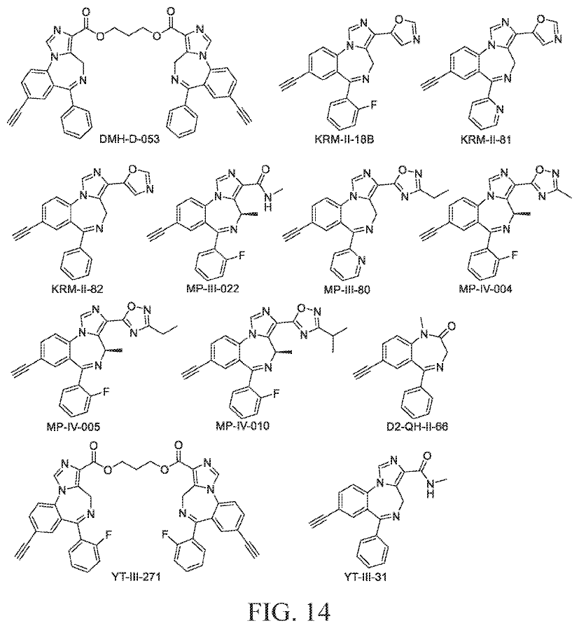

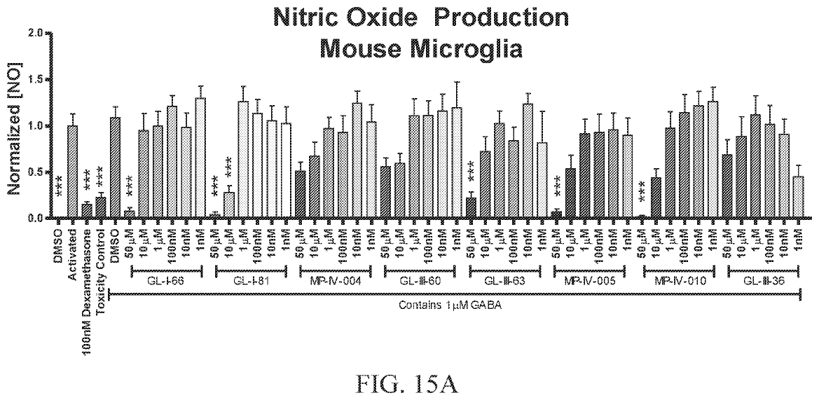

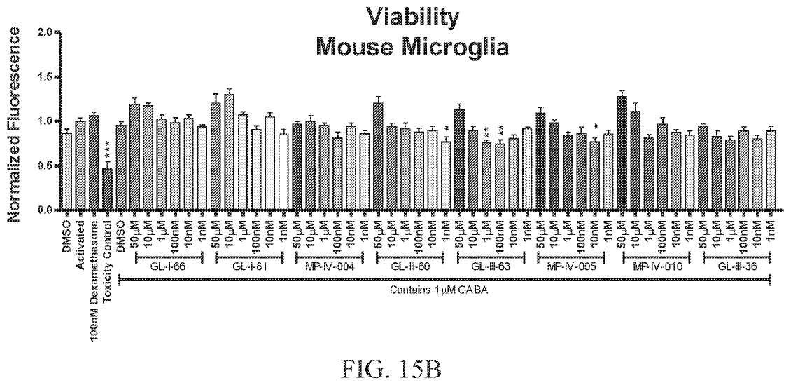

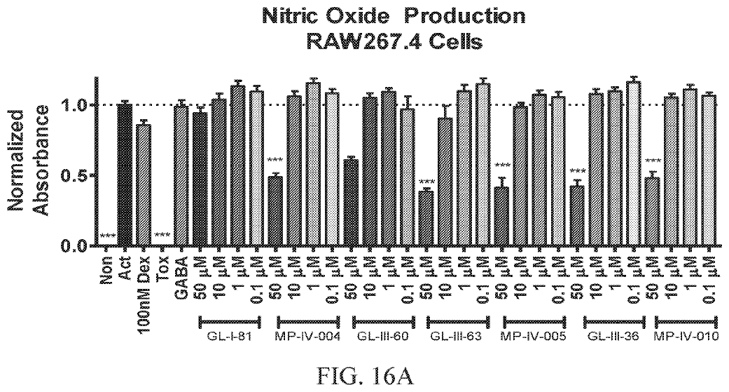

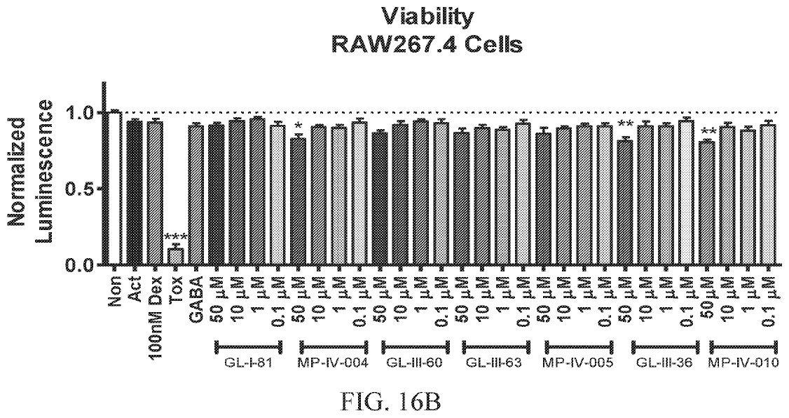

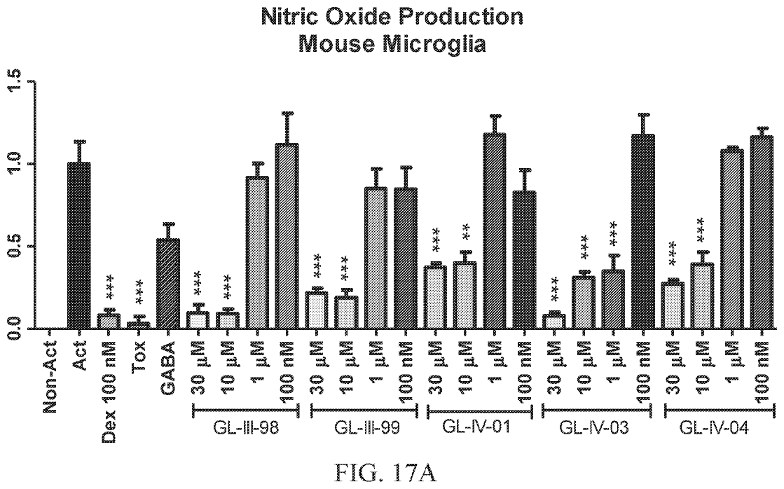

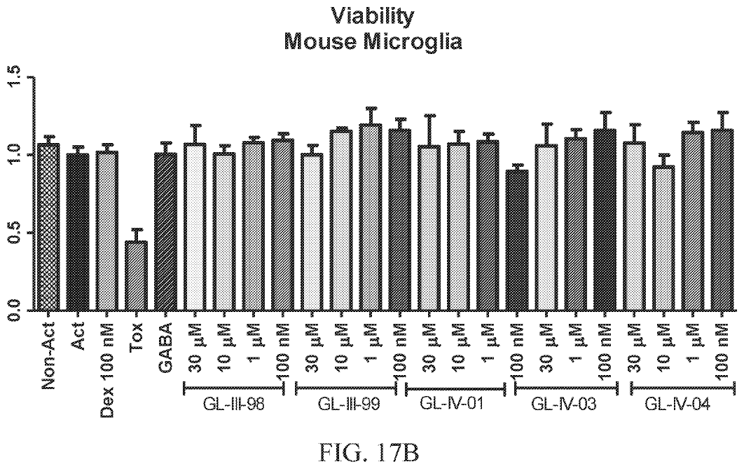

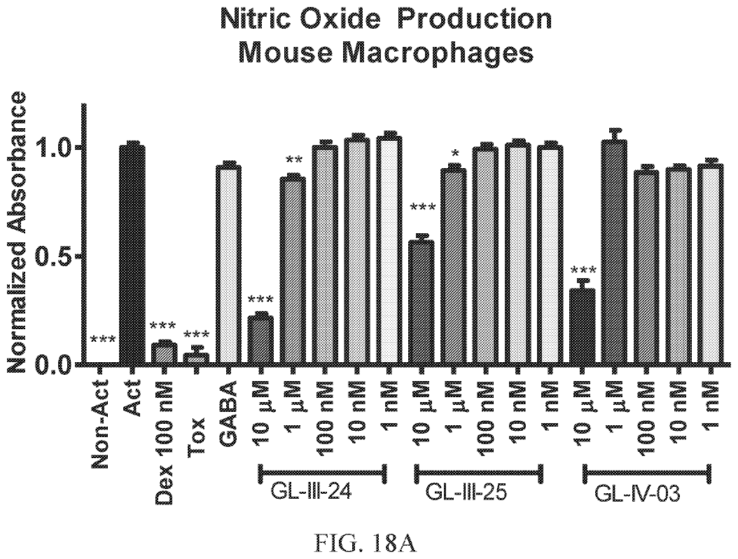

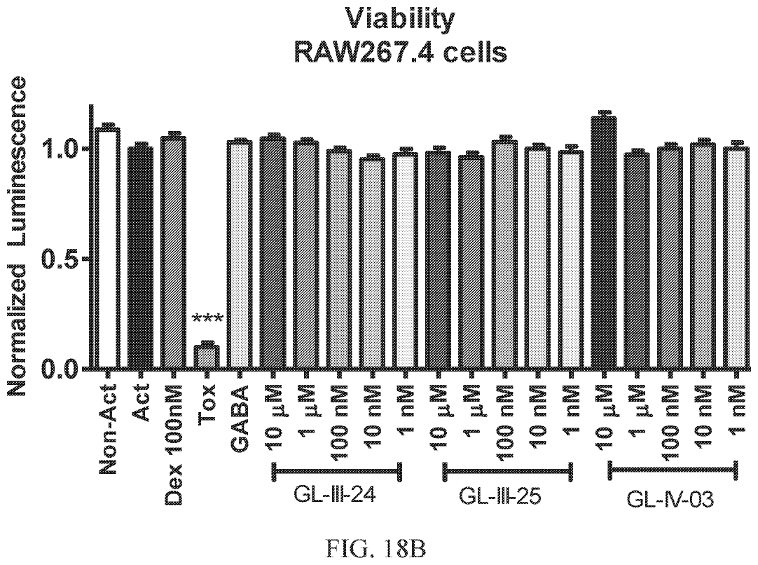

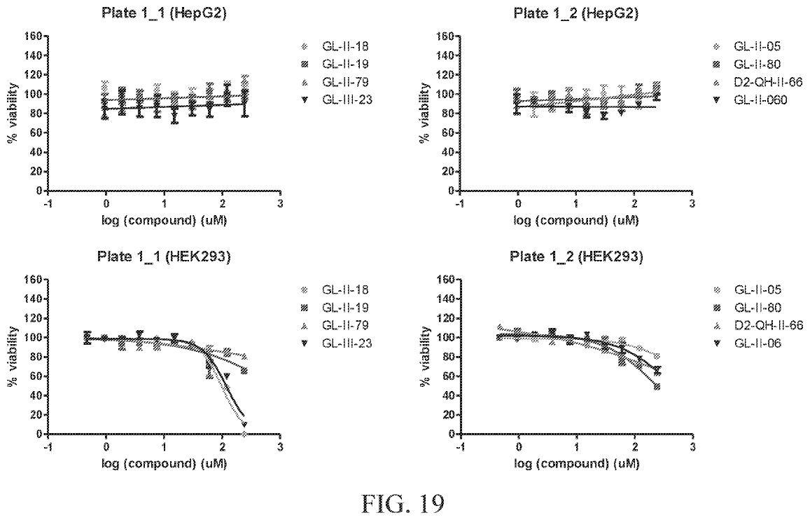

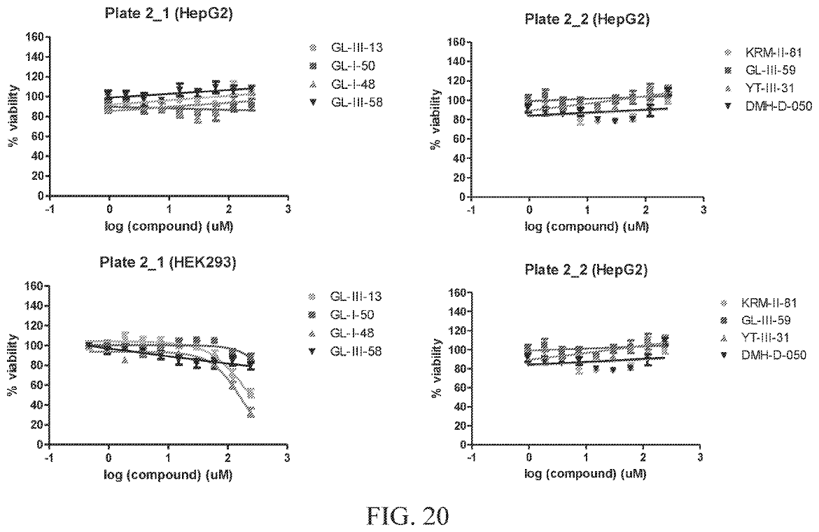

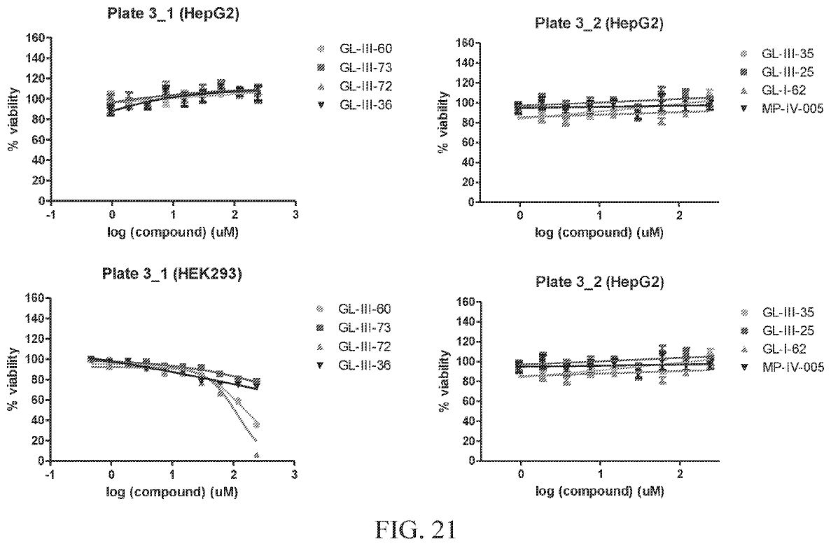

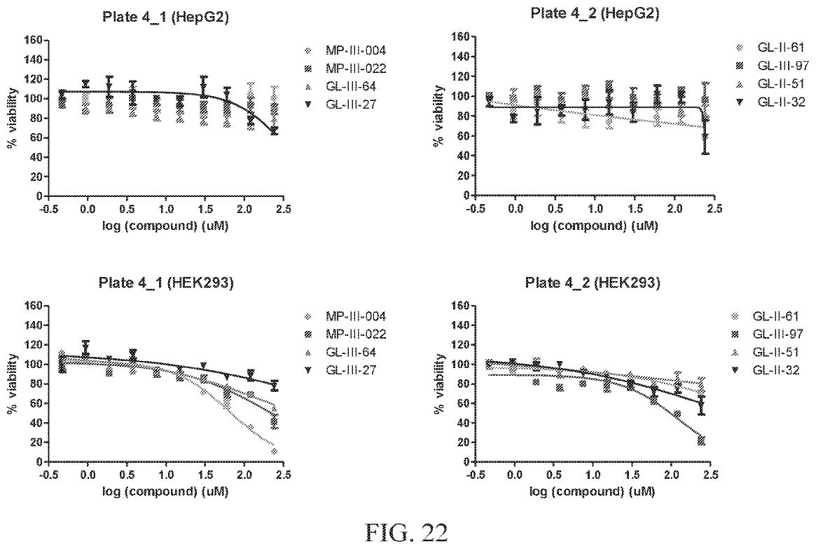

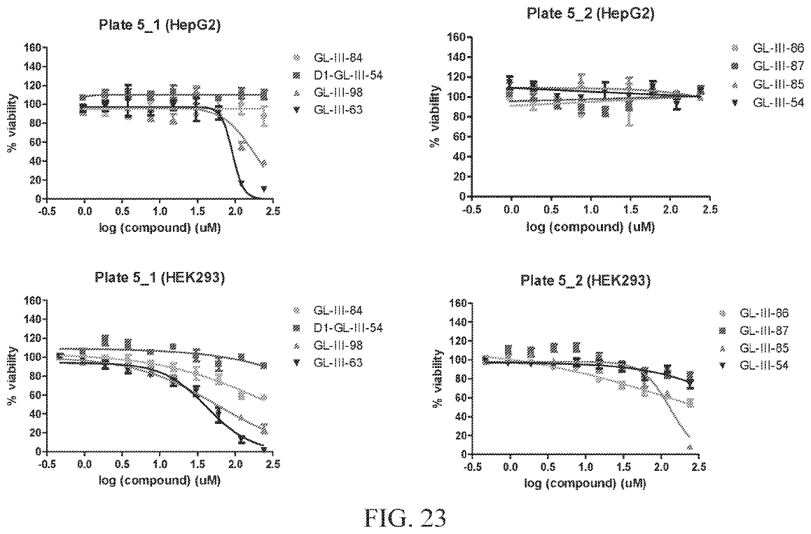

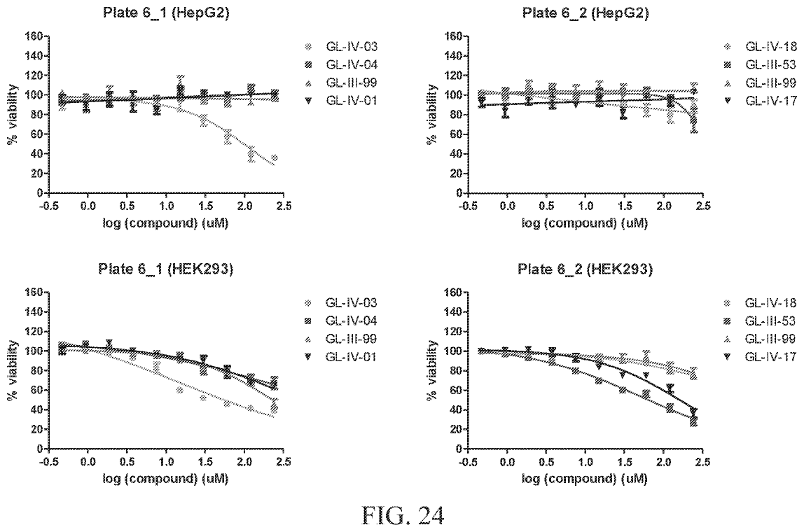

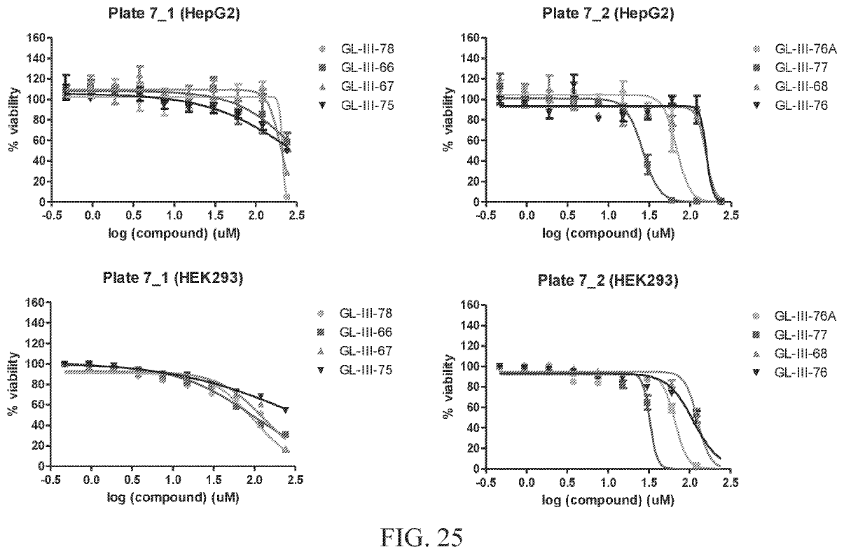

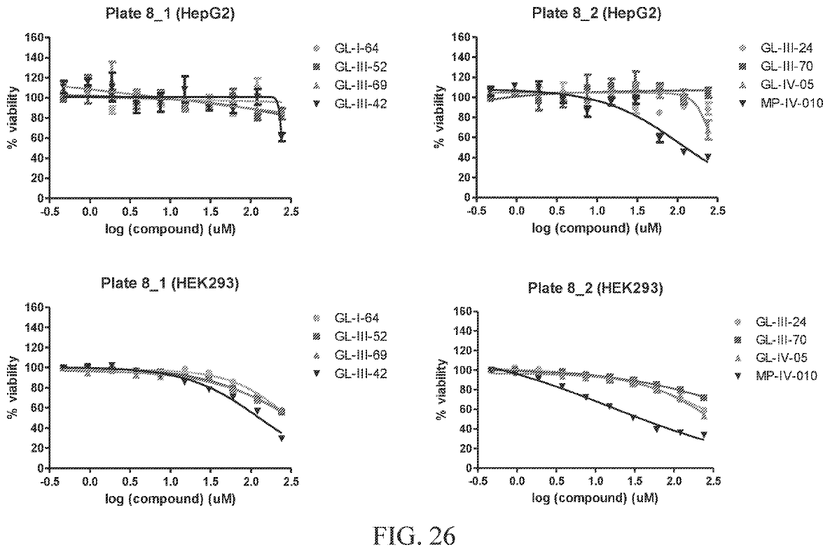

^{a}Structures are depicted in FIG. 9-14

The screen identified several compounds that reduced NO in activated microglia and macrophages. The tested compounds included achiral and chiral imidazodiazepines and benzodiazepines. In general, microglia were more sensitive to compound treatment with more pronounced reduction of NO than macrophages. The most potent compounds were analyzed at the concentrations indicated in Table 5 and FIG. 9-14. None of the compounds tested exhibited pronounced toxicity at 50 μM. Cell viability assays with HEK293 (kidney) and HepG2 (liver) cells were conducted in a concentration dependent manner. The percent viability following compound exposure at 120 μM is summarized in Table 5. A limited number of compounds, such as GL-III-63, GL-III-76A, and GL-III-77, were toxic at 120 μM. Most compounds exhibited an LD₅₀>100 μM for both cell lines.

Among the diverse set of compounds, many imidazodiazepines-[1,2,4]-oxadiazoles reduced the NO production by activated microglia. The general structure is:

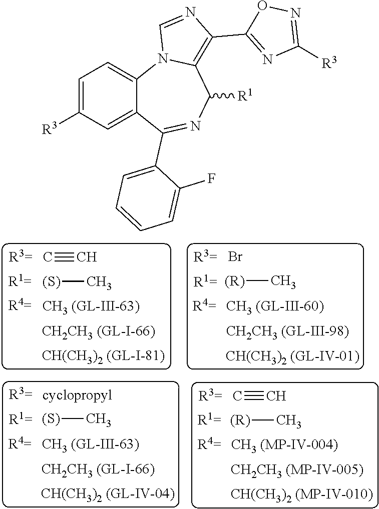

All active imidazodiazepines-[1,2,4]-oxadiazoles share the pendant 2-fluorophenyl group. Compounds with a 2-pyridine substituent, such as GL-II-33, did not reduce NO production. Second, a R¹=methyl substituent was superior to a R¹=H demonstrated by GL-III-23. Third, compounds with a (R) stereochemistry for R¹=methyl were more active than the corresponding compounds with a (S) configuration. Among the R⁴ substituents, the ethyl group was superior to the methyl or isopropyl groups for compounds bearing a bromo or cyclopropyl group in the R³ position. Among this group GL-IV-03 was the most active compound, reducing NO production by 90% for microglia and 75% in macrophages. For compounds with an acetylene substituent, MP-IV-010 was the most active compound with a R⁴ isopropyl group. For both, GL-IV-03 and MP-IV-010, no significant toxicity was observed for microglia and macrophages at 50 μM, however, at 120 μM some cytotoxicity was observed for HEK293 and to a lesser degree for HEPG2 cells.

### Example 4

### Evaluating MP-IV-010 with Regard to KOR Agonism and NO Reduction

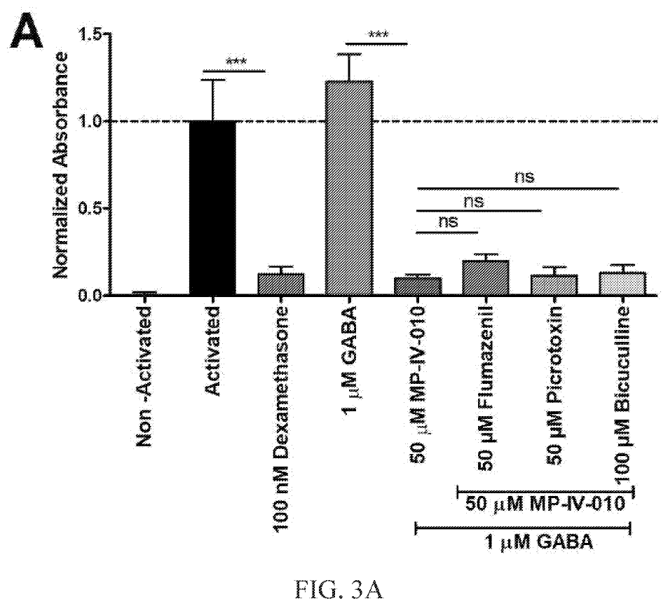

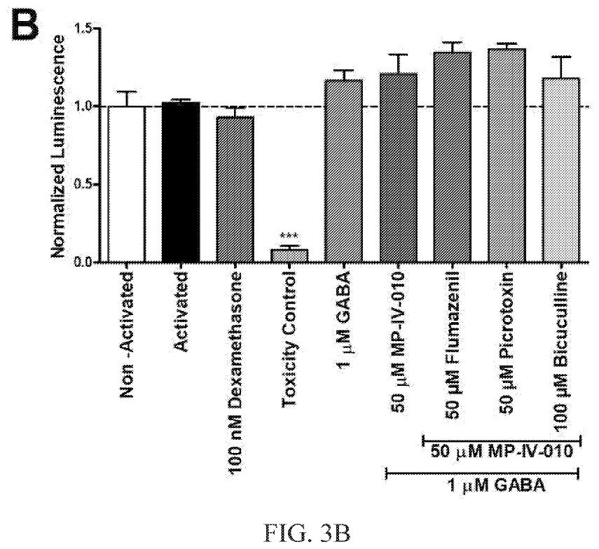

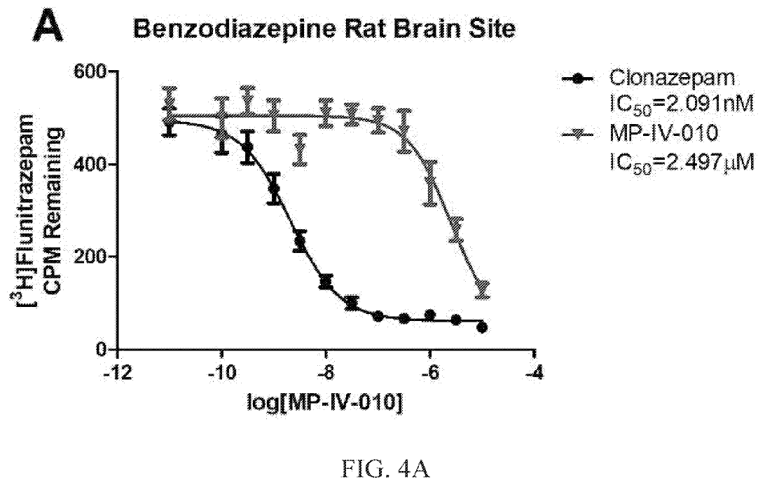

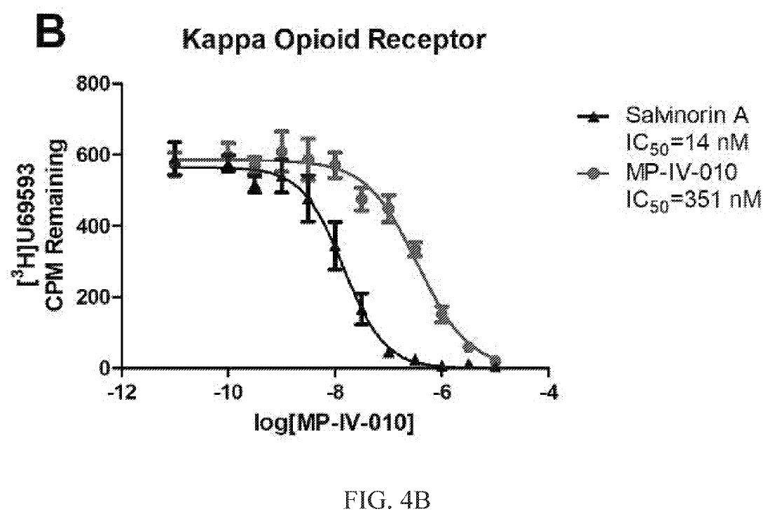

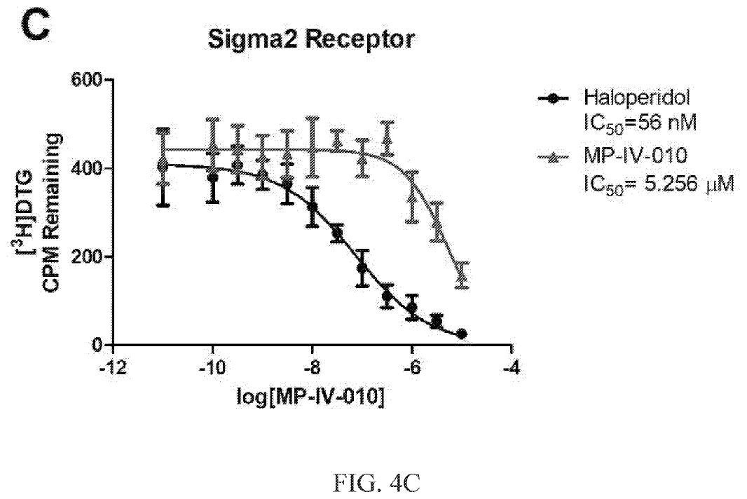

Three GABAAR antagonists were used to confirm the activity GABA_{A}R in MP-IV-010 mediated NO reduction in activated microglia. Antagonists included pierotoxin, which binds within the chloride pore of the receptor, blocking the chloride flux, and bicuculline, which competitively binds at the GABA site of the receptor and prevents endogenous activation. Flumazenil is a direct antagonist of the benzodiazepine site of GABA_{A}Rs. For the competition experiments, microglia were activated with LPS and IFNγ, and then treated with each antagonist in the presence of MP-IV-010 and GABA. The cell viability for each condition was measured with CellTiter-Glo. The results are summarized in FIG. 3A-4B.

Positive control compound dexamethasone reduced NO production of activated microglia without cell toxicity at 100 nM (FIG. 3A). GABA at 1 μM had no effect on the secreted NO levels but significant reduction was demonstrated with MP-IV-010 (50 μM). The administration of 50 μM flumazenil, 50 μM picrotoxin, or 100 μM bicuculline with 50 μM MP-IV-010 did not reverse its NO effects. No cytotoxicity was observed for any treatment conditions (FIG. 3B).

Because reduction of NO could not be reversed by GABA_{A}R antagonists, the possible binding of MP-IV-010 to other cellular receptors was evaluated in collaboration with the NIMH Psychoactive Drug Screening Program. Of the 46 receptor competition assays in the screen, MP-IV-010 demonstrated more than 50% activity for only three receptors, thus confirming its binding to the benzodiazepine site of the GABA_{A}R, but also binding to the κ-opioid receptor and the σ2 receptor (Table 6).

TABLE 6
PDSP Primary Screening Data for MP-IV-010.

| | Percent | | Percent | | Percent |
|---|---|---|---|---|---|
| Receptor | Competition | Receptor | Competition | Receptor | Competition |
| 5-HT1A | 19 | D5 | 29 | Alpha2A | 14 |
| 5-HT1B | 29 | SERT | 22 | Alpha2B | 29 |
| 5-HT1D | 6 | NET | −2 | Alpha2C | 31 |
| 5-HT1E | −7 | DAT | 14 | Beta1 | 6 |
| 5-HT2A | −1 | MOR | 33 | Beta2 | 20 |
| 5-HT2B | 0 | DOR | 5 | M1 | −11 |
| 5-HT2C | −2 | KOR | 94 | M2 | 10 |
| 5-HT3 | 13 | GABAA | −8 | M3 | 0 |
| 5-HT5A | 11 | H1 | 19 | M4 | 8 |
| 5-HT6 | 21 | H2 | 28 | M5 | 26 |
| 5-HT7 | −5 | H3 | −18 | Beta3 | 38 |
| D1 | 28 | H4 | 6 | BZP Rat | 64 |
| D2 | 25 | Calcium Channel | 15 | Brain Site | |
| D3 | 15 | Alpha1A | 2 | PBR | 28 |
| D4 | 5 | Alpha1B | 15 | Alpha1D | 9 |
| | | | | HERG binding | 29 |
| | | | | Sigma 2 | 64 |

To determine the affinity of MP-IV-010 for these receptors, concentration dependent assays were carried out as depicted in FIG. 4A-4C. The results demonstrated MP-IV-010 binding to the benzodiazepine site of GABA_{A}Rs with an affinity of 2.5 μM (IC₅₀) (FIG. 4A). The most abundant GABA_{A}R in the rat brain homogenate exhibits the α₁β_{2/3}γ₂ configuration. The affinity for the α₃β₃γ₂ GABA_{A}R was 14.3 μM (EC₅₀) determined by a membrane potential red dye assay (data not shown). Higher affinity was observed for the κ-opioid receptor with an IC₅₀ of 351 nM (FIG. 4B). The affinity for the σ2 receptor was 5.2 μM (IC₅₀) (FIG. 4C).

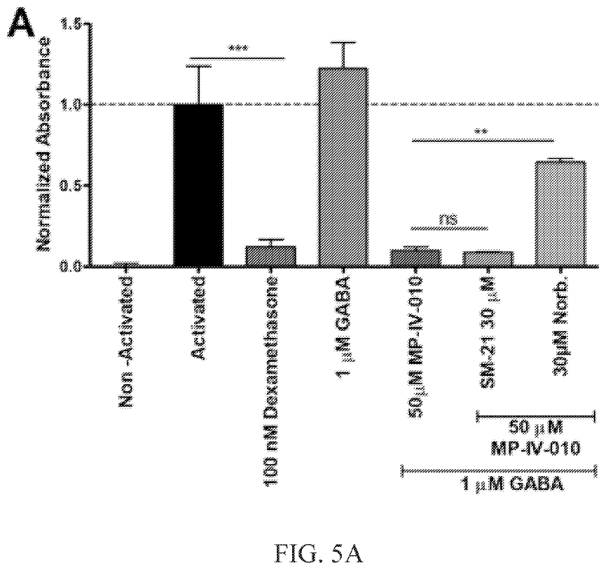

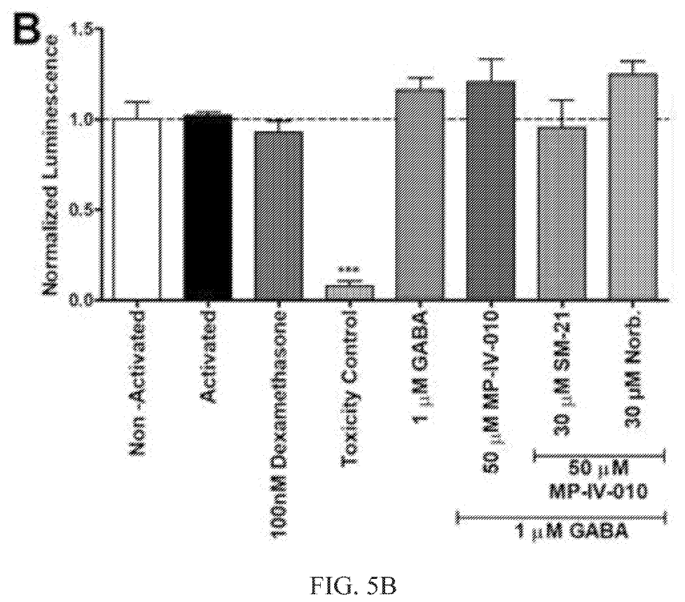

To validate the possible involvement of κ-opioid and σ2 receptors, it was investigated whether selective κ-opioid receptor antagonist norbinaltorphimine and selective σ2 receptor antagonist SM-21 could oppose reduction of NO by MP-IV-010. In the presence of SM-21 (30 μM), no reversal of NO production by MP-IV-010 (50 μM) was observed (FIG. 5A). However, norbinaltorphimine (30 μM) reversed the effects of MP-IV-010. Neither treatment reduced cell viability (FIG. 5B).

The reversal of the effects of MP-IV-010 by the κ-opioid receptor antagonist norbinaltorphimine implies that κ-opioid receptors are expressed by microglia and confirms that the activation of the x-opioid receptor regulates NO production. This data demonstrates that MP-IV-010 does not directly inhibit the enzymatic activity of iNOS, but rather modulates its expression.

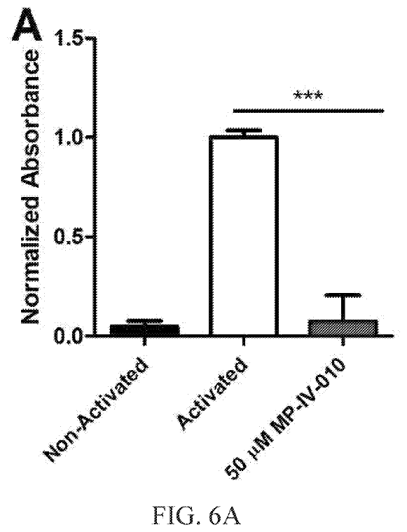

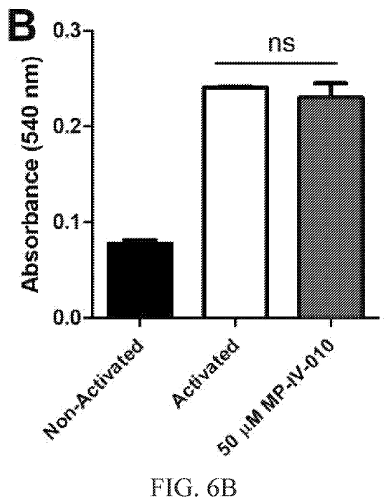

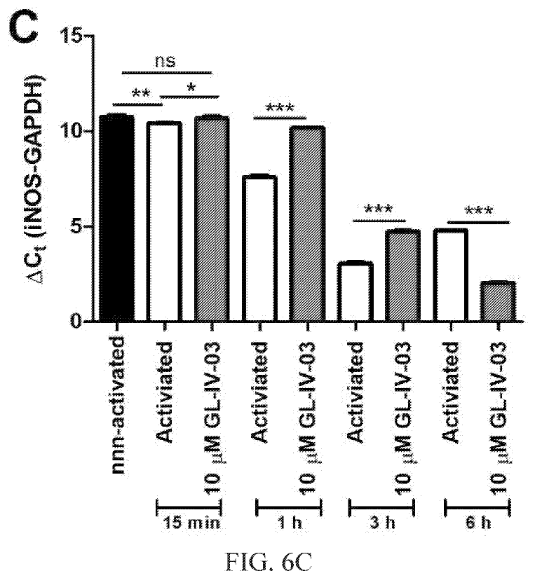

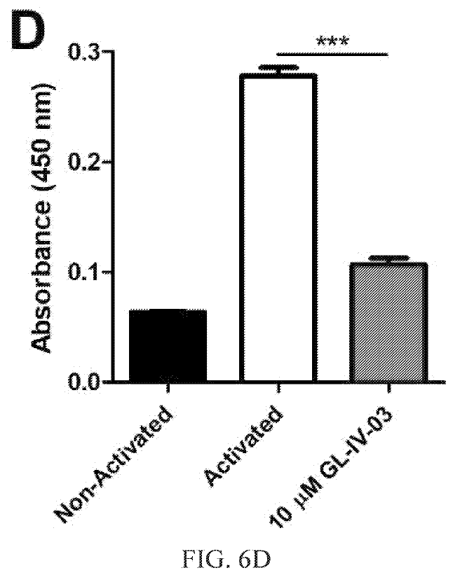

Next, the possible involvement of INOS in MP-IV-010 (and structurally similar compound GL-IV-03) mediated reduction of NO in activated microglia was investigated. INOS activity was measured in activated microglia after a 24-hour treatment with MP-IV-010. Second, proteins were harvested from microglia activated for 24 hours with LPS/INFγ, followed by a 2-hour treatment with MP-IV-010 before measuring iNOS activity. Finally, mRNA and protein levels were quantified for GL-IV-03 treated activated microglia to examine the transcriptional regulation of iNOS. Cellular protein extracts from activated microglia exhibited a significantly higher iNOS activity than extracts from non-activated microglia. MP-IV-010 (50 μM) treatment for 24 h completely inhibited the increased iNOS activity in activated microglia (FIG. 6A). To demonstrate that MP-IV-010 does not inhibit iNOS directly, cellular extracts from activated microglia were incubated with MP-IV-010 for 2 h followed by assay of iNOS activity. Here, iNOS activity did not differ from the vehicle treated cell extract (FIG. 6B) However, iNOS mRNA levels were reduced in activated microglia 15, 60, and 180 minutes after GL-IV-03 (10 μM) treatment (FIG. 6C). Interestingly, increased iNOS mRNA levels were observed after 6 h. Nevertheless, the amount of iNOS protein isolated from microglia after a 24 h treatment with GL-IV-03 was significantly lower in comparison to vehicle treated cells (FIG. 6D).

### Example 5

### Evaluation of Antinociceptive Effects of GL-IV-03 and MP-IV-010

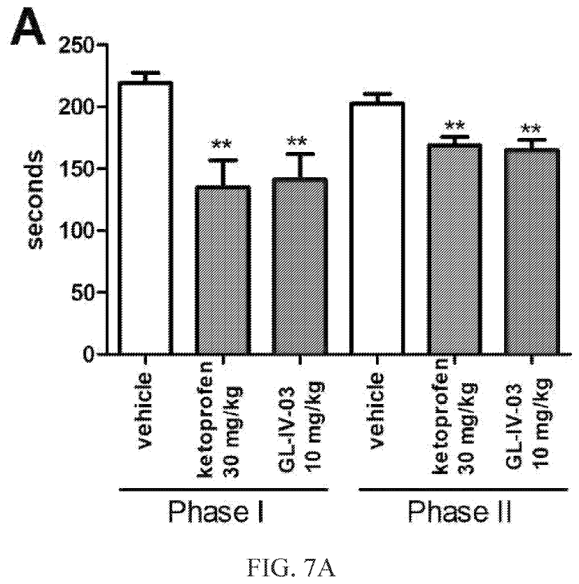

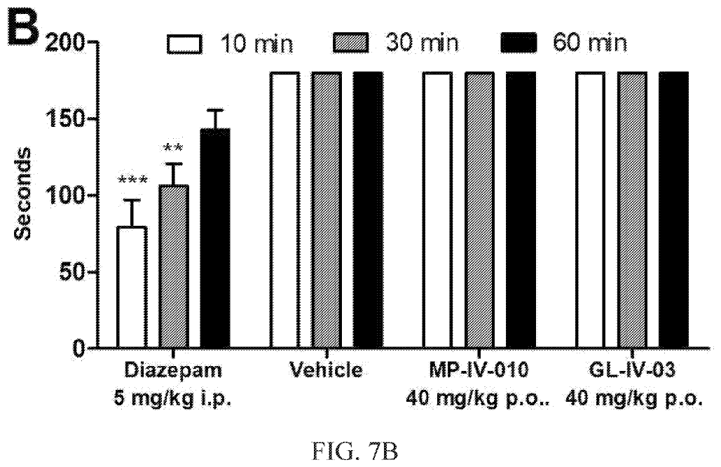

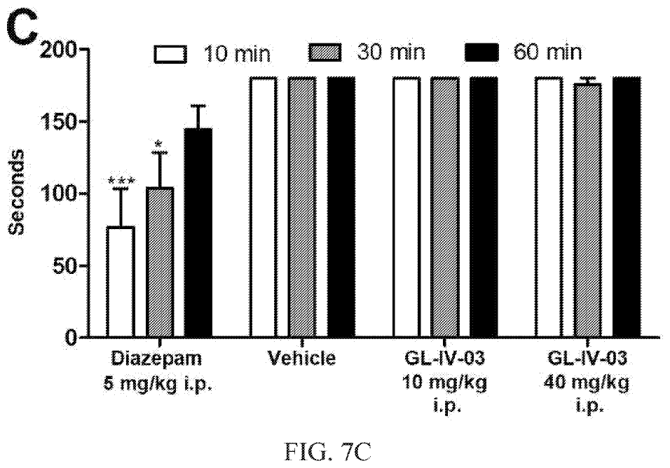

The antinociceptive effect of GL-IV-03 was investigated with the formalin test. Paw injections of formalin elicited a biphasic pain response separated in time (1-5 minutes and 20-60 minutes). Licking and biting of the injured hind paw as a demonstration of nociception was evaluated in comparison to the uninjured contralateral paw. The amount of time attending to this behavior during a five-minute interval for each treatment is presented in FIG. 7A. Sensorimotor impairment and sedation were investigated with mice that were trained to balance on a rotating rod over a period of three minutes (rotarod). The amount of time that mice remained balanced on the rod after intraperitoneal (i.p.) and oral (p.o.) treatment with GL-IV-03 and MP-IV-010 is summarized in FIG. 7B-7C. GL-IV-03 exhibited antinociceptive effects in both acute pain (phase 1) and inflammation mediated pain (phase 2) when administered orally at 10 mg/kg (FIG. 8A). The effect was similar to non-steroidal anti-inflammatory drug ketoprofen at 30 mg/kg. Sedation and inhibition of sensorimotor coordination can influence licking and biting pain behaviors, prompting rotarod studies for GL-IV-03 and MP-IV-010. Both compounds administered p.o. at 4-fold the effective dose in the formalin test did not cause any sensorimotor deficits as measured on the rotarod (FIG. 7B). Furthermore, intraperitoneal injections of GL-IV-03 at 10 and 40 mg/kg did not influence the ability of mice to balance on a rotating rod for three minutes (FIG. 7C). In contrast to diazepam, a benzodiazepine with high affinity for the α₁β_{2/3}γ₂ and other GABA_{A}R subtypes, was sedating at 5 mg/kg.

### Detailed Protocols for Examples 3-5

Animals. 5 to 10-week-old male Swiss Webster mice (Charles River Laboratory, WIL, MA) were housed under specific pathogen-free conditions, under standard conditions of humidity, temperature, and a controlled 12 h light and dark cycle and ad libidum access to food and water. All animal experiments were conducted in compliance with the University of Wisconsin-Milwaukee Institutional Animal Care and Use Committee (IACUC).

Cell culture. A) HMC3 Microglia (CRL-3304) were maintained in treated cell culture flasks (Nunc, 156472) with EMEM (Corning, 10010CV) supplemented with 10% heat-inactivated FBS (Corning, 35011CV) and 100 U/mL penicillin and 100 μg/mL streptomycin (Hyclone, SV30010). Cultures were maintained at 37° C. and 5% CO₂. When cells reached a density of 7×10⁴ cells/mL, cultures were passaged with 0.25% Trypsin (Corning, 25-053-CI) and resuspended to a density no lower than 10,000 cells/mL. B) Human and mouse microglia cell lines were a gift from Dr. David Alvarez-Carbonell at Case Western Reserve University (Cleveland, OH). Microglia were cultured in cell culture treated flasks (Nunc, 156472) with DME/F12 media (Hyclone, SH30023.01) supplemented with 10% heat-inactivated FBS (Corning, 35011CV), 100 U/mL penicillin/100 μg/mL streptomycin (Hyclone, SV30010), and Normocin (Invivogen, ant-nr). Cultures were maintained at 37° C. and 5% CO₂. Cultures were passaged with 0.25% Trypsin (Corning, 25-053-CI) when cells reached a density of approximately 500,000 cells/mL and resuspended at densities no lower than 50,000 cells/mL; C) RAW264.7 Murine Macrophages (ATCC, TIB-71) were cultured in non-treated flasks (CellStar, 658195) with DMEM/High Glucose media (Hyclone, SH30243.01) supplemented with with 10% heat-inactivated FBS (Corning, 35011CV), 100 U/mL penicillin/100 μg/mL streptomycin (Hyclone, SV30010), and Normocin (Invivogen, ant-nr). Cultures were maintained at 37° C. and 5% CO₂. Cells were passaged when they reached a density of approximately 3×10⁶ cells/mL using cell scrapers (VWR, 10062-906) to lift cells from flask surface and resuspended at densities of approximately 500,000 cells/mL.

Automated patch clamp. After isolation, microglia were centrifuged at 380 g for 2 min and gently suspended in extracellular solution (in mM: NaCl 140, KCl 5, CaCl₂, MgCl₂ 1, glucose 5, HEPES 10, pH 7.4 with NaOH) at a concentration of 5×10⁶ cells/ml. Automated patch clamp assays were conducted with the lonFlux16 instrument as described previously (Yuan et al., J. Pharmacol. Toxicol. Methods 82, 109-114; Chen et al., Assay Drug Dev. Technol. 10, 325-335). Briefly, the lonFlux16 plates consist of 8 patterns, each containing 8 concentration wells: 1 inlet for cell supply, 1 outlet for waste collection, and 2 traps that contain combs that can patch 20 cells per experiment (for a total of 40 cells per pattern). The inlet wells contain intracellular solution (in mM: CsCl 140, CaCl₂ 1, MgCl₂ 1, EGTA 11, and HEPES 10, pH 7.2 with CsOH). The cells were suspended in extracellular solution. The 8 concentration wells contained GABA diluted in extracellular solution. Cells are captured in the traps through a pulse of suction, then whole cell recording access is obtained through a second strong pulse of suction that breaks the membrane. GABA application was achieved by applying pressure onto the appropriate well. Cells are voltage clamped at a holding potential of −80 mV. Current data (n=16) were normalized in response to maximum current measured with 1 mM GABA and depicted as mean and SEM. EC₅₀ values were calculated by non-linear regression.

GABA_{A}R subunit RT-qPCR. mRNA was collected from 5×10⁶ microglia cells or 23.5 mg of brain tissue from female CFW mice (Charles River, 024CFW) using QIAshredder (Qiagen, 79654) and RNeasy Mini Kit (Qiagen, 74104). Human whole brain RNA was purchased (BioChain, R¹⁴³⁴⁰³⁵-50). 50 ng/reaction of microglial mRNA and 10 ng/reactopm of whole brain mRNA was analyzed using the QuantiFast SYBR Green RT-PCR kit (Qiagen, 204154). Data were acquired using an Eppendorf Mastercycler Pro and analyzed by the Delta Cₜ method. Each measurement was carried out in triplicate. All PCR products were separated with agarose gel electrophoresis (2%) to verify a single band for each primer pair.

Nitric oxide production (Griess assay) and viability assay (CellTiter-Glo). 80 μL of mouse microglia culture (1×10⁶ cells/mL) was plated into sterile 384 well plates representing non-activated wells. The remaining culture was activated with 50 ng/ml LPS (Invivogen, NC9836) and 150 U/mL IFNγ (R&D Systems, 485MI100) and distributed into the 384 well plates. 0.1 μL of 800 μM GABA diluted in MilliQ water (final concentration of 1 μM) and appropriate concentrations of compounds of interest diluted in DMSO were added via a TECAN EVO liquid handling system equipped with a 100 nL pin tool (V&P Scientific). Assay plates were incubated for approximately 24 hours (unless otherwise noted) at 37° C., centrifuged, and 40 μL of supernatant transferred to a new plate for analysis using the Griess Assay (Promega, Madison, WI). Absorbance at 530 nm was measured using a TECAN Infinite M1000 plate reader. The remaining 40 μL containing cells was analyzed for toxicity by the CellTiter-Glo Assay (Promega, Madison, WI). Luminescence was read using a TECAN Infinite M1000 plate reader.

Viability assay. Human liver hepatocellular carcinoma (HEPG2) and human embryonic kidney 293T (HEK293T) cell lines were purchased (ATCC) and cultured in 75 cm² flasks (CellStar). Cells were grown in DMEM/High Glucose (Hyclone, #SH3024301) media to which non-essential amino acids (Hyclone, #SH30238.01), 10 mM HEPES (Hyclone, #SH302237.01), 5×10⁶ units of penicillin and streptomycin (Hyclone, #SV30010), and 10% of heat inactivated fetal bovine serum (Gibco, #10082147) were added. Cells were harvested using 0.05% Trypsin (Hyclone, #SH3023601), washed with PBS, and dispensed into sterile white, optical bottom 384-well plates (NUNC, #142762). After three hours, small molecule DMSO solutions were transferred with a Tecan Freedom EVO liquid handling system equipped with a 100 nL pin tool (V&P Scientific). The controls were 3-dibutylamino-1-(4-hexyl-phenyl)-propan-1-one (25 mM in DMSO, positive control) and DMSO (negative control). The cells were incubated for 48 hours followed by the addition of CellTiter-Glo™, a luminescence-based cell viability assay (Promega, Madison, WI). All luminescence readings were performed on a Tecan Infinite M1000 plate reader. The assay was carried out in quadruplet with two independent runs. The data were analyzed by nonlinear regression.

Psychoactive Drug Screening Program. Detailed protocols for the primary and secondary radioligand binding assays can be found in the National Institute of Mental Health's Psychoactive Drug Screening Program (NIMH PDSP) Assay Protocol Book. Briefly, primary and secondary radioligand binding assays are carried out in a final of volume of 125 μL per well in appropriate binding buffer. The radioactive ligand concentration is close to the Kd. Total binding and nonspecific binding are determined in the absence and presence of 10 μM of appropriate reference compound, respectively. In brief, plates are usually incubated at room temperature and in the dark for 90 min. Reactions are stopped by vacuum filtration onto 0.3% polyethyleneimine (PEI) soaked 96-well filter mats using a 96-well Filtermate harvester, followed by three washes with cold PBS buffer. Scintillation cocktail is then melted onto the microwave-dried filters on a hot plate and radioactivity counted in a Microbeta counter. The data (n=6) were analyzed by nonlinear regression.

iNOS activity assay. Mouse microglia were activated with 50 ng/mL LPS (Invivogen, NC9836) and 150 U/mL IFNγ (R&D Systems, 485MI100), and treated with compounds of interest in 6 well plates for 2 or 24 hours. Final DMSO concentrations did not exceed 0.1%. Protein was collected, quantified, and analyzed with the Nitric Oxide Synthase Activity kit (Abcam, ab211083) with minor alterations to the established protocol. Briefly, protein was incubated with cofactors and substrates in 96 well plates for 2.5 hours at 37° C. and 5% CO₂. Enhancer was added to each well and incubated for 10 min before Griess reagents were added and incubated. Absorbance at 540 nm was measured using a TECAN Infinite M1000 plate reader. Significance (n=3) was analyzed by ANOVA.

iNOS RT-qPCR. 1×10⁶ mouse microglia cells were plated in 6 well plates (VWR, 10062-892) in 1-2 mL of media, activated with 50 ng/ml LPS (Invivogen, NC9836) and 150 U/mL IFNγ (R&D Systems, 485MI100), and treated with compounds of interest. Final DMSO concentration did not exceed 0.1%. Plates were incubated for stated times at 37° C. and 5% CO₂. mRNA was collected using QIAshredder (Qiagen, 79654) and RNeasy Mini Kit (Qiagen, 74104). mRNA was analyzed using the QuantiFast SYBR Green RT-PCR kit (Qiagen, 204154) along with iNOS primers (F. 5′-ACA TCA GGT CGG CCA TCA CT-3′, R: 5′-CGT ACC GGA TGA GCT GTG AAT-3′). Expected product size was 87 bp. Data were acquired using an Eppendorf Mastercycler Pro and analyzed by the Delta Cₜ method. Each measurement was carried out in triplicate and significance analyzed by ANOVA. All PCR products were separated with agarose gel electrophoresis (2%) to verify a single band for each primer pair.

iNOS ELISA. Mouse microglia (2 mL of 500,000 cells/mL cell solution) were activated with 50 ng/mL LPS (Invivogen, NC9836) and 150 U/mL IFNγ (R&D Systems, 485M1100), and treated with compounds of interest in 6 well plates (VWR, 10062-892) for 24 hours. Final DMSO concentrations did not exceed 0.1%. Cells were lysed with RIPA buffer and protein concentration quantified with the Pierce Rapid Gold BCA Protein Assay Kit (Thermo Scientific, A53227). Samples at 50 μg/mL were analyzed with the Mouse iNOS ELISA kit (ABCAM, ab253219). Absorbance (λ: 450 nm) was measured using a TECAN Infinite M1000 plate reader. Each measurement was carried with an n=3 and significance analyzed by ANOVA.

Formalin test. GL-IV-03 and ketoprofen was formulated in 2.5% polyethylene glycol and 2% hydroxypropyl methylcellulose. Each treatment was administered over a period of four days by oral gavage at 10 mL/kg once a day. Twenty minutes after the administration, 20 μL of 2% formalin in 0.9% saline solution was administered to the right hind paw via intraplantar injection while restrained. Immediately after the hind paw injection, mice were removed and placed into the observation chamber for 5 minutes to measure Phase I behavior, representing acute peripheral pain mediated by direct activation of nociceptors. The animal was observed in 5 seconds intervals and occurrence of licking and biting the injected paw was recorded to determine to a pain score of 3 as defined in the Dubuisson and Dennis Model. Mice were placed back in their home cage for a 15-minute quiescent period and reanalyzed for 5 minutes to measure Phase II behavior, representative of an inflammatory and/or neurogenic response. The group size was six and unpaired t-test was used to determine significance in comparison to the vehicle group

Rotarod test: Female Swiss Webster mice were trained to maintain balance at a constant speed of 15 rpm on the rotarod apparatus (Omnitech Electronics Inc., Nova Scotia, Canada) until mice could perform for three minutes at three consecutive time points. Separate groups of mice (n=10) received intraperitoneal (i.p.) injections of compounds (10% DMSO, 40% propylene glycol and 50% PBS) at 5 mL/kg or orally administrated compounds (2.5% polyethylene glycol and 2% hydroxypropyl methylcellulose) at 10 mL/kg. 10, 30 and 60 min after each injection, mice were placed on the rotarod for 3 min. If a mouse fell before the three minutes were completed, it was placed again on the rod. If a mouse fell for the second time, the time of the fall was recorded. Significance was calculated by 2-way ANOVA.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses. compositions. formulations, or methods of use of the invention, may be made without departing from the spirit and scope thereof.

What is claimed is:

1. A method of treating an inflammatory disease or disorder in a subject comprising administering the subject, a therapeutically effective amount of a compound of formula (I), or a pharmaceutically acceptable salt thereof, 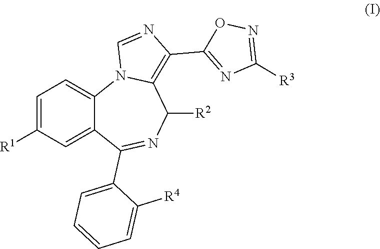
wherein:
R¹ is C₃₋₄cycloalkyl, halogen, ethynyl, —OC₁₋₂alkyl, or phenyl;
R² is methyl;
R³ is C₁₋₄alkyl or C₃₋₄cycloalkyl; and
R⁴ is hydrogen or halogen.

2. A method of inhibiting inducible nitric oxide synthase (iNOS) in a subject comprising administering to the subject, an amount of a compound of formula (1), or a pharmaceutically acceptable salt thereof, that is effective to inhibit iNOS in the subject, 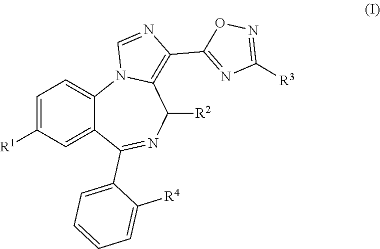
wherein:
R¹ is C₃₋₄cycloalkyl, halogen, ethynyl, —OC₁₋₂alkyl, or phenyl;
R² is methyl;
R³ is C₁₋₄alkyl or C₃₋₄cycloalkyl; and
R⁴ is hydrogen or halogen.

3. A method of activating a k-opioid receptor (KOR) in a subject comprising administering to the subject, an amount of a compound of formula (I), or a pharmaceutically acceptable salt thereof, that is effective to activate KOR in the subject, 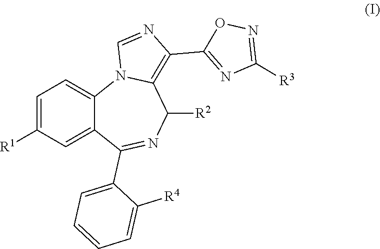
wherein:
R¹ is C₃₋₄cycloalkyl, halogen, ethynyl, —OC₁₋₂alkyl, or phenyl;
R² is methyl;
R³ is C₁₋₄alkyl or C₃₋₄cycloalkyl; and
R⁴ is hydrogen or halogen.

4. The method of any of claims 1-3, wherein R¹ is cyclopropyl.

5. The method of any of claims 1-3, wherein R¹ is ethynyl.

6. The method of any of claims 1-3, wherein R¹ is bromo.

7. The method of any of claims 1-6, wherein R³ is C₁₋₄alkyl.

8. The method of any of claims 1-7, wherein R³ is methyl.

9. The method of any of claims 1-7, wherein R³ is ethyl.

10. The method of any of claims 1-7, wherein R³ is isopropyl.

11. The method of any of claims 1-10, wherein R⁴ is fluoro.

12. The method of any of claims 1-11, wherein formula (I) is formula (Ia)

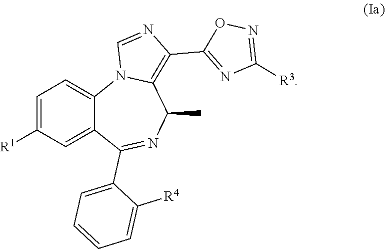

13. The method of any of claims 1-11, wherein formula (D) is formula (Ib)

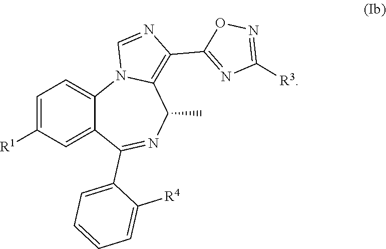

14. The method of any of claims 1-13, wherein the compound of formula (I) is selected from the group consisting of:

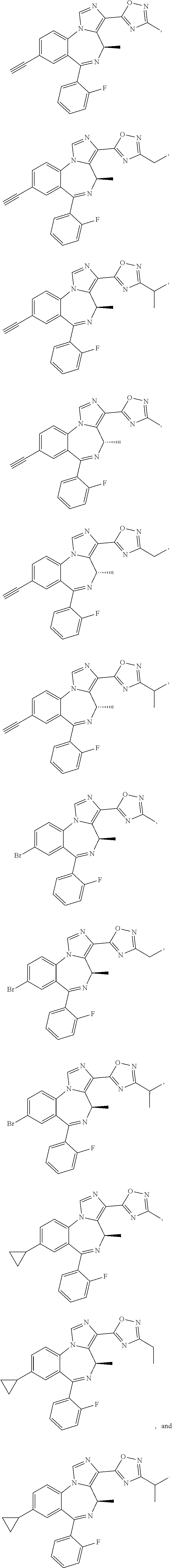

15. The method of any of claims 1-14, wherein the subject suffers from an inflammatory disease or disorder.

16. The method of claim 15, wherein the inflammatory disease or disorder is an acute inflammatory disease or disorder.

17. The method of claim 15, wherein the inflammatory disease or disorder is a chronic inflammatory disease or disorder.

18. The method of claim 15, wherein the inflammatory disease or disorder is a respiratory disease or condition.

19. The method of claim 18, wherein the respiratory disease or condition is asthma, chronic obstructive pulmonary disease, bronchitis, emphysema, inflammatory diseases of the upper respiratory tract such as allergic rhinitis and allergic sinusitis, acute lung injury, acute respiratory distress syndrome (ARDS), lung infection, interstitial lung disease, or constrictive bronchiolitis.

20. The method of claim 15, wherein the inflammatory disease or disorder is a digestive tract disease or condition.

21. The method of claim 20, wherein the disease or disorder is an inflammatory bowel disease, Crohn's disease, ulcerative colitis, esophagitis, irritable bowel syndrome, celiac disease, gastritis, pancreatitis, proctitis, hepatitis, diverticulitis, or tropical sprue.

22. The method of claim 15, wherein the inflammatory disease or disorder is a dermatologic disease or condition.

23. The method of claim 22, wherein the disease or disorder is dermatitis, atopic dermatitis, rash, pruritis, eczema, acne, dandruff, cellulitis, psoriasis, rosacea, hives, shingles, lupus erythematosus, lichen planus, dermatitides, vasculitis, or bullous diseases.

24. The method of claim 15, wherein the inflammatory disease or disorder is septic shock, sepsis, systemic inflammatory response syndrome (SIRS), hemorrhagic shock, shock states induced by cytokine therapy (interleukin-2, tumor necrosis factor, immune checkpoint inhibition), organ transplantation and transplant rejection, bead trauma, or inflammatory eye conditions such as uveitis, glaucoma and conjunctivitis.

25. The method of claim 15, wherein the inflammatory disease or disorder is an arthritic disorder such as rheumatoid arthritis, ankylosing spondylitis, osteoarthritis and gouty arthritis, a heart disorder such as cardiomyopathy and myocarditis, atherosclerosis, neurogenic inflammation, diabetes, glomerulonephritis; a urological disorder such as overactive bladder and cystitis Parkinson's disease, Huntington's induced dementias, amyotrophic lateral sclerosis (ALS), multiple sclerosis; necrotizing vasculitides such as polyarteritis nodosa, serum sickness, Wegener's granulomatosis, Kawasaki's syndrome; headaches such as migraine, chronic tension headaches, cluster and vascular headaches, or myocardial and cerebral ischemia/reperfusion injury.

26. The method of any of claims 1-25, wherein the compound, or pharmaceutically acceptable salt thereof is administered orally, topically, parenterally, or by inhalation.

27. The method of any of claims 1-26, wherein the subject is a human or non-human animal.

28. A compound selected from the group consisting of: 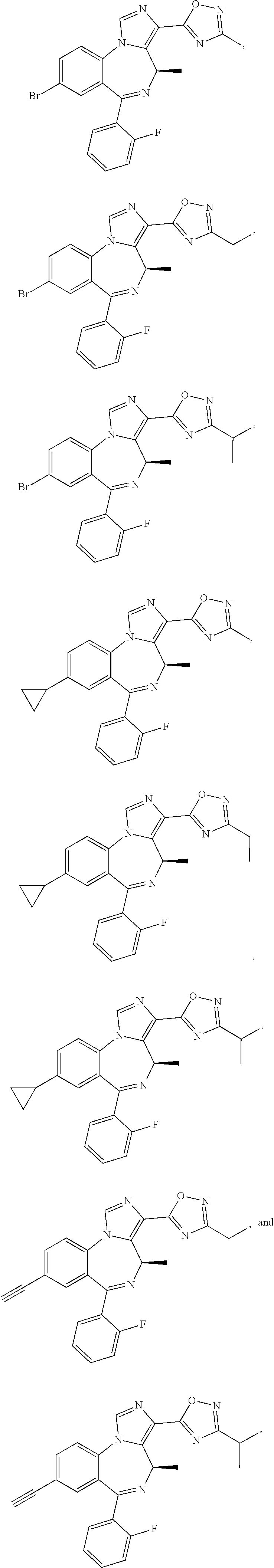
or a pharmaceutically acceptable salt thereof.

29. A pharmaceutical composition comprising a compound of claim 28, or a pharmaceutically acceptable salt thereof, and a pharmaceutically acceptable carrier.
